SEQUENCE LISTING (13 sequences)

SEQ ID NO: 1 - DNA, 8157 bp, Artificial Sequence
  pCMV/R Chikungunya (strain 37997)
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattggactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagacaccatggagttcatcccgacgcaaactttctataacagaaggtaccaaccccgaccctgggccccacgccctacaattcaagtaattagacctagaccacgtccacagaggcaggctgggcaactcgcccagctgatctccgcagtcaacaaattgaccatgcgcgcggtacctcaacagaagcctcgcagaaatcggaaaaacaagaagcaaaggcagaagaagcaggcgccgcaaaacgacccaaagcaaaagaagcaaccaccacaaaagaagccggctcaaaagaagaagaaaccaggccgtagggagagaatgtgcatgaaaattgaaaatgattgcatcttcgaagtcaagcatgaaggcaaagtgatgggctacgcatgcctggtgggggataaagtaatgaaaccagcacatgtgaagggaactatcgacaatgccgatctggctaaactggcctttaagcggtcgtctaaatacgatcttgaatgtgcacagataccggtgcacatgaagtctgatgcctcgaagtttacccacgagaaacccgaggggtactataactggcatcacggagcagtgcagtattcaggaggccggttcactatcccgacgggtgcaggcaagccgggagacagcggcagaccgatcttcgacaacaaaggacgggtggtggccatcgtcctaggaggggccaacgaaggtgcccgcacggccctctccgtggtgacgtggaacaaagacatcgtcacaaaaattacccctgagggagccgaagagtggagcctcgccctcccggtcttgtgcctgttggcaaacactacattcccctgctctcagccgccttgcacaccctgctgctacgaaaaggaaccggaaagcaccttgcgcatgcttgaggacaacgtgatgagacccggatactaccagctactaaaagcatcgctgacttgctctccccaccgccaaagacgcagtactaaggacaattttaatgtctataaagccacaagaccatatctagctcattgtcctgactgcggagaagggcattcgtgccacagccctatcgcattggagcgcatcagaaatgaagcaacggacggaacgctgaaaatccaggtctctttgcagatcgggataaagacagatgacagccacgattggaccaagctgcgctatatggatagccatacgccagcggacgcggagcgagccggattgcttgtaaggacttcagcaccgtgcacgatcaccgggaccatgggacactttattctcgcccgatgcccgaaaggagagacgctgacagtgggatttacggacagcagaaagatcagccacacatgcacacacccgttccatcatgaaccacctgtgataggtagggagaggttccactctcgaccacaacatggtaaagagttaccttgcagcacgtacgtgcagagcaccgctgccactgctgaggagatagaggtgcatatgcccccagatactcctgaccgcacgctgatgacgcagcagtctggcaacgtgaagatcacagttaatgggcagacggtgcggtacaagtgcaactgcggtggctcaaacgagggactgacaaccacagacaaagtgatcaataactgcaaaattgatcagtgccatgctgcagtcactaatcacaagaattggcaatacaactcccctttagtcccgcgcaacgctgaactcggggaccgtaaaggaaagatccacatcccattcccattggcaaacgtgacttgcagagtgccaaaagcaagaaaccctacagtaacttacggaaaaaaccaagtcaccatgctgctgtatcctgaccatccgacactcttgtcttaccgtaacatgggacaggaaccaaattaccacgaggagtgggtgacacacaagaaggaggttaccttgaccgtgcctactgagggtctggaggtcacttggggcaacaacgaaccatacaagtactggccgcagatgtctacgaacggtactgctcatggtcacccacatgagataatcttgtactattatgagctgtaccccactatgactgtagtcattgtgtcggtggcctcgttcgtgcttctgtcgatggtgggcacagcagtgggaatgtgtgtgtgcgcacggcgcagatgcattacaccatatgaattaacaccaggagccactgttcccttcctgctcagcctgctatgctgcgtcagaacgaccaaggcggccacatattacgaggctgcggcatatctatggaacgaacagcagcccctgttctggttgcaggctcttatcccgctggccgccttgatcgtcctgtgcaactgtctgaaactcttgccatgctgctgtaagaccctggcttttttagccgtaatgagcatcggtgcccacactgtgagcgcgtacgaacacgtaacagtgatcccgaacacggtgggagtaccgtataagactcttgtcaacagaccgggttacagccccatggtgttggagatggagctacaatcagtcaccttggaaccaacactgtcacttgactacatcacgtgcgagtacaaaactgtcatcccctccccgtacgtgaagtgctgtggtacagcagagtgcaaggacaagagcctaccagactacagctgcaaggtctttactggagtctacccatttatgtggggcggcgcctactgcttttgcgacgccgaaaatacgcaattgagcgaggcacatgtagagaaatctgaatcttgcaaaacagagtttgcatcggcctacagagcccacaccgcatcggcgtcggcgaagctccgcgtcctttaccaaggaaacaacattaccgtagctgcctacgctaacggtgaccatgccgtcacagtaaaggacgccaagtttgtcgtgggcccaatgtcctccgcctggacaccttttgacaacaaaatcgtggtgtacaaaggcgacgtctacaacatggactacccaccttttggcgcaggaagaccaggacaatttggtgacattcaaagtcgtacaccggaaagtaaagacgtttatgccaacactcagttggtactacagaggccagcagcaggcacggtacatgtaccatactctcaggcaccatctggcttcaagtattggctgaaggaacgaggagcatcgctacagcacacggcaccgttcggttgccagattgcgacaaacccggtaagagctgtaaattgcgctgtggggaacataccaatttccatcgacataccggatgcggcctttactagggttgtcgatgcaccctctgtaacggacatgtcatgcgaagtaccagcctgcactcactcctccgactttgggggcgtcgccatcatcaaatacacagctagcaagaaaggtaaatgtgcagtacattcgatgaccaacgccgttaccattcgagaagccgacgtagaagtagaggggaactcccagctgcaaatatccttctcaacagccctggcaagcgccgagtttcgcgtgcaagtgtgctccacacaagtacactgcgcagccgcatgccaccctccaaaggaccacatagtcaattacccagcatcacacaccacccttggggtccaggatatatccacaacggcaatgtcttgggtgcagaagattacgggaggagtaggattaattgttgctgttgctgccttaattttaattgtggtgctatgcgtgtcgtttagcaggcactaatgaggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgccttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattcatgacattaacctataaaaataggcgtatcacgaggccctttcgtc

SEQ ID NO: 2 - DNA, 3744 bp, Artificial Sequence
  Chikungunya (strain 37997) structural proteins
atggagttcatcccgacgcaaactttctataacagaaggtaccaaccccgaccctgggccccacgccctacaattcaagtaattagacctagaccacgtccacagaggcaggctgggcaactcgcccagctgatctccgcagtcaacaaattgaccatgcgcgcggtacctcaacagaagcctcgcagaaatcggaaaaacaagaagcaaaggcagaagaagcaggcgccgcaaaacgacccaaagcaaaagaagcaaccaccacaaaagaagccggctcaaaagaagaagaaaccaggccgtagggagagaatgtgcatgaaaattgaaaatgattgcatcttcgaagtcaagcatgaaggcaaagtgatgggctacgcatgcctggtgggggataaagtaatgaaaccagcacatgtgaagggaactatcgacaatgccgatctggctaaactggcctttaagcggtcgtctaaatacgatcttgaatgtgcacagataccggtgcacatgaagtctgatgcctcgaagtttacccacgagaaacccgaggggtactataactggcatcacggagcagtgcagtattcaggaggccggttcactatcccgacgggtgcaggcaagccgggagacagcggcagaccgatcttcgacaacaaaggacgggtggtggccatcgtcctaggaggggccaacgaaggtgcccgcacggccctctccgtggtgacgtggaacaaagacatcgtcacaaaaattacccctgagggagccgaagagtggagcctcgccctcccggtcttgtgcctgttggcaaacactacattcccctgctctcagccgccttgcacaccctgctgctacgaaaaggaaccggaaagcaccttgcgcatgcttgaggacaacgtgatgagacccggatactaccagctactaaaagcatcgctgacttgctctccccaccgccaaagacgcagtactaaggacaattttaatgtctataaagccacaagaccatatctagctcattgtcctgactgcggagaagggcattcgtgccacagccctatcgcattggagcgcatcagaaatgaagcaacggacggaacgctgaaaatccaggtctctttgcagatcgggataaagacagatgacagccacgattggaccaagctgcgctatatggatagccatacgccagcggacgcggagcgagccggattgcttgtaaggacttcagcaccgtgcacgatcaccgggaccatgggacactttattctcgcccgatgcccgaaaggagagacgctgacagtgggatttacggacagcagaaagatcagccacacatgcacacacccgttccatcatgaaccacctgtgataggtagggagaggttccactctcgaccacaacatggtaaagagttaccttgcagcacgtacgtgcagagcaccgctgccactgctgaggagatagaggtgcatatgcccccagatactcctgaccgcacgctgatgacgcagcagtctggcaacgtgaagatcacagttaatgggcagacggtgcggtacaagtgcaactgcggtggctcaaacgagggactgacaaccacagacaaagtgatcaataactgcaaaattgatcagtgccatgctgcagtcactaatcacaagaattggcaatacaactcccctttagtcccgcgcaacgctgaactcggggaccgtaaaggaaagatccacatcccattcccattggcaaacgtgacttgcagagtgccaaaagcaagaaaccctacagtaacttacggaaaaaaccaagtcaccatgctgctgtatcctgaccatccgacactcttgtcttaccgtaacatgggacaggaaccaaattaccacgaggagtgggtgacacacaagaaggaggttaccttgaccgtgcctactgagggtctggaggtcacttggggcaacaacgaaccatacaagtactggccgcagatgtctacgaacggtactgctcatggtcacccacatgagataatcttgtactattatgagctgtaccccactatgactgtagtcattgtgtcggtggcctcgttcgtgcttctgtcgatggtgggcacagcagtgggaatgtgtgtgtgcgcacggcgcagatgcattacaccatatgaattaacaccaggagccactgttcccttcctgctcagcctgctatgctgcgtcagaacgaccaaggcggccacatattacgaggctgcggcatatctatggaacgaacagcagcccctgttctggttgcaggctcttatcccgctggccgccttgatcgtcctgtgcaactgtctgaaactcttgccatgctgctgtaagaccctggcttttttagccgtaatgagcatcggtgcccacactgtgagcgcgtacgaacacgtaacagtgatcccgaacacggtgggagtaccgtataagactcttgtcaacagaccgggttacagccccatggtgttggagatggagctacaatcagtcaccttggaaccaacactgtcacttgactacatcacgtgcgagtacaaaactgtcatcccctccccgtacgtgaagtgctgtggtacagcagagtgcaaggacaagagcctaccagactacagctgcaaggtctttactggagtctacccatttatgtggggcggcgcctactgcttttgcgacgccgaaaatacgcaattgagcgaggcacatgtagagaaatctgaatcttgcaaaacagagtttgcatcggcctacagagcccacaccgcatcggcgtcggcgaagctccgcgtcctttaccaaggaaacaacattaccgtagctgcctacgctaacggtgaccatgccgtcacagtaaaggacgccaagtttgtcgtgggcccaatgtcctccgcctggacaccttttgacaacaaaatcgtggtgtacaaaggcgacgtctacaacatggactacccaccttttggcgcaggaagaccaggacaatttggtgacattcaaagtcgtacaccggaaagtaaagacgtttatgccaacactcagttggtactacagaggccagcagcaggcacggtacatgtaccatactctcaggcaccatctggcttcaagtattggctgaaggaacgaggagcatcgctacagcacacggcaccgttcggttgccagattgcgacaaacccggtaagagctgtaaattgcgctgtggggaacataccaatttccatcgacataccggatgcggcctttactagggttgtcgatgcaccctctgtaacggacatgtcatgcgaagtaccagcctgcactcactcctccgactttgggggcgtcgccatcatcaaatacacagctagcaagaaaggtaaatgtgcagtacattcgatgaccaacgccgttaccattcgagaagccgacgtagaagtagaggggaactcccagctgcaaatatccttctcaacagccctggcaagcgccgagtttcgcgtgcaagtgtgctccacacaagtacactgcgcagccgcatgccaccctccaaaggaccacatagtcaattacccagcatcacacaccacccttggggtccaggatatatccacaacggcaatgtcttgggtgcagaagattacgggaggagtaggattaattgttgctgttgctgccttaattttaattgtggtgctatgcgtgtcgtttagcaggcac

SEQ ID NO: 3 - protein, 1248 aa, Artificial Sequence
  Chikungunya (strain 37997)
MEFIPTQTFYNRRYQPRPWAPRPTIQVIRPRPRPQRQAGQLAQLISAVNKLTMRAVPQQKPRRNRKNKKQRQKKQAPQNDPKQKKQPPQKKPAQKKKKPGRRERMCMKIENDCIFEVKHEGKVMGYACLVGDKVMKPAHVKGTIDNADLAKLAFKRSSKYDLECAQIPVHMKSDASKFTHEKPEGYYNWHHGAVQYSGGRFTIPTGAGKPGDSGRPIFDNKGRVVAIVLGGANEGARTALSVVTWNKDIVTKITPEGAEEWSLALPVLCLLANTTFPCSQPPCTPCCYEKEPESTLRMLEDNVMRPGYYQLLKASLTCSPHRQRRSTKDNFNVYKATRPYLAHCPDCGEGHSCHSPIALERIRNEATDGTLKIQVSLQIGIKTDDSHDWTKLRYMDSHTPADAERAGLLVRTSAPCTITGTMGHFILARCPKGETLTVGFTDSRKISHTCTHPFHHEPPVIGRERFHSRPQHGKELPCSTYVQSTAATAEEIEVHMPPDTPDRTLMTQQSGNVKITVNGQTVRYKCNCGGSNEGLTTTDKVINNCKIDQCHAAVTNHKNWQYNSPLVPRNAELGDRKGKIHIPFPLANVTCRVPKARNPTVTYGKNQVTMLLYPDHPTLLSYRNMGQEPNYHEEWVTHKKEVTLTVPTEGLEVTWGNNEPYKYWPQMSTNGTAHGHPHEIILYYYELYPTMTVVIVSVASFVLLSMVGTAVGMCVCARRRCITPYELTPGATVPFLLSLLCCVRTTKAATYYEAAAYLWNEQQPLFWLQALIPLAALIVLCNCLKLLPCCCKTLAFLAVMSIGAHTVSAYEHVTVIPNTVGVPYKTLVNRPGYSPMVLEMELQSVTLEPTLSLDYITCEYKTVIPSPYVKCCGTAECKDKSLPDYSCKVFTGVYPFMWGGAYCFCDAENTQLSEAHVEKSESCKTEFASAYRAHTASASAKLRVLYQGNNITVAAYANGDHAVTVKDAKFVVGPMSSAWTPFDNKIVVYKGDVYNMDYPPFGAGRPGQFGDIQSRTPESKDVYANTQLVLQRPAAGTVHVPYSQAPSGFKYWLKERGASLQHTAPFGCQIATNPVRAVNCAVGNIPISIDIPDAAFTRVVDAPSVTDMSCEVPACTHSSDFGGVAIIKYTASKKGKCAVHSMTNAVTIREADVEVEGNSQLQISFSTALASAEFRVQVCSTQVHCAAACHPPKDHIVNYPASHTTLGVQDISTTAMSWVQKITGGVGLIVAVAALILIVVLCVSFSRH

SEQ ID NO: 4 - protein, 261 aa, Artificial Sequence
  Chikungunya (strain 37997) Capsid protein
MEFIPTQTFYNRRYQPRPWAPRPTIQVIRPRPRPQRQAGQLAQLISAVNKLTMRAVPQQKPRRNRKNKKQRQKKQAPQNDPKQKKQPPQKKPAQKKKKPGRRERMCMKIENDCIFEVKHEGKVMGYACLVGDKVMKPAHVKGTIDNADLAKLAFKRSSKYDLECAQIPVHMKSDASKFTHEKPEGYYNWHHGAVQYSGGRFTIPTGAGKPGDSGRPIFDNKGRVVAIVLGGANEGARTALSVVTWNKDIVTKITPEGAEEW

SEQ ID NO: 5 - DNA, 783 bp, Artificial Sequence
  Chikungunya (strain 37997) Capsid protein
atggagttcatcccgacgcaaactttctataacagaaggtaccaaccccgaccctgggccccacgccctacaattcaagtaattagacctagaccacgtccacagaggcaggctgggcaactcgcccagctgatctccgcagtcaacaaattgaccatgcgcgcggtacctcaacagaagcctcgcagaaatcggaaaaacaagaagcaaaggcagaagaagcaggcgccgcaaaacgacccaaagcaaaagaagcaaccaccacaaaagaagccggctcaaaagaagaagaaaccaggccgtagggagagaatgtgcatgaaaattgaaaatgattgcatcttcgaagtcaagcatgaaggcaaagtgatgggctacgcatgcctggtgggggataaagtaatgaaaccagcacatgtgaagggaactatcgacaatgccgatctggctaaactggcctttaagcggtcgtctaaatacgatcttgaatgtgcacagataccggtgcacatgaagtctgatgcctcgaagtttacccacgagaaacccgaggggtactataactggcatcacggagcagtgcagtattcaggaggccggttcactatcccgacgggtgcaggcaagccgggagacagcggcagaccgatcttcgacaacaaaggacgggtggtggccatcgtcctaggaggggccaacgaaggtgcccgcacggccctctccgtggtgacgtggaacaaagacatcgtcacaaaaattacccctgagggagccgaagagtgg

SEQ ID NO: 6 - protein, 64 aa, Artificial Sequence
  Chikungunya (strain 37997) Assembly protein E3
SLALPVLCLLANTTFPCSQPPCTPCCYEKEPESTLRMLEDNVMRPGYYQLLKASLTCSPHRQRR

SEQ ID NO: 7 - DNA, 194 bp, Artificial Sequence
  Chikungunya (strain 37997) Assembly protein E3
agagcctcgccctcccggtcttgtgcctgttggcaaacactacattcccctgctctcagccgccttgcacaccctgctgctacgaaaaggaaccggaaagcaccttgcgcatgcttgaggacaacgtgatgagacccggatactaccagctactaaaagcatcgctgacttgctctccccaccgccaaagacgc

SEQ ID NO: 8 - protein, 423 aa, Artificial Sequence
  Chikungunya (strain 37997) Spike glycoprotein E2
STKDNFNVYKATRPYLAHCPDCGEGHSCHSPIALERIRNEATDGTLKIQVSLQIGIKTDDSHDWTKLRYMDSHTPADAERAGLLVRTSAPCTITGTMGHFILARCPKGETLTVGFTDSRKISHTCTHPFHHEPPVIGRERFHSRPQHGKELPCSTYVQSTAATAEEIEVHMPPDTPDRTLMTQQSGNVKITVNGQTVRYKCNCGGSNEGLTTTDKVINNCKIDQCHAAVTNHKNWQYNSPLVPRNAELGDRKGKIHIPFPLANVTCRVPKARNPTVTYGKNQVTMLLYPDHPTLLSYRNMGQEPNYHEEWVTHKKEVTLTVPTEGLEVTWGNNEPYKYWPQMSTNGTAHGHPHEIILYYYELYPTMTVVIVSVASFVLLSMVGTAVGMCVCARRRCITPYELTPGATVPFLLSLLCCVRTTKA

SEQ ID NO: 9 - DNA, 1269 bp, Artificial Sequence
  Chikungunya (strain 37997) Spike glycoprotein E2
agtactaaggacaattttaatgtctataaagccacaagaccatatctagctcattgtcctgactgcggagaagggcattcgtgccacagccctatcgcattggagcgcatcagaaatgaagcaacggacggaacgctgaaaatccaggtctctttgcagatcgggataaagacagatgacagccacgattggaccaagctgcgctatatggatagccatacgccagcggacgcggagcgagccggattgcttgtaaggacttcagcaccgtgcacgatcaccgggaccatgggacactttattctcgcccgatgcccgaaaggagagacgctgacagtgggatttacggacagcagaaagatcagccacacatgcacacacccgttccatcatgaaccacctgtgataggtagggagaggttccactctcgaccacaacatggtaaagagttaccttgcagcacgtacgtgcagagcaccgctgccactgctgaggagatagaggtgcatatgcccccagatactcctgaccgcacgctgatgacgcagcagtctggcaacgtgaagatcacagttaatgggcagacggtgcggtacaagtgcaactgcggtggctcaaacgagggactgacaaccacagacaaagtgatcaataactgcaaaattgatcagtgccatgctgcagtcactaatcacaagaattggcaatacaactcccctttagtcccgcgcaacgctgaactcggggaccgtaaaggaaagatccacatcccattcccattggcaaacgtgacttgcagagtgccaaaagcaagaaaccctacagtaacttacggaaaaaaccaagtcaccatgctgctgtatcctgaccatccgacactcttgtcttaccgtaacatgggacaggaaccaaattaccacgaggagtgggtgacacacaagaaggaggttaccttgaccgtgcctactgagggtctggaggtcacttggggcaacaacgaaccatacaagtactggccgcagatgtctacgaacggtactgctcatggtcacccacatgagataatcttgtactattatgagctgtaccccactatgactgtagtcattgtgtcggtggcctcgttcgtgcttctgtcgatggtgggcacagcagtgggaatgtgtgtgtgcgcacggcgcagatgcattacaccatatgaattaacaccaggagccactgttcccttcctgctcagcctgctatgctgcgtcagaacgaccaaggcg

SEQ ID NO: 10 - protein, 61 aa, Artificial Sequence
  Chikungunya (strain 37997) 6k protein
ATYYEAAAYLWNEQQPLFWLQALIPLAALIVLCNCLKLLPCCCKTLAFLAVMSIGAHTVSA

SEQ ID NO: 11 - DNA, 183 bp, Artificial Sequence
  Chikungunya (strain 37997) 6k protein
gccacatattacgaggctgcggcatatctatggaacgaacagcagcccctgttctggttgcaggctcttatcccgctggccgccttgatcgtcctgtgcaactgtctgaaactcttgccatgctgctgtaagaccctggcttttttagccgtaatgagcatcggtgcccacactgtgagcgcg

SEQ ID NO: 12 - protein, 439 aa, Artificial Sequence
  Chikungunya (strain 37997) Spike glycoprotein E1
YEHVTVIPNTVGVPYKTLVNRPGYSPMVLEMELQSVTLEPTLSLDYITCEYKTVIPSPYVKCCGTAECKDKSLPDYSCKVFTGVYPFMWGGAYCFCDAENTQLSEAHVEKSESCKTEFASAYRAHTASASAKLRVLYQGNNITVAAYANGDHAVTVKDAKFVVGPMSSAWTPFDNKIVVYKGDVYNMDYPPFGAGRPGQFGDIQSRTPESKDVYANTQLVLQRPAAGTVHVPYSQAPSGFKYWLKERGASLQHTAPFGCQIATNPVRAVNCAVGNIPISIDIPDAAFTRVVDAPSVTDMSCEVPACTHSSDFGGVAIIKYTASKKGKCAVHSMTNAVTIREADVEVEGNSQLQISFSTALASAEFRVQVCSTQVHCAAACHPPKDHIVNYPASHTTLGVQDISTTAMSWVQKITGGVGLIVAVAALILIVVLCVSFSRH

SEQ ID NO: 13 - DNA, 1317 bp, Artificial Sequence
  Chikungunya (strain 37997) Spike glycoprotein E1
tacgaacacgtaacagtgatcccgaacacggtgggagtaccgtataagactcttgtcaacagaccgggttacagccccatggtgttggagatggagctacaatcagtcaccttggaaccaacactgtcacttgactacatcacgtgcgagtacaaaactgtcatcccctccccgtacgtgaagtgctgtggtacagcagagtgcaaggacaagagcctaccagactacagctgcaaggtctttactggagtctacccatttatgtggggcggcgcctactgcttttgcgacgccgaaaatacgcaattgagcgaggcacatgtagagaaatctgaatcttgcaaaacagagtttgcatcggcctacagagcccacaccgcatcggcgtcggcgaagctccgcgtcctttaccaaggaaacaacattaccgtagctgcctacgctaacggtgaccatgccgtcacagtaaaggacgccaagtttgtcgtgggcccaatgtcctccgcctggacaccttttgacaacaaaatcgtggtgtacaaaggcgacgtctacaacatggactacccaccttttggcgcaggaagaccaggacaatttggtgacattcaaagtcgtacaccggaaagtaaagacgtttatgccaacactcagttggtactacagaggccagcagcaggcacggtacatgtaccatactctcaggcaccatctggcttcaagtattggctgaaggaacgaggagcatcgctacagcacacggcaccgttcggttgccagattgcgacaaacccggtaagagctgtaaattgcgctgtggggaacataccaatttccatcgacataccggatgcggcctttactagggttgtcgatgcaccctctgtaacggacatgtcatgcgaagtaccagcctgcactcactcctccgactttgggggcgtcgccatcatcaaatacacagctagcaagaaaggtaaatgtgcagtacattcgatgaccaacgccgttaccattcgagaagccgacgtagaagtagaggggaactcccagctgcaaatatccttctcaacagccctggcaagcgccgagtttcgcgtgcaagtgtgctccacacaagtacactgcgcagccgcatgccaccctccaaaggaccacatagtcaattacccagcatcacacaccacccttggggtccaggatatatccacaacggcaatgtcttgggtgcagaagattacgggaggagtaggattaattgttgctgttgctgccttaattttaattgtggtgctatgcgtgtcgtttagcaggcac

(19) United States
(12) Patent Application Publication
(10) Pub. No.: US 2022/0409717 A1
(43) Pub. Date: Dec. 29, 2022

(54) CHIKUNGUNYA VIRUS-LIKE PARTICLE VACCINE AND METHODS OF USING THE SAME

(71) Applicant: Emergent Travel Health Inc., Redwood City, CA (US)

(72) Inventors: Jeffery L. ALEXANDER, San Diego, CA (US); Sean Robert BENNETT, Charlottesville, CA (US); Jonathan Fowler SMITH, Redwood City, CA (US)

(73) Assignee: Emergent Travel Health Inc., Redwood City, CA (US)

(21) Appl. No.: 17/771,782

(22) PCT Filed: Oct. 26, 2020
(86) PCT No.: PCT/US2020/057361

§ 371 Date: Apr. 25, 2022

Related U.S. Application Data
(60) Provisional application No. 62/993,563, filed on Mar. 23, 2020.
(60) Provisional application No. 62/926,357, filed on Oct. 25, 2019.

(51) Int. Cl. A61K 39/12; A61K 9/00; A61P 31/14

(52) U.S. Cl. CPC A61K 39/12; A61K 9/0019; A61P 31/14; A61K 2039/5258

(57) ABSTRACT

The present disclosure is directed to improved virus-like particle (VLP) compositions and vaccines for use in inducing an immune response and/or protective immunity against a Chikungunya virus (CHIKV) infection in a subject, e.g., by inducing a neutralizing antibody response against CHIKV in a subject within 7 days after administration of a single dose of the composition or vaccine.

### CROSS REFERENCE TO RELATED APPLICATION

This application claims priority to U.S. Provisional Appl. No. 62/926,357 filed Oct. 25, 2019, and U.S. Provisional Appl. No. 62/993,563 filed Mar. 23, 2020, the content of each is hereby incorporated by reference in their entireties.

### REFERENCE TO SEQUENCE LISTING SUBMITTED ELECTRONICALLY

The content of the electronically submitted sequence listing in ASCII text file (Name: 2479_215PC02_Seqlisting_ST25; Size: 44,121 bytes; and Date of Creation: Oct. 22, 2020) filed with the application is incorporated herein by reference in its entirety.

### BACKGROUND

Chikungunya virus (CHIKV) is a mosquito-borne alphavirus in the family Togaviridae, which was first identified in Tanzania in 1952. Phylogenetic analysis of CHIKV showed that there are three genotypes: Asian, East/Central/South African and West African. Infection by this virus causes human disease that is characterized by sudden fever onset, muscle pain, headaches, rashes, and inflammation in joints. While the fever is typically short term, severe arthritic symptoms can persist for years. Chronicity of the inflammatory condition is associated with musculoskeletal disorders such as distal fasciitis and proximal tendonitis, which require both pharmacological and physical therapies. Elderly and persons with comorbidities such as neurological, respiratory or cardiovascular diseases are at higher risk for more severe disease.

CHIKV has infected millions of people in Africa, Europe, and Asia since its re-emergence in Kenya in 2004. Some reports suggest that up to 40% of the global population is at risk for CHIKV exposure. The evolution and spread of the virus into new geographic areas, and the disease severity present a serious public health threat in the absence of a vaccines or anti-viral therapies. No specific treatment or vaccine is currently approved for this disease. The existing paradigm is primarily directed at relieving disease symptoms such as providing commonly used medications including antipyretics, analgesics and fluids.

### BRIEF SUMMARY

Certain aspects of the disclosure are directed to a composition or vaccine comprising: (a) a virus-like particle (VLP) which comprises a capsid protein and an envelope protein derived from Chikungunya virus (CHIKV); (b) an aluminum hydroxide adjuvant (e.g., aluminum hydroxide gel (e.g., Alhydrogel®)); (c) a sugar; (d) a buffer and/or pH modifier; and (e) a carrier.

Certain aspects of the disclosure are directed to a composition or vaccine comprising a virus-like particle (VLP) which comprises a capsid protein and an envelope protein derived from Chikungunya virus (CHIKV), wherein the composition or vaccine induces a neutralizing antibody response against CHIKV in a subject within 7 days after administration of a single dose of the composition or vaccine to the subject. Certain aspects of the disclosure are directed to a composition or vaccine comprising a virus-like particle (VLP) which comprises a capsid protein and an envelope protein derived from Chikungunya virus (CHIKV), wherein the composition or vaccine induces a neutralizing antibody response against CHIKV in a subject, wherein the neutralizing antibody response against CHIKV persists for at least 28 days, at least 6 months, at least 1 year, or at least 2 years after the single dose is administered. In some aspects, the composition or vaccine further comprises an adjuvant, e.g., an aluminum hydroxide adjuvant (e.g., aluminum hydroxide gel (e.g., Alhydrogel®)).

In some aspects, the capsid protein is a CHIKV C protein. In some aspects, the envelope protein is a CHIKV protein selected from the group consisting of E3, E2, 6K, E1, and any combination thereof. In some aspects, the VLP comprises CHIKV envelope proteins E2 and E1. In some aspects, the VLP comprises CHIKV C, E2, and E1 proteins. In some aspects, the CHIKV C, E2, and E1 proteins are at least 95% of the VLP proteins in the composition or vaccine. In some aspects, the VLP does not comprise CHIKV E3 and/or 6K. In some aspects, the capsid and envelope proteins are derived CHIKV strain 37997.

In certain aspects, the composition or vaccine is a suspension suitable for intramuscular injection. In some aspects, a single dose of the composition or vaccine comprises about 6 μg to 200 μg VLP or about 20 μg to 100 μg VLP (e.g., about 40 μg). In some aspects, a single dose of the composition or vaccine comprises about 25 μg/mL to 75 μg/mL VLP (e.g., about 50 μg/mL VLP). In some aspects, a single dose of the composition or vaccine comprises an adjuvant, e.g., an aluminum hydroxide adjuvant (e.g., aluminum hydroxide gel (e.g., Alhydrogel®)) in an amount of about 30 μg to 3000 μg or about 100 μg to 500 μg (e.g., about 300 μg). In some aspects, a single dose of the composition or vaccine comprises about 250 μg/mL to 625 μg/mL aluminum hydroxide gel (e.g., Alhydrogel®) (e.g., about 375 μg/mL aluminum hydroxide gel (e.g., Alhydrogel)®). In some aspects, a single dose of the composition or vaccine comprises a volume of about 0.5 mL to 2 mL or about 0.5 mL to 1 mL (e.g., about 0.8 mL).

In certain aspects, the composition or vaccine comprises a sugar (e.g., sucrose, lactose, or any combination thereof). In certain aspects, the composition or vaccine comprises a buffer and/or pH modifier (e.g., potassium phosphate monobasic, potassium phosphate dibasic, sodium citrate dehydrate, or any combination thereof). In certain aspects, the composition or vaccine does not comprise a preservative or antibiotic.

In certain aspects, a single dose of the composition or vaccine comprises: (a) about 6 μg to 60 μg of the VLP and (b) about 200 μg to 500 μg of a/the aluminum hydroxide, wherein the single dose volume is between about 0.5 mL to 1.0 mL.

In certain aspects, a single dose of the composition or vaccine comprises: (a) about 6 μg to 60 μg of the VLP; (b) about 200 μg to 500 μg of the aluminum hydroxide; (c) about 20 mg to 80 mg of sucrose; (d) about 0.2 mg to 1.0 mg of potassium phosphate monobasic; (e) about 0.2 mg to 1.5 mg of potassium phosphate diabasic; (f) about 2 mg to 10 mg sodium citrate dehydrate; and (g) water, wherein the single dose volume is between about 0.5 mL to 1.0 mL (e.g., 0.8 mL).

In certain aspects, the composition or vaccine comprises a VLP that induces antibodies against homologous or heterologous strains of CHIKV. In some aspects, the VLP induces antibodies against one or more CHIKV lineages selected from the group consisting of East/Central/South African (ECSA), West African and Asian.

In certain aspects, the composition or vaccine induces a neutralizing antibody response against CHIKV in a subject within 7 days after administration of a/the single dose of the composition or vaccine to the subject. In some aspects, the neutralizing antibody response against CHIKV is sustained for at least 28 days after a/the single dose is administered. In some aspects, the neutralizing antibody response against CHIKV is sustained for at least six months after the single dose is administered. In some aspects, the neutralizing antibody response against CHIKV is sustained for at least one year, at least 18 months, or at least two years after the single dose is administered. In some aspects, the serum neutralizing antibody titer is ≥30 or ≥40 within 7 days after administration of a/the single dose of the composition or vaccine.

Certain aspects of the disclosure are directed to a pre-filled syringe comprising a single dose of a composition or vaccine disclosed herein.

Certain aspects of the disclosure are directed to a method of inducing an immune response and/or protective immunity against Chikungunya virus (CHIKV) in a subject, comprising administering to the subject the composition or vaccine disclosed herein.

Certain aspects of the disclosure are directed to a method of inducing a neutralizing antibody response to Chikungunya virus (CHIKV) in a subject, comprising administering to the subject the composition or vaccine disclosed herein.

Certain aspects of the disclosure are directed to a method for treating, preventing, or reducing the risk of a Chikungunya infection in a subject, comprising administering to the subject an effective amount of the composition or vaccine disclosed herein.

Certain aspects of the disclosure are directed to a method of vaccinating a subject against a CHIKV infection, comprising administering to the subject an effective amount of the composition or vaccine disclosed herein.

Certain aspects of the disclosure are directed to a method of eliciting an immune response and/or protective immunity against Chikungunya virus (CHIKV) in a subject comprising: administering to the subject a composition or vaccine comprising (a) virus-like particles (VLPs) which comprise a capsid protein and an envelope protein derived from CHIKV and (b) an adjuvant, wherein a single dose of the composition or vaccine comprises about 6 μg to 200 μg VLPs, and wherein the administration of the single dose of the composition or vaccine elicits production of CHIKV-specific neutralizing antibodies in the subject within 7 days of administration.

Certain aspects of the disclosure are directed to a method of inducing protective immunity against Chikungunya virus (CHIKV) in a population comprising: administering to the population a composition or vaccine comprising (a) virus-like particles (VLPs) which comprise a capsid protein and an envelope protein derived from CHIKV and (b) an adjuvant, wherein a single dose of the composition or vaccine comprises about 6 μg to 200 μg VLPs, and wherein at least 60% of the population produces a neutralizing antibody response against CHIKV within 7 days after administration of the single dose of the composition or vaccine. In some aspects, at least 50%, at least 60%, at least 70%, at least 80%, at least 90%, at least 95%, at least 96%, at least 97%, or at least 98% of the population develops a serum neutralizing antibody titer≥30 or ≥40 within 7 days after administration of a single dose of the composition or vaccine. In some aspects, at least 40%, at least 50%, or at least 60% of the population maintains a serum neutralizing antibody titer≥30 or ≥40 for at least 28 days, at least 6 months, at least one year, or at least two years after administration of a single dose of the composition or vaccine.

In some aspects, the neutralizing antibody response vaccinates the subject against multiple serotypes and/or genotypes of CHIKV. In some aspects, the neutralizing antibody response against CHIKV is sustained for at least 28 days, at least 6 months, at least 1 year, or at least 2 years after the single dose is administered. In some aspects, a second dose of the composition or vaccine is administered to the subject at least 28 days, at least 6 months, or at least 1 year after the single dose. In some aspects, one or more booster dose(s) of the composition or vaccine can be administered throughout lifetime of the subject, e.g., at least 28 days, at least 6 months, at least 1 year, at least 2 years, at least 5 years, at least 10 years, or 20 years or more after the single dose. In some aspects, the neutralizing antibody response against CHIKV is sustained for at least six months after administration of the second dose. In some aspects, the neutralizing antibody response against CHIKV is sustained for at least one year after administration of the second dose. In some aspects, the neutralizing antibody response against CHIKV (e.g., after a single dose or one or more doses) is sustained for at least 30 months, at least two years, at least 3 years, at least 4 years, at least 5 years, at least 10 years, or 20 years or more after administration of the second dose. In some aspects, the neutralizing antibody response against CHIKV is sustained for the lifetime of the subject. In some aspects, at least 60%, at least 70%, at least 80%, at least 90%, at least 95%, or at least 98% of the subjects produced a neutralizing antibody response to CHIKV 7 days after administration of the composition or vaccine. In some aspects, the seroresponse rate is at least 60%, at least 70%, at least 80%, at least 90%, or at least 95% 7 days, 28 days, one year, or two years after administration of the composition or vaccine. In some aspects, the serum neutralizing antibody titer is ≥30 or ≥40 or ≥50.

In some aspects, the composition or vaccine is administered intramuscularly.

In some aspects, the composition or vaccine is administered in an effective amount.

In some aspects, the subject is a human. In some aspects, the subject had no exposure to a CHIKV antigen or a CHIKV vaccine prior to administration of the composition or vaccine. In some aspects, the subject has no detectable CHIKV antibody titer prior to administration of the composition or vaccine. In some aspects, the subject has a detectable CHIKV antibody titer≤5 or ≤10 prior to administration of the composition or vaccine.

### BRIEF DESCRIPTION OF THE DRAWINGS/FIGURES

FIG. 1 shows a schematic of the CHIKV genome.

FIG. 2 shows a SDS-PAGE gel of CHIKV VLPs under reducing and denaturing conditions.

FIGS. 3A-3D are mass spectrum plots from CHIKV VLP proteins (A) Capsid, (B) E2, and (C) E1 compared to (D) control HSP70 1A after MALDI-TOF analysis.

FIG. 4 shows a RP-HPLC chromatogram of CHIKV VLP.

FIG. 5 shows a SDS-PAGE gel of peaks 1, 2, 3, that were isolated from RP-HPLC.

FIGS. 6A-6B show (A) a transmission electron microscopy image of CHIKV VLPs, and (B) cryoelectron microscopy image of CHIKV VLPs.

FIG. 7 shows an optical density plot as a function of the CHIKV VLP concentration.

FIG. 8 shows the serum neutralizing antibody response in Cynomolgus macaques after vaccination with 1.25 μg CHIKV VLP+Alum, 6 μg CHIKV VLP+Alum, 20 μg CHIKV VLP only, and 20 μg CHIKV VLP+Alum compared to Alum only and prior to challenge.

FIGS. 9A-9B show the (A) peak viremia in plasma and (B) AUC in plasma results of a plaque assay using serum isolated from challenged macaques administered 1.25 μg CHIKV VLP+Alum, 6 μg CHIKV VLP+Alum, 20 μg CHIKV VLP only, and 20 μg CHIKV VLP+Alum compared to Alum only.

FIGS. 10A-10B show the joint pathology scores for (A) adjuvant v. vaccinated and (B) 1.25 μg CHIKV VLP+Alum, 6 μg CHIKV VLP+Alum, 20 μg CHIKV VLP only, and 20 μg CHIKV VLP+Alum compared to Alum only in challenged macaques.

FIGS. 11A-11D show the amount of viral RNA amplified from challenged macaques administered 1.25 μg CHIKV VLP+Alum, 6 μg CHIKV VLP+Alum, 20 μg CHIKV VLP only, and 20 μg CHIKV VLP+Alum compared to Alum only. Samples were collected from the (12A) ankle-joint capillaries and cartilage, (12B) right quad muscle with tendon, (12C) stifle-joint capillaries and cartilage, and (12D) the wrist-joint capillaries and cartilage.

FIG. 12 shows the duration of immune response in CHIKV-naïve subjects that were administered two doses of CHIKV VLP.

FIG. 13 shows the dose and schedule design of the phase 2 study CHIKV VLP vaccine. VLP doses are in μg; A=alum; PLA=placebo.

FIG. 14 shows the quantification of the geometric mean titer of neutralizing antibodies post-vaccination (days) of for Groups 1-8 post-vaccination with CHIKV VLP vaccine.

FIG. 15 shows the mean titers of antibodies against chikungunya through day 760 post-vaccination for Groups 1-9 (see Table 7).

FIG. 16 shows the reverse cumulative distribution of titers through day 365 post-vaccination for Groups 1-8 (see Table 7).

FIG. 17 shows Geometric Mean Titer (GMT) data (see Table 8) through day 760 for Groups 1-8 (see Table 7).

FIGS. 18A-18B show the percent of subjects per Group (see Table 7) with anti-CHIKV neutralization titer (NT80)≥15 and ≥40, respectively.

### DETAILED DESCRIPTION

The present disclosure is directed to improved virus-like particle (VLP) compositions and vaccines for use in inducing an immune response and/or protective immunity against a Chikungunya virus (CHIKV) infection in a subject, e.g., by inducing a neutralizing antibody response against CHIKV in a subject within 7 days after administration of a single dose of the composition or vaccine and/or wherein the neutralizing antibody response against CHIKV persists for at least 28 days, at least 6 months, at least 1 year, or at least 2 years after a single dose is administered.

### Definitions

Unless defined otherwise, all technical and scientific terms used herein have the same meaning as commonly understood by one of ordinary skill in the art pertinent to the methods and compositions described. The definitions provided herein are to facilitate understanding of certain terms used frequently herein.

Unless specifically stated or obvious from context, as used herein, the term “or” is understood to be inclusive. Unless specifically stated or obvious from context, as used herein, the terms “a”, “an”, and “the” are understood to be singular or plural.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used herein, “agent” means any small molecule chemical compound, antibody, nucleic acid molecule, or polypeptide, or fragments thereof.

As used herein, the term “adjuvant” is meant to refer to a compound that, when used in combination with a specific immunogen in a formulation (e.g., a vaccine), will augment, alter or modify the resultant immune response. In certain embodiments, the adjuvant is used in combination with a VLP. Modification of the immune response includes intensification or broadening the specificity of either or both antibody and cellular immune responses. Modification of the immune response can also mean decreasing or suppressing certain antigen-specific immune responses. In certain aspects, the adjuvant is an aluminum hydroxide adjuvant (e.g., aluminum hydroxide gel (e.g., Alhydrogel®) 2%). Alhydrogel® is an aluminum hydroxide (referred to as alum) wet gel suspension.

As used herein “alphavirus” is meant to refer to RNA-containing viruses that belong to the Togaviridae family of viruses. Exemplary togaviruses include but are not limited to EEEV, WEEV, VEEV, SFV, CHIKV, O'nyong-nyong virus, Sindbis virus, Mayaro virus, Ross River virus, Barmah Forest virus, and Ockelbo virus.

The terms “Chikungunya Virus” and “CHIKV” are used interchangeably herein.

As used herein, “Chikungunya virus structural protein” means a polypeptide or fragment thereof having at least about 85% amino acid sequence identity to a naturally occurring Chikungunya virus capsid or envelope protein. In some aspects, the amino acid sequence identity is at least about 90%, 95%, 98%, 99%, or more.

As used herein, “ameliorate” means decrease, suppress, attenuate, diminish, arrest, or stabilize the development or progression of a disease or a symptom thereof.

As used herein in reference to a polypeptide sequence or polynucleotide sequence, “alteration” means a change in an amino acid or nucleotide at a specified position in a polypeptide sequence or polynucleotide sequence. As used herein, an alteration includes a substitution, deletion, or insertion of an amino acid or nucleotide at a specified position of a polypeptide or polynucleotide. In some aspects, an alteration in an alphavirus capsid protein nuclear localization signal includes substitution of a charged amino acid (e.g., lysine or arginine) with an uncharged amino acid (e.g., alanine or asparagine, or any amino acid except a basic charged amino acid such as lysine or arginine).

As used herein in reference to the expression levels or activity of a gene or polypeptide, “alteration” means a change (increase or decrease) to the expression levels or activity of a gene or polypeptide as detected by standard art known methods, such as those described herein. As used herein, an alteration includes a 10%, 25%, 50%, 75%, 100% or greater change in expression levels. An alteration includes a 10-, 20-, 50-, 70-, 80-, 90-, 100-, 200-, 500-, 1000-fold or greater change in expression levels.

As used herein, “analog” means a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog can include an unnatural amino acid.

As used herein “boosting” or “booster” dose or immunization refers to a re-exposure to the immunizing antigen after an earlier dose, e.g., a priming dose.

“Detect” refers to identifying the presence, absence or amount of the analyte to be detected.

By “disease” is meant any condition or disorder that damages or interferes with the normal function of a cell, tissue, or organ. Examples of diseases include viral infections including CHIKV.

As used herein, “effective amount” of a composition or vaccine means the amount of the composition or vaccine needed to prevent, reduce the likelihood of, or ameliorate the symptoms of a CHIKV infection relative to an untreated patient. The effective amount of a composition or vaccine of the disclosure used for prevention, reduction in likelihood, or treatment of CHIKV infection can vary depending upon the manner of administration, the age, body weight, and general health of the subject. In certain aspects, the effective amount is an amount of the composition or vaccine of the disclosure that provides a neutralizing antibody response and/or protection against CHIKV within 7 days.

As used herein, “fragment” means a portion of a polypeptide or nucleic acid molecule. This portion contains, for example, at least 10%, 20%, 30%, 40%, 50%, 60%, 70%, 80%, 90%, or 95% of the entire length of the reference nucleic acid molecule or polypeptide. A fragment can contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which can be Watson-Crick, Hoogsteen or reversed Hoogsteen hydrogen bonding, between complementary nucleobases. For example, adenine and thymine are complementary nucleobases that pair through the formation of hydrogen bonds.

As used herein, “inducing immunity” is meant to refer to any immune response generated against an antigen. In some aspects, immunity is mediated by antibodies against an infectious agent, which is exhibited by a vertebrate (e.g., a human), that prevents or ameliorates an infection or reduces at least one symptom thereof. VLPs of the present disclosure can stimulate the production of antibodies that, for example, neutralize infectious agents, block infectious agents from entering cells, block replication of infectious agents, and/or protect host cells from infection and destruction. The term can also refer to an immune response that is mediated by T-lymphocytes and/or other white blood cells against an infectious agent, exhibited by a vertebrate (e.g., a human), that prevents or ameliorates an infection, for example CHIKV infection, or reduces at least one symptom thereof.

As used herein, “neutralizing antibody response” refers to induction of an antibody that binds to an antigen of an infectious body (e.g., CHIKV) wherein the antibody has a neutralizing or inhibiting effect on the infectivity of the infectious body on cells. In certain aspects, the neutralizing antibody response can be determined by measuring serum neutralizing antibody (SNA) titer. In certain aspects, the SNA titer can be determined via characterization of a reduction of infectivity in the presence of serum (e.g., NT80 Serum Neutralizing Antibodies by Luciferase Assay using a luciferase vector, and/or plaque reduction assay). In some aspects, the CHIKV neutralizing antibody titer 80 (NT80) is the reciprocal of the serum dilution that provides about 80% protection of Vero cells from CHIKV-Luc infection or an 80% reduction of luciferase activity compared to virus only control. In certain aspects, the NT80 titer of 20, 25, 30, 35, 40, 45, or 50 or greater indicates protections against one or more or all lineages of CHIKV.

As used herein, “obtaining” as in “obtaining an agent” includes synthesizing, purchasing, or otherwise acquiring the agent.

As used herein, “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “population” refers to a pool or group of individuals from which a statistical sample is drawn. In some aspects of the disclosure, a target population for vaccination can be a population that has not been primed from chikungunya virus and is naïve (e.g., against one or more strains of CHIKV). In other aspects, a target population can be a population that has previously been exposed chikungunya infection or vaccination (e.g., against one or more strains of CHIKV). The population, e.g., the target population, can be include individuals of any age and ethnicity.

By “reduces” is meant a negative alteration of at least 10%, 25%, 50%, 75%, 85%, 95%, or 100%.

By “reference” is meant a standard or control condition.

A “reference sequence” refers to a defined sequence used as a basis for sequence comparison. A reference sequence can be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, and about 35 amino acids, about 50 amino acids, or about 100 amino acids. For nucleic acids, the length of the reference nucleic acid sequence will generally be at least about 50 nucleotides, at least about 60 nucleotides, at least about 75 nucleotides, and about 100 nucleotides or about 300 nucleotides or any integer thereabout or there between.

As used herein, “specifically binds” can refer to a compound or antibody that recognizes and binds a polypeptide of the disclosure, but which does not substantially recognize and bind other molecules in a sample, for example, a biological sample, which naturally includes a polypeptide of the disclosure.

Nucleic acid molecules useful in the methods of the disclosure include any nucleic acid molecule that encodes a polypeptide of the disclosure or a fragment thereof. Such nucleic acid molecules need not always be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the disclosure include any nucleic acid molecule that encodes a polypeptide of the disclosure or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). In certain aspects, a sequence is at least 60%, at least 80%, at least 85%, at least 90%, at least 95%, at least 98% or at least 99% identical at the amino acid level or nucleic acid to the sequence used for comparison.

Sequence identity can be measured using sequence analysis software (for example, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program can be used, with a probability score between e-3 and e-100 indicating a closely related sequence.

By “structural polyprotein” is meant a composite amino acid molecule comprising at least two separable polypeptides that contribute to a viral capsid or envelope. In one aspect, the polypeptides are susceptible to cleavage with a viral enzyme (e.g., capsid autoproteinase and signalases).

By “subject” is meant a mammal, including, but not limited to, a human or non-human mammal, such as a bovine, equine, canine, ovine, or feline. In certain aspects, the subject is a human. The term “subject” can be used herein interchangeably with “individual” or “patient.” A subject can be afflicted with or is susceptible to a disease or disorder but may or may not display symptoms of the disease or disorder.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated. In some aspects, a treatment disclosed herein can be used prophylactically for preventing or reducing the risk of the relevant disease or condition.

As used herein, the phrase “therapeutically effective” is intended to qualify the amount of a composition (e.g., a vaccine), or the combined amount of active ingredients in the case of combination therapy. This amount or combined amount will achieve the goal of treating, preventing, or reducing the risk of the relevant disease or condition.

As used herein, the term “vaccine” refers to a composition to be used in generating an immune response. In particular aspects, a vaccine of the disclosure contains VLPs, DNAs, or other gene-based vaccine vectors in a form that is capable of being administered to a subject and which induces a protective immune response sufficient to induce immunity to prevent and/or ameliorate an infection and/or to reduce at least one symptom of an infection and/or to enhance the efficacy of another dose of VLPs or DNA vaccines. Typically, the vaccine comprises a pharmaceutically acceptable excipient, such as conventional saline or buffered aqueous solution medium in which the composition of the present disclosure is suspended or dissolved. In this form, the composition of the present disclosure can be used conveniently to prevent, ameliorate, or otherwise treat an infection. Upon introduction into a host, the vaccine induces an immune response including, but not limited to, the production of antibodies and/or cytokines and/or the activation of cytotoxic T cells, antigen presenting cells, helper T cells, dendritic cells and/or other cellular responses. In certain aspects, a vaccine can also be a protein. For example, recombinant proteins have been produced by genetically engineering cells to produce one or more foreign genes, which in turn produce proteins that serve as the immunogen.

As used herein, the “virus-like particle” (VLP) refers to a structure that in at least one attribute resembles a virus, but which has not been demonstrated to be infectious. Virus-like particles in accordance with the disclosure do not carry genetic information encoding the proteins of the virus-like particles. In general, virus-like particles lack a viral genome and, therefore, are noninfectious.

The recitation of an embodiment for a variable or aspect herein includes that embodiment as any single embodiment or in combination with any other embodiments or portions thereof disclosed herein.

Any compositions or methods provided herein can be combined with one or more of any of the other compositions and methods provided herein.

### Virus Like Particles (VLPs)

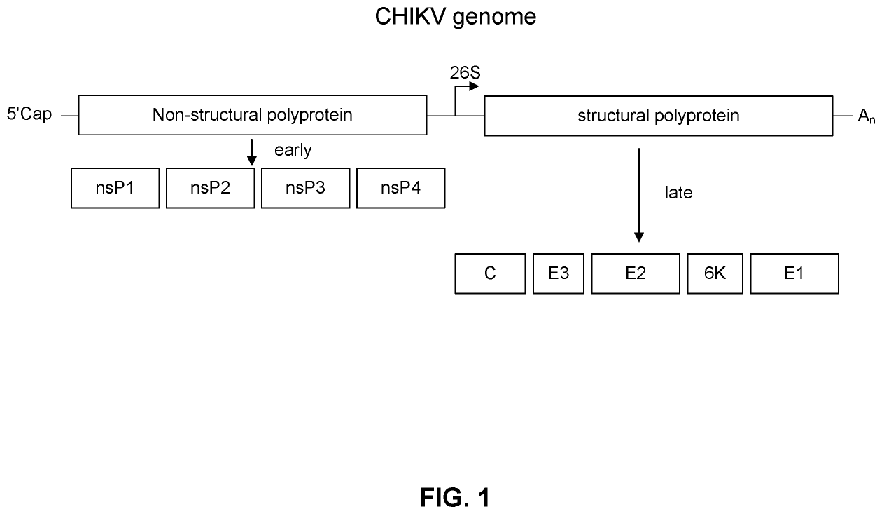

Certain aspects of the disclosure are directed to virus like particles (VLPs) comprised of structural proteins derived from chikungunya virus (CHIKV), e.g., Senegal strain 37997. CHIKV is a member of the Alphavirus genus (RNA (+) genome) and has three lineages: East/Central/South African (ECSA), West African and Asian. The CHIKV genome includes 4 non-structural proteins and 5 structural proteins (FIG. 1), and the wild-type CHIKV virion includes C, E1, and E2 proteins.

In certain aspects, the CHIKV VLP includes three recombinant CHIKV structural proteins: Capsid/core (about 35 kDa), Envelope 1 (E1, about 55 kDa), and Envelope 2 (E2, about 50 kDa). The three structural proteins can assemble to form CHIKV VLPs.

The structure of the CHIKV VLP capsid core includes hexamers around each 2-fold axis and pentamers at each 5-fold vertex of the nuclear capsid. The capsid core is separated from the E1 and E2 proteins by a ˜15 Å-wide lipid membrane. The membrane is traversed by pairs of α helices representing the E1 and E2 carboxyterminal regions. Cryoelectron microscopy has shown the trimeric appearance of individual spikes that are composed of three heterodimers of E1 and E2. β-strands in the E1 domain III can be observed. Sun, S., et al. eLife 2013; 2:e00435.

Applicant has analyzed recombinant CHIKV VLPs produced by methods of the current disclosure by negative stain and transmission electronic microscopy and by cryoelectron microscopy analyses. By negative stain, CHIKV VLPs appear as approximately spherical particles with a regular surface structure, and in certain aspects, fully formed mature VLPs are approximately 65 nm. In some aspects, the VLPs are about 60 nm to 70 nm. In certain aspects, the VLP size can be confirmed by microscopy and/or dynamic light scattering (DLS).

In certain aspects of the disclosure, the CHIKV VLPs comprise one or more CHIKV VLP proteins. In certain aspects, a VLP comprises at least three recombinant CHIKV structural proteins, e.g., a capsid core protein (C), an Envelope 1 protein (E1), and an Envelope 2 protein (E2). In some aspects, the VLP can further comprise an Envelope 3 protein (E3) and/or a 6k protein (6K).

In some aspects, the capsid protein is a CHIKV C protein.

In some aspects, the envelope protein is a CHIKV protein selected from the group consisting of E3, E2, 6K, E1, and any combination thereof. In some aspects, the CHIKV envelope proteins E2 and E1.

Some aspects of the disclosure are directed to a VLP comprising CHIKV C, E2, and E1 proteins. In certain aspects, the VLP does not comprise CHIKV E3 and/or 6K.

In certain aspects, the capsid and envelope proteins are derived from CHIKV strain 37997. Table 1 below lists the amino acid sequences corresponding to CHIKV (strain 37997) structural proteins C, E3, E2, 6K, and E1 and the nucleic acid sequences encoding the same.

TABLE 1
CHIKV VLP (strain 37997) Sequences

| SEQ ID | | Short |
|---|---|---|
| NO | Sequence | Description |
| 4 | MEFIPTQTFYNRRYQPRPWAPRPTIQVIRPRPRPQRQAG | CHIKV (strain |
| | QLAQLISAVNKLTMRAVPQQKPRRNRKNKKQRQKKQA | 37997) Capsid |
| | PQNDPKQKKQPPQKKPAQKKKKPGRRERMCMKIENDC | protein-amino acid |
| | IFEVKHEGKVMGYACLVGDKVMKPAHVKGTIDNADLA | sequence |
| | KLAFKRSSKYDLECAQIPVHMKSDASKFTHEKPEGYYN | |
| | WHHGAVQYSGGRFTIPTGAGKPGDSGRPIFDNKGRVVA | |
| | IVLGGANEGARTALSVVTWNKDIVTKITPEGAEEW | |
| 5 | atggagttcatcccgacgcaaactttctataacagaaggtaccaaccccgaccctgggc | CHIKV (strain |
| | cccacgccctacaattcaagtaattagacctagaccacgtccacagaggcaggctggg | 37997) Capsid |
| | caactcgcccagctgatctccgcagtcaacaaattgaccatgcgcgcggtacctcaaca | protein-nucleic |
| | gaagcctcgcagaaatcggaaaaacaagaagcaaaggcagaagaagcaggcgccg | acid sequence |
| | caaaacgacccaaagcaaaagaagcaaccaccacaaaagaagccggctcaaaagaa | |
| | gaagaaaccaggccgtagggagagaatgtgcatgaaaattgaaaatgattgcatcttcg | |
| | aagtcaagcatgaaggcaaagtgatgggctacgcatgcctggtgggggataaagtaat | |
| | gaaaccagcacatgtgaagggaactatcgacaatgccgatctggctaaactggccttta | |
| | agcggtcgtctaaatacgatcttgaatgtgcacagataccggtgcacatgaagtctgatg | |
| | cctcgaagtttacccacgagaaacccgaggggtactataactggcatcacggagcagt | |
| | gcagtattcaggaggccggttcactatcccgacgggtgcaggcaagccgggagacag | |
| | cggcagaccgatcttcgacaacaaaggacgggtggtggccatcgtcctaggaggggc | |
| | caacgaaggtgcccgcacggccctctccgtggtgacgtggaacaaagacatcgtcaca | |
| | aaaattacccctgagggagccgaagagtgg | |
| 6 | SLALPVLCLLANTTFPCSQPPCTPCCYEKEPESTLRMLE | CHIKV (strain |
| | DNVMRPGYYQLLKASLTCSPHRQRR | 37997) Assembly |
| | | protein E3-amino |
| | | acid sequence |
| 7 | agagcctcgccctcccggtcttgtgcctgttggcaaacactacattcccctgctctcagcc | CHIKV (strain |
| | gccttgcacaccctgctgctacgaaaaggaaccggaaagcaccttgcgcatgcttgag | 37997) Assembly |
| | gacaacgtgatgagacccggatactaccagctactaaaagcatcgctgacttgctctccc | protein E3-nucleic |
| | caccgccaaagacgc | acid sequence |
| 8 | STKDNFNVYKATRPYLAHCPDCGEGHSCHSPIALERIRN | CHIKV (strain |
| | EATDGTLKIQVSLQIGIKTDDSHDWTKLRYMDSHTPAD | 37997) Spike |
| | AERAGLLVRTSAPCTITGTMGHFILARCPKGETLTVGFT | glycoprotein E2- |
| | DSRKISHTCTHPFHHEPPVIGRERFHSRPQHGKELPCSTY | amino acid |
| | VQSTAATAEEIEVHMPPDTPDRTLMTQQSGNVKITVNG | sequence |
| | QTVRYKCNCGGSNEGLTTTDKVINNCKIDQCHAAVTN | |
| | HKNWQYNSPLVPRNAELGDRKGKIHIPFPLANVTCRVP | |
| | KARNPTVTYGKNQVTMLLYPDHPTLLSYRNMGQEPNY | |
| | HEEWVTHKKEVTLTVPTEGLEVTWGNNEPYKYWPQM | |
| | STNGTAHGHPHEIILYYYELYPTMTVVIVSVASFVLLSM | |
| | VGTAVGMCVCARRRCITPYELTPGATVPFLLSLLCCVR | |
| | TTKA | |
| 9 | agtactaaggacaattttaatgtctataaagccacaagaccatatctagctcattgtcctga | CHIKV (strain |
| | ctgcggagaagggcattcgtgccacagccctatcgcattggagcgcatcagaaatgaa | 37997) Spike |
| | gcaacggacggaacgctgaaaatccaggtctctttgcagatcgggataaagacagatg | glycoprotein E2- |
| | acagccacgattggaccaagctgcgctatatggatagccatacgccagcggacgcgg | nucleic acid |
| | agcgagccggattgcttgtaaggacttcagcaccgtgcacgatcaccgggaccatggg | sequence |
| | acactttattctcgcccgatgcccgaaaggagagacgctgacagtgggatttacggaca | |
| | gcagaaagatcagccacacatgcacacacccgttccatcatgaaccacctgtgataggt | |
| | agggagaggttccactctcgaccacaacatggtaaagagttaccttgcagcacgtacgt | |
| | gcagagcaccgctgccactgctgaggagatagaggtgcatatgcccccagatactcct | |
| | gaccgcacgctgatgacgcagcagtctggcaacgtgaagatcacagttaatgggcaga | |
| | cggtgcggtacaagtgcaactgcggtggctcaaacgagggactgacaaccacagaca | |
| | aagtgatcaataactgcaaaattgatcagtgccatgctgcagtcactaatcacaagaattg | |
| | gcaatacaactcccctttagtcccgcgcaacgctgaactcggggaccgtaaaggaaag | |
| | atccacatcccattcccattggcaaacgtgacttgcagagtgccaaaagcaagaaaccc | |
| | tacagtaacttacggaaaaaaccaagtcaccatgctgctgtatcctgaccatccgacact | |
| | cttgtcttaccgtaacatgggacaggaaccaaattaccacgaggagtgggtgacacaca | |
| | agaaggaggttaccttgaccgtgcctactgagggtctggaggtcacttggggcaacaac | |
| | gaaccatacaagtactggccgcagatgtctacgaacggtactgctcatggtcacccaca | |
| | tgagataatcttgtactattatgagctgtaccccactatgactgtagtcattgtgtcggtggc | |
| | ctcgttcgtgcttctgtcgatggtgggcacagcagtgggaatgtgtgtgtgcgcacggcg | |
| | cagatgcattacaccatatgaattaacaccaggagccactgttcccttcctgctcagcctg | |
| | ctatgctgcgtcagaacgaccaaggcg | |
| 10 | ATYYEAAAYLWNEQQPLFWLQALIPLAALIVLCNCLKL | CHIKV (strain |
| | LPCCCKTLAFLAVMSIGAHTVSA | 37997) 6k protein- |
| | | amino acid |
| | | sequence |
| 11 | gccacatattacgaggctgcggcatatctatggaacgaacagcagcccctgttctggttg | CHIKV (strain |
| | caggctcttatcccgctggccgccttgatcgtcctgtgcaactgtctgaaactcttgccatg | 37997) 6k protein- |
| | ctgctgtaagaccctggcttttttagccgtaatgagcatcggtgcccacactgtgagcgc | nucleic acid |
| | g | sequence |
| 12 | YEHVTVIPNTVGVPYKTLVNRPGYSPMVLEMELQSVTL | CHIKV (strain |
| | EPTLSLDYITCEYKTVIPSPYVKCCGTAECKDKSLPDYS | 37997) Spike |
| | CKVFTGVYPFMWGGAYCFCDAENTQLSEAHVEKSESC | glycoprotein E1- |
| | KTEFASAYRAHTASASAKLRVLYQGNNITVAAYANGD | amino acid |
| | HAVTVKDAKFVVGPMSSAWTPFDNKIVVYKGDVYNM | sequence |
| | DYPPFGAGRPGQFGDIQSRTPESKDVYANTQLVLQRPA | |
| | AGTVHVPYSQAPSGFKYWLKERGASLQHTAPFGCQIAT | |
| | NPVRAVNCAVGNIPISIDIPDAAFTRVVDAPSVTDMSCE | |
| | VPACTHSSDFGGVAIIKYTASKKGKCAVHSMTNAVTIR | |
| | EADVEVEGNSQLQISFSTALASAEFRVQVCSTQVHCAA | |
| | ACHPPKDHIVNYPASHTTLGVQDISTTAMSWVQKITGG | |
| | VGLIVAVAALILIVVLCVSFSRH | |
| 13 | tacgaacacgtaacagtgatcccgaacacggtgggagtaccgtataagactcttgtcaa | CHIKV (strain |
| | cagaccgggttacagccccatggtgttggagatggagctacaatcagtcaccttggaac | 37997) Spike |
| | caacactgtcacttgactacatcacgtgcgagtacaaaactgtcatcccctccccgtacgt | glycoprotein E1- |
| | gaagtgctgtggtacagcagagtgcaaggacaagagcctaccagactacagctgcaa | nucleic acid |
| | ggtctttactggagtctacccatttatgtggggcggcgcctactgcttttgcgacgccgaa | sequence |
| | aatacgcaattgagcgaggcacatgtagagaaatctgaatcttgcaaaacagagtttgca | |
| | tcggcctacagagcccacaccgcatcggcgtcggcgaagctccgcgtcctttaccaag | |
| | gaaacaacattaccgtagctgcctacgctaacggtgaccatgccgtcacagtaaaggac | |
| | gccaagtttgtcgtgggcccaatgtcctccgcctggacaccttttgacaacaaaatcgtg | |
| | gtgtacaaaggcgacgtctacaacatggactacccaccttttggcgcaggaagaccag | |
| | gacaatttggtgacattcaaagtcgtacaccggaaagtaaagacgtttatgccaacactc | |
| | agttggtactacagaggccagcagcaggcacggtacatgtaccatactctcaggcacc | |
| | atctggcttcaagtattggctgaaggaacgaggagcatcgctacagcacacggcaccgt | |
| | tcggttgccagattgcgacaaacccggtaagagctgtaaattgcgctgtggggaacata | |
| | ccaatttccatcgacataccggatgcggcctttactagggttgtcgatgcaccctctgtaa | |
| | cggacatgtcatgcgaagtaccagcctgcactcactcctccgactttgggggcgtcgcc | |
| | atcatcaaatacacagctagcaagaaaggtaaatgtgcagtacattcgatgaccaacgc | |
| | cgttaccattcgagaagccgacgtagaagtagaggggaactcccagctgcaaatatcct | |
| | tctcaacagccctggcaagcgccgagtttcgcgtgcaagtgtgctccacacaagtacac | |
| | tgcgcagccgcatgccaccctccaaaggaccacatagtcaattacccagcatcacaca | |
| | ccacccttggggtccaggatatatccacaacggcaatgtcttgggtgcagaagattacg | |
| | ggaggagtaggattaattgttgctgttgctgccttaattttaattgtggtgctatgcgtgtcgt | |
| | ttagcaggcac | |

In some aspects, the VLP can comprise a CHIKV structural protein comprising a functional fragment of an amino acid sequence disclosed herein. In some aspects, the functional fragment is at least 85%, 90%, 95%, 96%, 97%, 98%, or 99% of the entire length of the CHIKV structural protein, and the CHIKV functional fragment can be used to form a recombinant VLP of the disclosure.

In some aspects, the VLP can comprise a CHIKV structural protein or fragment thereof comprising a sequence that is at least about 85%, 90%, 95%, 96%, 97%, 98%, 99%, or 100% identical to an amino acid sequence disclosed herein. In some aspects, a CHIKV Capsid (C) protein or functional fragment thereof comprises a sequence that is at least 85%, 90%, 95%, 96%, 97%, 98%, 99%, or 100% identical to the amino acid sequence of SEQ ID NO: 4, and can be used to form a recombinant VLP of the disclosure. In some aspects, a CHIKV Envelope 3 (E3) protein or functional fragment thereof comprises a sequence that is at least 85%, 90%, 95%, 96%, 97%, 98%, 99%, or 100% identical to the amino acid sequence of SEQ ID NO: 6, and can be used to form a recombinant VLP of the disclosure. In some aspects, a CHIKV Envelope 2 (E2) protein or functional fragment thereof comprises a sequence that is at least 85%, 90%, 95%, 96%, 97%, 98%, 99%, or 100% identical to the amino acid sequence of SEQ ID NO: 8, and can be used to form a recombinant VLP of the disclosure. In some aspects, a CHIKV 6K protein or functional fragment thereof comprises a sequence that is at least 85%, 90%, 95%, 96%, 97%, 98%, 99%, or 100% identical to the amino acid sequence of SEQ ID NO: 10, and can be used to form a recombinant VLP of the disclosure. In some aspects, a CHIKV Envelope 1 (E1) protein or functional fragment thereof comprises a sequence that is at least 85%, 90%, 95%, 96%, 97%, 98%, 99%, or 100% identical to the amino acid sequence of SEQ ID NO: 12, and can be used to form a recombinant VLP of the disclosure.

In some aspects, the VLP can comprise a CHIKV structural protein or fragment thereof encoded by a sequence that is at least about 85%, 90%, 95%, 98%, 99%, or 100% identical to a nucleic acid sequence disclosed herein. In some aspects, a CHIKV Capsid (C) protein or functional fragment thereof is encoded by a sequence that is at least 85%, 90%, 95%, 96%, 97%, 98%, 99%, or 100% identical to the nucleic acid sequence of SEQ ID NO: 5, and the encoded C protein can be used to form a recombinant VLP of the disclosure. In some aspects, a CHIKV Envelope 3 (E3) protein or functional fragment thereof is encoded by a sequence that is at least 85%, 90%, 95%, 96%, 97%, 98%, 99%, or 100% identical to the nucleic acid sequence of SEQ ID NO: 7, and the encoded E3 protein can be used to form a recombinant VLP of the disclosure. In some aspects, a CHIKV Envelope 2 (E2) protein or functional fragment thereof is encoded by a sequence that is at least 85%, 90%, 95%, 96%, 97%, 98%, 99%, or 100% identical to the nucleic acid sequence of SEQ ID NO: 9, and the encoded E2 protein can be used to form a recombinant VLP of the disclosure. In some aspects, a CHIKV 6K protein or functional fragment thereof is encoded by a sequence that is at least 85%, 90%, 95%, 96%, 97%, 98%, 99%, or 100% identical to the nucleic acid sequence of SEQ ID NO: 11, and the encoded 6K protein can be used to form a recombinant VLP of the disclosure. In some aspects, a CHIKV Envelope 1 (E1) protein or functional fragment thereof is encoded by a sequence that is at least 85%, 90%, 95%, 96%, 97%, 98%, 99%, or 100% identical to the nucleic acid sequence of SEQ ID NO: 13, and the encoded E1 protein can be used to form a recombinant VLP of the disclosure.

Certain aspects of the disclosure include VLPs comprising one or more CHIKV polypeptides. Also included are VLPs comprising one or more CHIKV polypeptides or fragments thereof that are modified in ways that enhance or do not inhibit their ability to modulate an immune response. In some aspects, the CHIKV amino acid sequence or nucleic acid sequence comprises an alteration. Such alterations can include certain mutations, deletions, insertions, or post-translational modifications. In some aspects, analogs of naturally-occurring polypeptide of the disclosure are included. Analogs can differ from the naturally-occurring the polypeptide by amino acid sequence differences, by post-translational modifications, or by both. Analogs of the disclosure can exhibit at least 85%, 90%, 95%, 96%, 97%, 98%, or 99% identity with all or part of a naturally-occurring amino acid sequence. The length of sequence comparison can be at least 10, 13, 15 amino acid residues, at least 25 amino acid residues, or more than 35 amino acid residues.

Alterations of a CHIKV polypeptide can include, but are not limited to, site-directed, random point mutagenesis, homologous recombination (DNA shuffling), mutagenesis using uracil containing templates, oligonucleotide-directed mutagenesis, phosphorothioate-modified DNA mutagenesis, mutagenesis using gapped duplex DNA or the like. Additional suitable methods include point mismatch repair, mutagenesis using repair-deficient host strains, restriction-selection and restriction-purification, deletion mutagenesis, mutagenesis by total gene synthesis, double-strand break repair, and the like. Mutagenesis, e.g., involving chimeric constructs, is also included in the present disclosure. In certain aspects, mutagenesis can be guided by known information of the naturally occurring molecule or altered or mutated naturally occurring molecule, e.g., sequence, sequence comparisons, physical properties, crystal structure or the like.

In certain aspects, the disclosure provides polypeptide variants that differ from a reference polypeptide. The term “variant” refers to an amino acid sequence that is altered by one or more amino acids with respect to a reference sequence. The variant can have “conservative” changes, wherein a substituted amino acid has similar structural or chemical properties, e.g., replacement of leucine with isoleucine. Alternatively, a variant can have “nonconservative” changes, e.g., replacement of a glycine with a tryptophan. Analogous minor variations can also include amino acid deletion or insertion, or both. Guidance in determining which amino acid residues can be substituted, inserted, or deleted without eliminating biological or immunological activity can be found using computer programs well known in the art, for example, DNASTAR software. Desirably, variants show substantial biological activity. In some aspects, a protein variant forms a VLP and elicits an antibody response when administered to a subject.

In addition to full-length polypeptides, the disclosure also includes functional fragments of the polypeptides of the disclosure.

### Immunogenic Compositions and Vaccines

Certain aspects of the disclosure are directed to a composition or vaccine which comprises a protein-based non-infectious CHIKV virus-like particle (VLP) disclosed herein. In certain aspects, the composition or vaccine disclosed herein can comprise: a virus-like particle (VLP) which comprises a capsid protein and an envelope protein derived from Chikungunya virus (CHIKV), wherein the composition or vaccine induces a neutralizing antibody response against CHIKV in a subject within 7 days after administration of a single dose of the composition or vaccine to the subject and/or induces a neutralizing antibody response against CHIKV which persists for at least 28 days, at least 6 months, at least 1 year, or at least 2 years after a single dose is administered to the subject. In some aspects, the serum neutralizing response comprises a serum neutralizing antibody titer of ≥20, ≥25, ≥30, ≥35, ≥40, ≥45, or ≥50 (e.g., ≥40). In some aspects, the composition or vaccine further comprises an adjuvant (e.g., an aluminum hydroxide adjuvant).

In certain aspects, the composition or vaccine of the disclosure comprises CHIKV VLPs which comprise one or more CHIKV VLP proteins. In certain aspects, a VLP of the composition or vaccine comprises at least three recombinant CHIKV structural proteins, e.g., a capsid core protein (C), an Envelope 1 protein (E1), and an Envelope 2 protein (E2). In some aspects, the VLP can further comprise an Envelope 3 protein (E3) and/or a 6k protein (6K). In some aspects, the capsid protein is a CHIKV C protein. In some aspects, the envelope protein is a CHIKV protein selected from the group consisting of E3, E2, 6K, E1, and any combination thereof. In some aspects, the CHIKV envelope proteins E2 and E1. Some aspects the VLP of the composition or vaccine comprises CHIKV C, E2, and E1 proteins. In certain aspects, the VLP of the composition or vaccine does not comprise CHIKV E3 and/or 6K. In some aspects, the composition or vaccine capsid and envelope proteins are derived from CHIKV strain 37997. In certain aspects, the composition or vaccine comprises CHIKV VLPs which comprise Capsid, E1, and E2 proteins in an amount of at least 95%, at least 96%, or at least 97% of the total CHIKV proteins present in the composition or vaccine.

In certain aspects, the composition or vaccine of the disclosure comprises VLP in an amount of about 6 μg to 200 μg, about 10 μg to 200 μg, about 20 μg to 200 μg, about 20 μg to 150 μg, about 20 μg to 100 μg, about 20 μg to 80 μg, about 20 μg to 60 μg, or about 20 μg to 50 μg, e.g., in a single dose formulation (e.g., in a volume of between about 0.5 mL to 1.0 mL). In some aspects, the single dose of the composition or vaccine comprises VLP in an amount of about 6 μg to 60 μg (e.g., about 40 μg VLP). In some aspects, the composition or vaccine comprises about 6 μg to 60 μg of VLP. In some aspects, the composition or vaccine comprises about 6 μg, about 10 μg, about 20 μg, about 22 μg, about 24 μg, about 26 μg, about 28 μg, about 30 μg, about 32 μg, about 34 μg, about 36 μg, about 38 μg, about 40 μg, about 42 μg, about 44 μg, about 46 μg, about 48 μg, about 50 μg, or about 60 μg of VLP. In some aspects, the composition or vaccine comprises about 40 μg of VLP. In certain aspects, the composition or vaccine of the disclosure comprises VLP in a dosage amount of about 7.5 μg/mL to 75 μg/mL, about 12.5 μg/mL to 75 μg/mL, 25 μg/mL to 75 μg/mL, 37.5 μg/mL to 62.5 μg/mL, or 40 μg/mL to 60 μg/mL. In certain embodiments, the composition or vaccine of the disclosure comprises VLP in a dosage amount of about 25 μg/mL, about 50 μg/mL, or about 75 μg/mL. In certain embodiments, the composition or vaccine of the disclosure comprises VLP in a dosage amount of about 50 μg/mL. In some aspects, the VLP of the disclosure can be adjuvanted with an aluminum adjuvant, e.g., aluminum hydroxide gel (e.g., Alhydrogel®). In some aspects, the composition or vaccine comprises about 40 μg per about 300 μg of aluminum hydroxide gel (e.g., Alhydrogel®).

In certain aspects, the adjuvant is aluminum hydroxide gel (e.g., Alhydrogel®) (also referred to as alum). In some aspects, the composition or vaccine can comprise aluminum hydroxide gel (e.g., Alhydrogel®) in an amount of about 30 μg to 3000 μg, about 50 μg to 2500 μg, about 100 μg to 2000 μg, about 100 μg to 1000 μg, about 100 μg to 750 μg, about 100 μg to 500 μg, about 150 μg to 500 μg, or about 200 μg to 400 μg, e.g., in a single dose formulation (e.g., in a volume of between about 0.5 mL to 1.0 mL). In some aspects, the single dose of the composition or vaccine comprises aluminum hydroxide gel (e.g., Alhydrogel®) in an amount of about 100 μg to 500 μg (e.g., about 300 μg aluminum hydroxide gel (e.g., Alhydrogel®)). In some aspects, the single dose comprises about 250 μg/mL to 625 μg/mL aluminum hydroxide gel (e.g., Alhydrogel®). In some aspects, the single dose comprises about 375 μg/mL aluminum hydroxide gel (e.g., Alhydrogel®). In some aspects, the composition or vaccine can comprise about 300 μg aluminum hydroxide gel (e.g., Alhydrogel®) per about 40 μg of the VLP.

In certain aspects, the composition or vaccine of the disclosure comprises a sugar. In some aspects, the sugar is selected from the group consisting of sucrose, lactose, and any combination thereof. In some aspects, the sugar is sucrose. In some aspects, the composition or vaccine can comprise a sugar (e.g., sucrose) in an amount of about 10 mg to 200 mg, about 10 mg to 150 mg, about 10 mg to 100 mg, about 20 mg to 200 mg, about 20 mg to 150 mg, about 20 mg to 100 mg, about 30 mg to 100 mg, about 40 mg to 100 mg, or about 40 mg to 80 mg, e.g., in a single dose formulation (e.g., in a volume of between about 0.5 mL to 1.0 mL). In some aspects, the single dose of the composition or vaccine comprises a sugar (e.g., sucrose) in an amount of about 40 mg to 80 mg (e.g., 50 mg to 70 mg sugar). In some aspects, the composition or vaccine comprises about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, or about 64 μg of VLP. In some aspects, the composition or vaccine comprises about 59 mg to 60 mg (e.g., 59.7 mg) of VLP.

In certain aspects, the composition or vaccine further comprises a buffer and/or pH modifier. In some aspects, the buffer and/or pH modifier is selected from the group consisting of potassium phosphate monobasic, potassium phosphate dibasic, sodium citrate dehydrate, and any combination thereof. In some aspects, the composition or vaccine can comprise a buffer and/or pH modifier in an amount of about 0.2 mg to 20 mg, about 0.2 mg to 15 mg, about 0.2 mg to 10 mg, or about 0.4 mg to 10 mg, e.g., in a single dose formulation (e.g., in a volume of between about 0.5 mL to 1.0 mL). In some aspects, the single dose of the composition or vaccine comprises a buffer and/or pH modifier (e.g., one or more of potassium phosphate monobasic, potassium phosphate dibasic, or sodium citrate dehydrate) in an amount of about 0.2 mg to 20 mg (e.g., 0.2 mg to 10 mg buffer and/or pH modifier). In certain aspects, the composition or vaccine comprises about 0.2 mg to 1.0 mg of potassium phosphate monobasic (e.g., about 0.4 mg potassium phosphate monobasic); about 0.2 mg to 1.5 mg of potassium phosphate diabasic (e.g., about 0.9 mg potassium phosphate diabasic); and/or about 2 mg to 10 mg sodium citrate dehydrate (e.g., about 5.9 mg sodium citrate dehydrate).

In certain aspects, the composition or vaccine further comprises a carrier, a diluent or other excipient, e.g., a pharmaceutically acceptable carrier. Pharmaceutically acceptable carriers can include, but are not limited to water, saline, buffered saline, glycerol, sterile isotonic aqueous buffer, and combinations thereof. A thorough discussion of pharmaceutically acceptable carriers, diluents, and other excipients is presented in Remington's Pharmaceutical Sciences (Mack Pub. Co. N.J. current edition). In some aspects, the carrier is water.

In certain aspects, the composition or vaccine does not comprise a preservative or antibiotic.

In certain aspects, the composition or vaccine disclosed herein can comprise: (a) a virus-like particle (VLP) which comprises a capsid protein and an envelope protein derived from Chikungunya virus (CHIKV); and (b) an adjuvant (e.g., an aluminum hydroxide adjuvant). In certain aspects, the composition or vaccine comprises: (a) about 6 μg to 60 μg (e.g., 40 μg) of the VLP; and (b) about 200 μg to 500 μg (e.g., 300 μg) of aluminum hydroxide in a volume between about 0.5 mL to 1.0 mL (e.g., about 0.8 mL).

In some aspects, the composition or vaccine comprises: (a) about 6 μg to 60 μg (e.g., about 40 μg) of the VLP; (b) about 200 μg to 500 μg of the aluminum hydroxide (e.g., about 300 μg); (c) about 20 mg to 80 mg of sucrose (e.g., about 55 mg to 65 mg); (d) about 0.2 mg to 1.0 mg of potassium phosphate monobasic (e.g., about 0.2 mg to 0.8 mg); (e) about 0.2 mg to 1.5 mg of potassium phosphate diabasic (e.g., about 0.5 to 1.5 mg); (f) about 2 mg to 10 mg sodium citrate dehydrate (e.g., about 5.5 mg to 6.5 mg); and (g) water in a volume between about 0.5 mL to 1.0 mL (e.g., about 0.8 mL).

In some aspects, the composition or vaccine is formulated as a suspension, e.g., for intramuscular injection. The composition or vaccine of the disclosure can be a suspension suitable for a single dose intramuscular injection (e.g., a 0.8 mL dose in a pre-filled syringe). In some aspects, the composition or vaccine is formulated as a suspension and stored in a vial at 2 to 8° C. (36 to 46° F.) prior to use. In some aspects, the composition or vaccine is formulated as a suspension and stored in a pre-filled syringe at 2 to 8° C. (36 to 46° F.) prior to use.

In certain aspects, the composition or vaccine of the disclosure is administered in an effective amount. In some aspects, the effective amount is an amount of the composition or vaccine of the disclosure that provides a neutralizing antibody response and/or protection against CHIKV within 7 days.

In certain aspects, the composition or vaccine disclosed herein induces a neutralizing antibody response against CHIKV within 7 days of administration, e.g., a single dose administration. In some aspects, the composition or vaccine induces antibodies against homologous or heterologous strains of CHIKV. In some aspects, the neutralizing antibody response against CHIKV is sustained for at least 21 days, at least 22 days, at least 28 days, at least three months, at least 4 months, at least 5 months, at least 6 months, at least 7 months, at least 8 months, at least 9 months, at least 10 months, at least 11 months, at least one year, at least 18 months, or at least two years after administration, e.g., of a single dose administration. In some aspects, the neutralizing antibody response against CHIKV is sustained for the lifetime of the subject after administration, e.g., of a single dose administration or after a single dose administration and one or more booster dose administration(s).

In certain aspects, the composition or vaccine disclosed herein induces a serum neutralizing antibody titer of ≥20, ≥25, ≥30, ≥35, ≥40, ≥45, or ≥50 (e.g., ≥40) within 7 days after administration, e.g., measured in human sera by a CHIKV neutralization assay (e.g., NT80 Serum Neutralizing Antibodies by Luciferase Assay (SNA) or plaque assay). In certain aspects, the composition or vaccine disclosed herein induces a serum neutralizing antibody titer of between 20 to 10,000, 20 to 15,000, 30 to 10,000, 30 to 15,000, 40 to 10,000, or 40 to 15,000 within 7 days after administration, e.g., measured in human sera by a CHIKV neutralization assay (e.g., NT80 Serum Neutralizing Antibodies by Luciferase Assay (SNA) or plaque assay). In some aspects, the serum neutralizing antibody titer≥20, ≥25, ≥30, ≥35, ≥40, ≥45, or ≥50 (e.g., ≥40) is sustained for at least 21 days, at least 22 days, at least 28 days, at least 3 months, at least 6 months, at least 12 months, at least 18 months, and/or at least 2 years. In some aspects, the serum neutralizing antibody titer≥20, ≥25, ≥30, ≥35, ≥40, ≥45, or ≥50 (e.g., ≥40) is sustained for at least 30 months, 2 years, 3 years, 4 years, 5 years, 10 years, or 20 years or more. In some aspects, the serum neutralizing antibody titer≥20, ≥25, ≥30, ≥35, ≥40, ≥45, or ≥50 (e.g., ≥40) is sustained for the lifetime of the subject after administration of, e.g., a single dose administration or a single dose administration and one or more booster dose administration(s). In certain aspects, at least 60%, at least 70%, at least 80%, at least 90%, at least 95%, at least 98%, at least 99%, or more of the subjects vaccinated with a composition or vaccine disclosed herein produce a neutralizing antibody response to CHIKV 7 days after administration of the VLP. In certain aspects, at least 60%, at least 70%, at least 80%, at least 90%, or at least 95% of the population sustains a serum neutralizing antibody titer≥20, ≥25, ≥30, ≥35, ≥40, ≥45, or ≥50 (e.g., ≥40) for at least 21 days, at least 22 days, at least 28 days, at least 3 months, at least 6 months, at least 12 months, at least 18 months, and/or at least 2 years after administration of a composition or vaccine disclosed herein (e.g., after a single dose or after a single dose administration and one or more booster dose administration(s)). In some aspects a second dose, e.g., a booster dose, of a composition or vaccine disclosed herein is administered at least 6 months, at least 9 months, at least 12 months, at least 14 months, at least 16 months, at least 17 months, at least 18 months, at least 20 months, at least 22 months, or at least 2 years after a first single dose of a composition or vaccine disclose herein. In some aspects, one or more booster dose(s) can be administered throughout the lifetime of the subject to maintain a protective serum neutralizing antibody titer≥20, ≥25, ≥30, ≥35, ≥40, ≥45, or ≥50 (e.g., ≥40).

In certain aspects, the neutralizing antibody response develops within 7 days, with a seroresponse rate of at least 60%, at least 70%, or at least 80% within 7 days after vaccination. In certain aspects, the response persists for at least 2 years after vaccination, with a seroresponse rate of at least 50% or at least 60%.

### Dosing and Administration

Certain aspects of the disclosure are directed to dosing and administration of a composition or vaccine disclosed herein. The compositions and vaccines disclosed herein can be administered as either a single dose (e.g., one time dose) or multiple doses (e.g., prime and booster doses). In some aspects, the composition or vaccine is formulated as a single dose. In some embodiments, the single dose of the composition or vaccine is administered once. In some aspects, the single dose of the composition or vaccine is administered as one or more booster doses (e.g., at least 2, at least 3, at least 4, at least 5, or at least 6 doses). In certain aspects, a first dose (e.g., a prime dose) and any subsequent booster dose can be the same or different (e.g., more or less VLP per dose). In some aspects, the single dose of the composition or vaccine is administered once and a booster dose is administered at least 6 months, at least 9 months, at least 12 months, at least 14 months, at least 16 months, at least 17 months, at least 18 months, at least 20 months, at least 22 months, or at least 2 years after a first single dose of a composition or vaccine disclosed herein. In some aspects, one or more booster dose(s) can be administered throughout the lifetime of the subject to maintain a protective serum neutralizing antibody titer.

In some aspects, the composition or vaccine of the disclosure is formulated for intramuscular (IM) or intravenous (IV) administration. In certain aspects the administration is intramuscular (IM). In some aspects, a single dose of a composition or vaccine disclosed herein is administered intramuscularly to the upper arm.

In certain aspects, the composition or vaccine of the disclosure is formulated and/or administered as a single dose for one-time administration. This aspect of the disclosure is in contrast to commercial HPV, HEV, and HAV VLP-based vaccines including Aluminum salt-based adjuvants, which are each administered as multiple doses (See, e.g., Cimica and Galaza, Clin Immunol. 2017; 183: 99-108).

In certain aspects, a single dose (e.g., a one-time single dose, a prime dose and/or a booster dose) of a VLP of the disclosure comprises about 6 μg to 200 μg, about 6 μg to 100 μg, or about 6 μg to 60 μg (e.g., about 20 μg to 60 μg, e.g., about 40 μg), for example formulated in a pre-filled syringe, e.g., for a single dose volume of about 0.5 mL to 1.0 mL (e.g., about 0.8 mL). In certain aspects, a single dose (e.g., a one-time single dose, a prime dose and/or a booster dose) of the composition or vaccine of the disclosure comprises VLP in a dosage amount of about 7.5 μg/mL to 75 μg/mL, about 12.5 μg/mL to 75 μg/mL, 25 μg/mL to 75 μg/mL, 37.5 μg/mL to 62.5 μg/mL, or 40 μg/mL to 60 μg/mL. In certain embodiments, a single dose (e.g., a one-time single dose, a prime dose and/or a booster dose) of the composition or vaccine of the disclosure comprises VLP in a dosage amount of about 25 μg/mL, about 50 μg/mL, or about 75 μg/mL. In certain embodiments, a single dose (e.g., a one-time single dose, a prime dose and/or a booster dose) of the composition or vaccine of the disclosure comprises VLP in a dosage amount of about 50 μg/mL. In some aspects, the VLP is adjuvanted with an aluminum adjuvant, e.g., aluminum hydroxide gel (e.g., Alhydrogel®). In some aspects, the aluminum hydroxide gel (e.g., Alhydrogel®) is in an amount of about 100 μg to 500 μg (e.g., about 300 μg aluminum hydroxide gel (e.g., Alhydrogel®)). In some aspects, a single dose (e.g., a one-time single dose, a prime dose and/or a booster dose) of the composition or vaccine of the disclosure comprises about 250 μg/mL to 625 μg/mL aluminum hydroxide gel (e.g., Alhydrogel®). In some aspects, the single dose comprises about 375 μg/mL aluminum hydroxide gel (e.g., Alhydrogel®). In some aspects, the dosage form is a suspension, e.g., for intramuscular administration.

In certain aspects, a single dose (e.g., a one-time single dose, a prime dose and/or a booster dose) of a composition or vaccine disclosed herein comprises a volume of about 0.5 mL to 5 mL, about 0.5 mL to 4 mL, about 0.5 mL to 4 mL, about 0.5 mL to 2 mL, or about 0.5 mL to 1 mL. In some aspects, a single dose of a composition or vaccine disclosed herein comprises a volume of about 0.5 mL, about 0.6 mL, about 0.7 mL, about 0.8 mL, about 0.9 mL, or about 1.0 mL. In some aspects, the single dose of the composition or vaccine comprises VLP in an amount of about 6 μg to 60 μg (e.g., about 20 μg to 60 VLP). In some aspects, the composition or vaccine comprises about 40 μg of VLP. In some aspects, the VLP of the disclosure can be adjuvanted with an aluminum adjuvant, e.g., aluminum hydroxide gel (e.g., Alhydrogel®). In some aspects, the aluminum hydroxide gel (e.g., Alhydrogel®) is in an amount of about 100 μg to 500 μg (e.g., about 300 μg aluminum hydroxide gel (e.g., Alhydrogel®)). In some aspects, a single dose of the composition or vaccine of the disclosure comprises about 40 μg per about 300 μg of aluminum hydroxide gel (e.g., Alhydrogel®) formulated for a single administration dose comprising about 0.5 mL to 1.0 mL (e.g., about 0.8 mL).

In certain aspects, each dose of the composition or vaccine disclosed herein can comprise about 6 μg to about 60 μg (e.g., about 40 μg) of CHIKV VLP, about 100 μg to 500 μg (e.g., about 300 μg) of aluminum (e.g., as aluminum hydroxide adjuvant), about 20 mg to 80 mg (e.g., about 59.7 mg) of sucrose, about 0.2 mg to 0.8 mg (e.g., about 0.4 mg) of potassium phosphate monobasic, about 0.5 mg to 1.5 mg (e.g., about 0.9 mg) of potassium phosphate dibasic, about 1 mg to 10 mg (e.g., about 5.9 mg) of sodium citrate dihydrate, and water for injection.

In certain aspects, the composition or vaccine of the disclosure is administered in an effective amount. In some aspects, the effective amount is an amount of the composition or vaccine of the disclosure that provides a neutralizing antibody response and/or protection against CHIKV within 7 days. In some embodiments, an effective amount is achieved with a single one time dose. In some embodiments, an effective amount is achieved with a single dose and one or more booster doses.

The methods disclosed herein also include a one-time single dose as well as a variety of prime-boost regimens. In certain aspects, one or more priming immunizations can be followed by one or more boosting immunizations. The immunogenic composition or vaccine can be the same or different for each immunization, and the dosage amount of the immunogenic composition or vaccine, the route, and formulation can also be varied.

### Methods of Use

Certain aspects of the disclosure are directed to use of a composition, a vaccine, and/or a dosage form disclosed herein to provide active immunization to prevent or reduce the risk of disease caused by Chikungunya virus (CHIKV) infection in an individual or a population. In some aspects, the subject is an adult (e.g., ≥12 years old) or a child (e.g., ≤12 years old, 6 months to ≤12 years old, 1 year to ≤12 years old).

Certain aspects of the disclosure provide compositions and methods for inducing an immunological response in a subject, particularly a human, which comprises administering to the subject a VLP comprising CHIKV polypeptides, or fragments thereof, formulated for inducing or enhancing an immune response. In some aspects, an immune response protects the subject from a CHIKV infection, or inflammatory symptoms thereof (e.g., arthritis). The administration of an immunological composition or vaccine disclosed herein can be used either therapeutically in subjects already experiencing a CHIKV infection, or can be used prophylactically to prevent or reduce the risk of a CHIKV infection.

In certain aspects, the methods comprise administering a composition, vaccine, or dosage form disclosed herein in an effective amount. In some aspects, the effective amount is an amount of the composition or vaccine of the disclosure that provides a neutralizing antibody response and/or protection against CHIKV within 7 days. In some embodiments, an effective amount is achieved with administration of a single one-time dose. In some embodiments, an effective amount is achieved with administration of a single dose and one or more booster doses. In some aspects, the neutralizing antibody response against CHIKV persists for at least 28 days, at least 6 months, at least 1 year, or at least 2 years after administration of a single dose or a single dose and one or more booster dose(s). In some aspects, the neutralizing antibody response against CHIKV persists for at least 30 months, at least 2 years, at least 3 years, at least 4 years, at least 5 years, at least 10 years, or 20 years or more after administration of a single dose or a single dose and one or more booster dose(s). In some aspects, the neutralizing antibody response against CHIKV persists for the lifetime of the subject after administration of a single dose or a single dose and one or more booster dose(s). In some aspects, the serum neutralizing response comprises a serum neutralizing antibody titer of ≥20, ≥25, ≥30, ≥35, ≥40, ≥45, or ≥50 (e.g., ≥40).

In some aspects, the disclosure is directed to a method of inducing a neutralizing antibody response to CHIKV in a subject, comprising administering to the subject a composition, a vaccine, and/or a dosage form disclosed herein. In some aspects, the neutralizing antibody response is against the E1 and/or E2 proteins of CHIKV.

Certain aspects of the disclosure are directed to methods of eliciting an immune response and/or protective immunity against CHIKV in a subject, comprising administering to the subject a composition, a vaccine, and/or a dosage form disclosed herein. In certain aspects, the immunity protects against at least one, more than one or all lineages of CHIKV (e.g., one or more of ECSA, Asian, and/or West African).

In some aspects, the disclosure directed to a method of treating, preventing, or reducing the risk of a Chikungunya infection in a subject, comprising administering to a subject in need thereof a composition, a vaccine, and/or a dosage form disclosed herein. In some aspects, the protection against CHIKV disease is provided by eliciting neutralizing antibodies against E1 and/or E2 proteins of CHIKV.

Certain aspects of the disclosure are directed to a method of eliciting an immune response and/or protective immunity against Chikungunya virus (CHIKV) in a subject comprising: administering to the subject a composition, a vaccine, and/or a dosage form comprising (a) virus-like particles (VLPs) which comprise a capsid protein and an envelope protein derived from CHIKV, wherein a single dose of the composition, vaccine, and/or dosage form comprises about 6 μg to 60 μg VLPs (e.g., about 40 μg), and wherein the administration of the single dose of the composition, vaccine, and/or dosage form elicits production of CHIKV-specific neutralizing antibodies in the subject within 7 days of administration and/or elicits production of CHIKV-specific neutralizing antibodies for at least 28 days, at least 6 months, at least 1 year, or at least 2 years after a single dose is administered to the subject. In some aspects, the CHIKV-specific neutralizing antibody titer comprises ≥20, ≥25, ≥30, ≥35, ≥40, ≥45, or ≥50 (e.g., ≥40). In certain aspects, the composition, vaccine, and/or dosage form further comprises an adjuvant (e.g., an aluminum adjuvant). In some aspects, the CHIKV-specific neutralizing antibodies are against E1 and/or E2 proteins of CHIKV.

In some aspects, the disclosure is directed to vaccinating a subject or population against a CHIKV infection, comprising administering to the subject or the population a composition, a vaccine, and/or a dosage form disclosed herein.

In certain aspects, the composition, vaccine, and/or dosage form disclosed herein induces a neutralizing antibody response against CHIKV within 7 days of administration, e.g., a single dose administration. In some aspects, the composition, vaccine, and/or dosage form induces antibodies against homologous or heterologous strains of CHIKV. In some aspects, the neutralizing antibody response against CHIKV is sustained for at least 21 days, at least 22 days, at least 28 days, at least three months, at least 4 months, at least 5 months, at least 6 months, at least 7 months, at least 8 months, at least 9 months, at least 10 months, at least 11 months, at least on year after administration, at least 18 months, or at least two years, e.g., a single dose administration. In some aspects, the neutralizing antibody response and/or protective immunity against CHIKV is sustained for at least 1 year, at least 18 months, at least 30 months, at least 2 years, at least 3 years, at least 4 years, at least 5 years, at least 10 years, or 20 years or more after administration of a single dose or a single dose and one or more booster dose(s). In some aspects, the neutralizing antibody response and/or protective immunity against CHIKV is sustained for the lifetime of the subject after administration of a single dose or a single dose and one or more booster dose(s).

In certain aspects, the composition, vaccine, and/or dosage form disclosed herein induces a serum neutralizing antibody titer≥20, ≥25, ≥30, ≥35, ≥40, ≥45, or ≥50 (e.g., ≥40) within 7 days after administration, e.g., measured in human sera by a CHIKV neutralization assay (e.g., NT80 Serum Neutralizing Antibodies by Luciferase Assay (SNA)). In some aspects, the serum neutralizing antibody titer≥40 is sustained for at least 21 days, at least 22 days, at least 28 days, at least 3 months, at least 6 months, at least 12 months, at least 18 months, and/or at least 2 years.

In certain aspects, at least 60%, at least 70%, at least 80%, at least 90%, at least 95%, at least 98%, at least 99%, or more of the subjects vaccinated with a composition, a vaccine, and/or a dosage form disclosed herein produce a neutralizing antibody response to CHIKV 7 days after administration of the VLP. In certain aspects, at least 60%, at least 70%, at least 80%, at least 90%, or at least 95% of a population vaccinated with a composition, a vaccine, and/or a dosage form disclosed herein sustains a serum neutralizing antibody titer≥20, ≥25, ≥30, ≥35, ≥40, ≥45, or ≥50 (e.g., ≥40) for at least 21 days, at least 22 days, at least 28 days, at least 3 months, at least 6 months, at least 12 months, at least 18 months, and/or at least 2 years after administration a composition, a vaccine, and/or a dosage form disclosed herein (e.g., after a single dose). In some aspects a single dose of a composition, a vaccine, and/or a dosage form disclosed herein sustains a serum neutralizing antibody titer≥20, ≥25, ≥30, ≥35, ≥40, ≥45, or ≥50 (e.g., ≥40) for at least 1 year, at least 18 months, at least 30 months, at least 2 years, at least 3 years, at least 4 years, at least 5 years, at least 10 years, 15 years, or 20 years or more after a single dose. In some aspects a second dose, e.g., a booster dose, of a composition, a vaccine, and/or a dosage form disclosed herein is administered at least 6 months, at least 9 months, at least 12 months, at least 14 months, at least 16 months, at least 17 months, at least 18 months, at least 20 months, at least 22 months, or at least 2 years after a first single dose of a composition, a vaccine, and/or a dosage form disclose herein. In some aspects a second dose, e.g., a booster dose, of a composition, a vaccine, and/or a dosage form disclosed herein is administered at least 1 year, at least 18 months, at least 30 months, at least 2 years, at least 3 years, at least 4 years, at least 5 years, at least 10 years, at least 15 years, or 20 years or more after a first single dose of a composition, a vaccine, and/or a dosage form disclose herein.

In certain aspects, at least 60%, at least 70%, at least 80%, at least 90%, at least 95%, at least 96%, at least 97%, at least 98%, at least 99%, or more of the subjects vaccinated with a composition, a vaccine, and/or a dosage form disclosed herein has seroconversion by 7 day after administration of the VLP-containing composition, vaccine, and/or dosage form (e.g., one single dose). In certain aspects, 60% to 99%, 60% to 98%, 65% to 98%, 70% to 98%, or 74% to 98% of the subjects vaccinated with a composition, a vaccine, and/or a dosage form disclosed herein has seroconversion by 7 day after administration of the VLP-containing composition, vaccine, and/or dosage form (e.g., one single dose). In certain aspects, at least 90%, at least 95%, at least 96%, at least 97%, at least 98%, at least 99%, or more of the subjects vaccinated with a composition, a vaccine, and/or a dosage form disclosed herein has seroconversion by Day 25, Day 26, Day 27, Day 28 day, Day 29, or Day 30 after administration of the VLP-containing composition, vaccine, and/or dosage form (e.g., a one-time single dose). In certain aspects, the immune response is persistent for at least 6 months, at least 9 months, at least 12 months, at least 18 months, or at least 24 months.

In certain aspects, the neutralizing antibody response vaccinates the subject against multiple serotypes, genotypes or lineages of CHIKV (ECSA (e.g., Tanzania 1953, India 2000, Malaysia 2008), Asian (e.g., St. Martin 2013, Indonesia 2007), and West African (e.g., Nigeria 1965, Senegal 2005, Senegal 1983)). In some aspects, the neutralizing antibody response is ≥75% IgG3, ≥80% IgG3, ≥85% IgG3, or ≥90% IgG3.

In certain aspects, the neutralizing antibody response against CHIKV is sustained for at least 28 days, 6 months, one year, 18 months, or two years after a single dose is administered. In certain aspects, a second (booster) dose of the composition or vaccine is administered to the subject about 28 days, about 3 months, about 6 months, about one year, about 18 months, or about 2 years after an earlier single dose. In some aspects, the neutralizing antibody response provided by a single dose is sustained for at least 1 year. In some aspects, a second dose (booster dose) is not administered within 9 months, 12 months, 18 months, 2 years, 2.5 years, 3 years, 3.5 years, 4 years, 4.5 years, 5 years, or 5.5 years from receiving the initial dose.

In certain aspects, the neutralizing antibody response against CHIKV is sustained for at least about six months, at least about 1 year, at least about 18 months, or at least about two years after administration of the second dose.

In certain aspects, an individual develops a serum neutralizing antibody titer≥20, ≥25, ≥30, ≥35, ≥40, ≥45, or ≥50 (e.g., ≥40) within 7 days after administration of a single dose of a composition, a vaccine, and/or a dosage form disclosed herein. In certain aspects, an individual maintains a serum neutralizing antibody titer≥20, ≥25, ≥30, ≥35, ≥40, ≥45, or ≥50 (e.g., ≥40) for at least about one year or about two years after administration of a single dose of the composition, vaccine, and/or dosage form. In certain aspects, an individual maintains a serum neutralizing antibody titer≥20, ≥25, ≥30, ≥35, ≥40, ≥45, or ≥50 (e.g., ≥40) for the lifetime of the subject after administration of a single dose of the composition, vaccine, and/or dosage form.

In certain aspects, at least 60%, at least 70%, at least 80%, at least 90%, at least 95%, at least 96%, at least 97%, or at least 98% of a vaccinated population develops a serum neutralizing antibody titer≥20, ≥25, ≥30, ≥35, ≥40, ≥45, or ≥50 (e.g., ≥40) within 7 days after administration of a single dose of a composition, a vaccine, and/or a dosage form disclosed herein.

In certain aspects, at least 60%, at least 70%, at least 80%, at least 90%, at least 95%, at least 96%, at least 97%, or at least 98% of a vaccinated population maintains a serum neutralizing antibody titer≥20, ≥25, ≥30, ≥35, ≥40, ≥45, or ≥50 (e.g., ≥40) for 1 year or two years after administration of a single dose of the composition, vaccine, and/or dosage form.

In certain aspects, the seroresponse rate is at least 60%, at least 70%, at least 80%, at least 90%, at least 95%, at least 96%, at least 97%, or at least 98% within 7 days, 28 days, 6 months, 9 months, one year, 18 months, or two years after administration of the composition, vaccine, and/or dosage form disclosed herein.

In some aspects, the composition, vaccine, and/or dosage form disclosed herein can be administered as part of a combination therapy. For example, the composition, vaccine, and/or dosage form disclosed herein can be co-administered or formulated with other immunogenic compositions, antivirals and/or antibiotics. A composition, vaccine, and/or dosage form disclosed herein can be administered concurrently, subsequent to, or sequentially with another immunogenic composition, antiviral, antibiotic, or any other agent that prevents or treats a Chikungunya infection.

### CHIKV VLP Vaccine Production

Certain aspects of the disclosure are directed to preparing and formulating VLPs disclosed herein as immunogenic compositions or vaccines.

Recombinant constructs can be prepared and used to transfect, infect, or transform and can express viral proteins, including those described herein, into eukaryotic cells and/or prokaryotic cells. Thus, the disclosure provides for host cells which comprise a vector (or vectors) that contain nucleic acids which code for CHIKV structural genes, including capsid, E3, E2, 6K, and E1 or portions thereof, and/or any chimeric molecule described above, and permit the expression of CHIKV structural genes, including capsid E3, E2, 6K, and E1, or portions thereof, and/or any chimeric molecule described herein in said host cell under conditions which allow the formation of VLPs.

In certain aspects, non-infectious recombinant VLPs comprising one or more CHIKV polypeptides (e.g., CHIKV C, E1, and E2 proteins) of the disclosure can be produced by transformation or transfection of a suitable host cell with a polypeptide-encoding nucleic acid molecule or fragment thereof in a suitable expression vehicle. In certain aspects, the polypeptide-encoding nucleic acid molecule can comprise a sequence having at least 85%, at least 90%, at least 95%, at least 96%, at least 97%, at least 98%, at least 99%, or 100% identity to SEQ ID NO: 1, SEQ ID NO: 2, SEQ ID NO: 5, SEQ ID NO: 7, SEQ ID NO: 9, SEQ ID NO: 11, SEQ ID NO:13, or any combination thereof.

In certain aspects, the VLPs of the disclosure are produced by transfecting HEK293 cells or cells derived therefrom (e.g., VRC293 cells) with an expression plasmid that encodes for one or more CHIKV structural protein genes (e.g., C, E3, E2, 6K, and/or E1 genes). The expressed proteins can self-assemble into VLPs that are then harvested and purified from the cell culture medium. In certain aspects, the VLPs can be purified by ultrafiltration, diafiltration, and anion exchange chromatography then sterile filtered. In some aspects, the VLPs can be mixed with the appropriate amount of formulation buffer and then adsorbed on sterile aluminum hydroxide adjuvant (e.g., aluminum hydroxide gel (e.g., Alhydrogel®) 2%). In certain aspects, the formulated VLPs do not contain a preservative or antibiotics.

VRC293 cells are derived from human embryonic kidney (HEK) 293 cells (e.g., from ATCC). The HEK293 cells were adapted to grow in suspension culture in animal-free CD293 medium.

In certain aspects, the VLPs disclosed herein are produced by transient transfection of VRC293 cells with a DNA plasmid encoding the structural genes of the CHIKV. In some aspects, after purification, the VLPs can be diluted into a sucrose-containing citrate buffer and filled into single-dose glass vials or pre-filled syringes to form the final Drug Product.

More specifically, the manufacturing process can be divided into the two main stages: upstream production (cell growth and antigen expression), and downstream purification. In the first step, the VRC293 cells can be expanded from the working cell bank (WCB). The cells are then transfected with an expression plasmid that encodes for the CHIKV VLP structural proteins (capsid, E1, E2, E3, and 6K). In some aspects, the expression plasmid comprises the sequence of SEQ ID NO: 1. Certain aspects of the disclosure are directed to a WCB or stable cell line (e.g., HEK293 or HEK293 derived cells) expressing a CHIKV VLP of the disclosure.

In certain aspects, the CHIKV VLP drug substance comprises three recombinant CHIKV structural proteins: (Capsid/core) (about 35 kDa), Envelope 1 (E1) (about 55 kDa) and Envelope 2 (E2) (about 50 kDa). In certain aspects, the three proteins are co-expressed in VRC293 cells and self-assemble to form the enveloped VLPs that are harvested from the culture medium. The CHIKV VLPs are non-infectious and non-replicating but structurally resemble the parent virus with respect to antigen presentation to the immune system. After production in VRC293 cells, the CHIKV VLPs can be purified from the culture medium, e.g., by a combination of column chromatography, ultrafiltration/diafiltration (UF/DF) and direct-flow filtration steps. In certain aspects, the CHIKV VLPs comprise Capsid, E1, and E2 proteins in an amount of at least 95%, at least 96%, or at least 97% of the total CHIKV proteins present in the CHIKV VLPs.

In some aspects, the final CHIKV VLP drug substance comprises a sterile aqueous solution that can be stored at ≤−60° C. In certain aspects, the product is stored at −45° C. to −10° C. In some aspects, the composition or vaccine is formulated as a suspension and stored in a container (e.g., a vial or pre-filled syringe) at 2 to 8° C. (36 to 46° F.) prior to use. In certain aspects, the CHIKV VLP can be stored for at least or up to about 6 hours, at least or up to about 12 hours, at least or up to about 18 hours, or at least or up to about 24 hours at room temperature. In certain aspects, the CHIKV VLP can be stored for at least or up to about 12 hours, at least or up to about 24 hours, at least or up to about 7 days, or at least or up to about two weeks at 2 to 8° C. In certain aspects, the CHIKV VLP can be stored for at least or up to about 3 months, at least or up to about 6 months, at least or up to about 9 months, at least or up to about 12 months, at least or up to about 2 years stored at −45° C. to −10° C.

In certain aspects, a sterile aqueous buffered solution of the CHIKV VLP drug substance can be filled into single dose vials at 40±10 μg/mL. In some aspects, the vials have a nominal fill volume of about 0.8 mL to allow withdrawal of about 0.5 mL.

In some aspects, the drug substance is formulated with an adjuvant and filled into a pre-filled syringe (e.g., a Type I glass luer lock pre-filled syringe). The preparation of the drug product can be performed as an aseptic fill-finish process.

In certain aspects, the pre-filled syringe comprises a composition or vaccine disclosed herein, e.g., a composition or vaccine comprising about 6 μg to about 60 μg (e.g., about 40 μg) of CHIKV VLP, about 100 μg to 500 μg (e.g., about 300 μg) of aluminum (e.g., as aluminum hydroxide adjuvant), about 20 mg to 80 mg (e.g., about 59.7 mg) of sucrose, about 0.2 mg to 0.8 mg (e.g., about 0.4 mg) of potassium phosphate monobasic, about 0.5 mg to 1.5 mg (e.g., about 0.9 mg) of potassium phosphate dibasic, about 1 mg to 10 mg (e.g., about 5.9 mg) of sodium citrate dihydrate, and water for injection.

### Kits

Certain aspects of the disclosure are directed to a pharmaceutical kit comprising one or more containers filled with one or more of the ingredients of the composition, vaccine, or dosage formulations disclosed herein. In some aspects, the kit comprises two containers, one comprising VLPs and the other comprising an adjuvant. In some aspects, the kit comprises a single container (e.g., a pre-filled syringe or vial) comprising VLPs and, optionally, an adjuvant. Associated with such container(s) can be information in the form prescribed by a governmental agency regulating the manufacture, use or sale of pharmaceuticals or biological products, which notice reflects approval by the agency of manufacture, use or sale for human administration.

In certain aspects, a composition, vaccine and/or dosage form (e.g., a one-time single dose, a prime dose or a boosting dose) is supplied as a liquid, e.g., a suspension in an appropriate concentration for administration to a subject (e.g., intramuscular administration).

Certain aspects of the disclosure are directed to a kit comprising a composition, a vaccine, and/or a dosage form of the disclosure (e.g., in a pre-filled syringe), and instructions for use.

In certain aspects, the kit comprises a first container comprising a VLP-containing composition, vaccine and/or dosage form disclosed herein and a second container comprising an adjuvant (e.g., an aluminum adjuvant), and instructions for combining the contents of the containers and administering the mixture to a subject.

In certain aspects, the kit comprises a container (e.g., a vial or a pre-filled syringe) comprising a composition, vaccine and/or dosage form disclosed herein which comprises a VLP and an adjuvant (e.g., an aluminum adjuvant), and instructions for administering the composition, vaccine and/or dosage form to a subject.

In certain aspects, the kit comprises a sterile suspension for intramuscular administration available in a vial or a single dose pre-filled syringe (comprising a volume of about 0.5 to 1.5 mL (e.g., about 0.8 mL). In certain aspects, each dose comprises about 6 μg to about 60 μg (e.g., about 40 μg) of CHIKV VLP, about 100 μg to 500 μg (e.g., about 300 μg) of aluminum (e.g., as aluminum hydroxide adjuvant), about 20 mg to 80 mg (e.g., about 59.7 mg) of sucrose, about 0.2 mg to 0.8 mg (e.g., about 0.4 mg) of potassium phosphate monobasic, about 0.5 mg to 1.5 mg (e.g., about 0.9 mg) of potassium phosphate dibasic, about 1 mg to 10 mg (e.g., about 5.9 mg) of sodium citrate dihydrate, and water for injection.

### EXAMPLES

### Example 1: Preparation of a Chikungunya Virus-Like Particles (VLPs)

A plasmid containing the coding sequence for CHIKV structural proteins (capsid (C), E3, E2, 6K, and E1) was prepared. The plasmid was prepared from a 3744 base pair synthetic gene (SEQ ID NO: 2) coding for the five CHIKV structural genes: Capsid (core), E3, E2, 6K, and E1 of strain 37997. The CHIKV genes were inserted into the plasmid in the same orientation as in the CHIKV genome. The synthetic gene was constructed with XbaI and BamHI restriction endonuclease site. This gene was inserted into the multi cloning site of the expression vector by digesting the vector with the XbaI and BamHI restriction endonucleases. This vector utilizes the human cytomegalovirus (CMV) early immediate promoter/enhancer and the regulatory R region from the 5′ long terminal repeat of human T cell leukemia virus type 1 for efficient and high level expression of the gene of interest in human cells.

The VLPs in this Example were produced by transfecting VRC293 cells (derived from human embryonic kidney (HEK) 293 cells) with the expression plasmid that encodes the CHIKV structural protein genes (C, E3, E2, 6K, and E1 genes). The expressed proteins self-assemble into VLPs that were then harvested and purified from the cell culture medium. The proteins were expressed under the control of a CMV IE promoter. The 6K peptide was incorporated into alphavirus virions in small amounts, but was not identified in the virion structure by cryoelectron microscopy. The 6K and E3 proteins were not detected in the purified CHIKV VLPs by western blot or other techniques.

The VLPs were purified by ultrafiltration, diafiltration, and anion exchange chromatography then sterile filtered. In short, the initial purification step was an Ultrafiltration/Diafiltration (UF/DF) process to concentrate the CHIKV VLPs and then diafilter the concentrated material into 1XSP buffer (10 mM potassium phosphate, 218 mM sucrose, pH 7.0) for further purification, while removing impurities from the clarified harvest. This process was performed by tangential flow filtration (TFF).

Next, the concentrated and diafiltered CHIKV VLPs were applied to an anion-exchange chromatography column. This step removed host cell protein and host cell deoxyribonucleic (DNA). During the chromatography process, there was a wash step to reduce the size and concentration of residual host cell DNA in the product. The CHIKV VLPs were eluted from the column by increasing the ionic strength of the buffer with 25 mM sodium citrate. The process was performed at room temperature.

Next, the CHIKV VLPs eluted from the anion-exchange column were diafiltered against 5-7 volumes of 1XSP buffer. The diafiltered CHIKV VLP material was recirculated for 3-15 minutes with no transmembrane pressure (TMP) and then collected. This step served to remove any remaining residual wash solution and to lower the level of other host cell impurities. Finally, following the diafiltration, the CHIKV VLP product was membrane filtered into a sterile, single-use container to form the drug substance. Samples were collected for release testing and the CHIKV VLP drug substance was stored at ≤−60° C.

### Example 2: Characterization of a Chikungunya Virus-Like Particles (VLPs)

The size of the CHIKV VLPs prepared according to the methods in Example 1 was determined using DLS, also referred to as Photon Correlation Spectroscopy (PCS) or Quasi-Elastic Light Scattering, which is a well-established technique for measuring the size of molecules and particles in the submicron size range. The parameter measured by DLS is the hydrodynamic radius and can be defined as the radius of a spherical particle that has the same diffusion coefficient as the particle being measured. These studies were performed with a Zetasizer Nano (Malvern). The diameter range of particles measured by this instrument is 0.6 nm to 6 μm. DLS measurements were performed using disposable low volume cuvettes. For these studies, samples from six lots of drug substance were directly added to the cuvettes without dilution. The mean diameter results for the 6 lots are shown in Table 2. The z-average size, corresponding to the mean diameter of the main peak was 63-67 nm. The low PDI indicates that purified VLPs were relatively homogenous in size. The CHIKV VLP size obtained by DLS was consistent with the value obtained by cryoelectron microscopy (40-60 nm) and TEM (70 nm) that was performed on development lots. These results also agree with the known size of alphavirus virions which are in the range of 60-70 nm.

TABLE 2
Assessment of CHIKV VLP Particle Size by DLS

| Diameter (nm) | Polydispersity Index (PDI) |
|---|---|
| 66.6 | 0.03 |
| 67.7 | 0.05 |
| 67.1 | 0.03 |
| 67.6 | 0.04 |
| 63.9 | 0.03 |
| 63.7 | 0.04 |

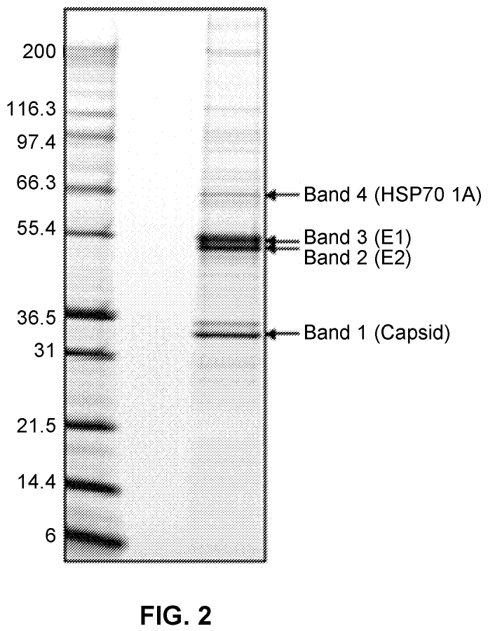

Next, SDS-PAGE analysis of CHIK VLPs under reducing and denaturing conditions was conducted and showed 3 major bands. A MALDI-TOF mass spectroscopy method was used to confirm that the 3 major bands shown as 1, 2, and 3 in FIG. 2 corresponded to the 3 structural proteins: Capsid, E2, and E1, respectively, from the chikungunya virus. The minor contaminant slightly larger than E1 (running above the E1) in SDS-PAGE shown as band 4 in FIG. 2 was determined to be HSP70 1A.

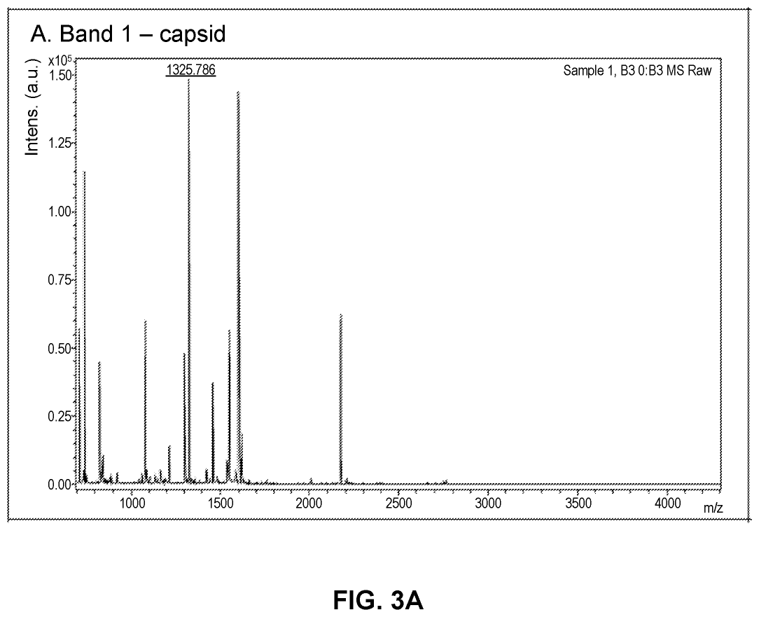

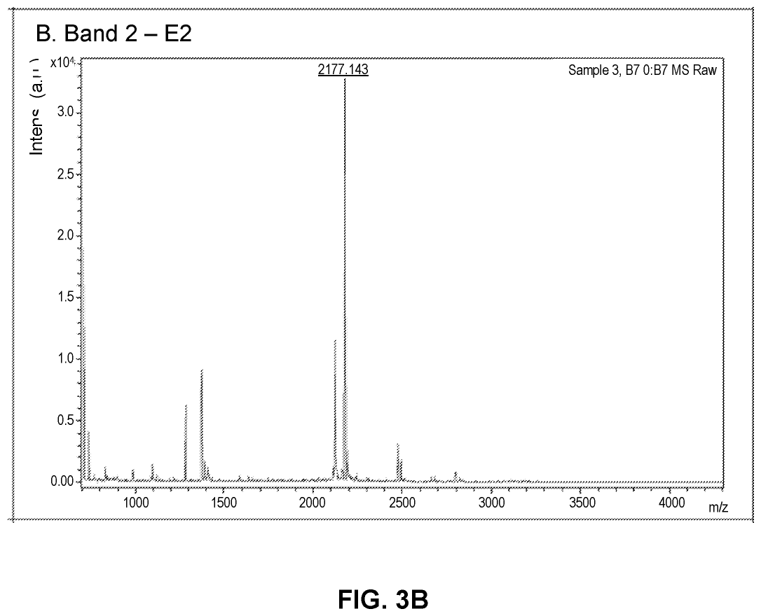

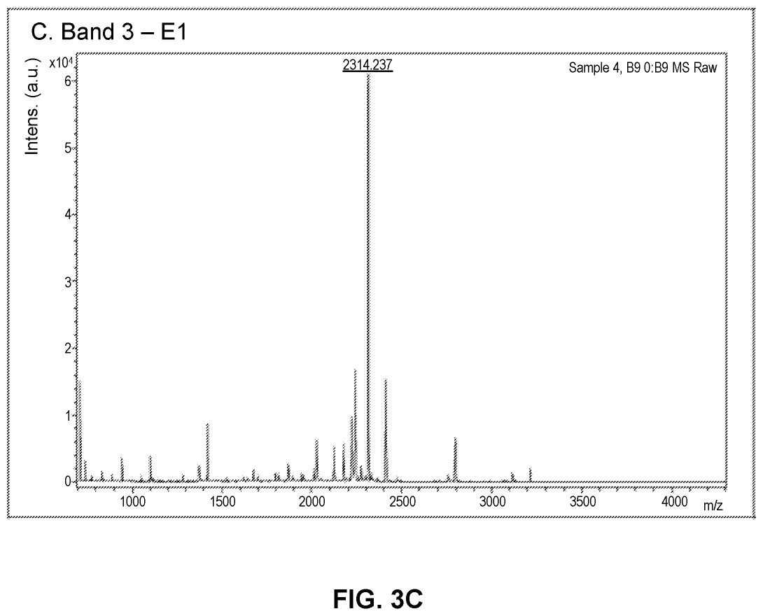

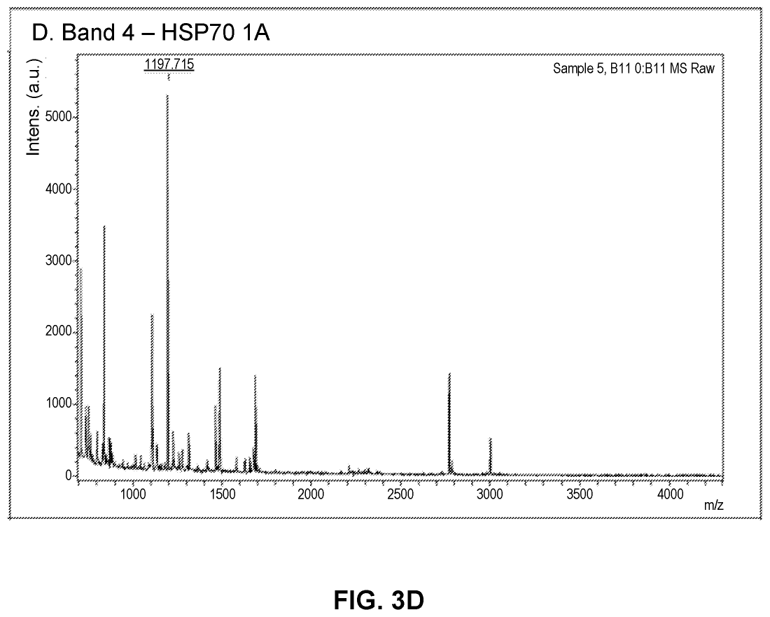

The MS spectroscopy data obtained from trypsinized Capsid, E1, and E2 bands are shown in FIGS. 3A-3C. In addition, the MS spectroscopy data obtained for host cell protein (HSP70 1A) is shown in FIG. 3D. Sequence assignments of the fragments obtained for Capsid, E1, and E2 structural proteins were achieved by matching the measured masses with the expected masses.

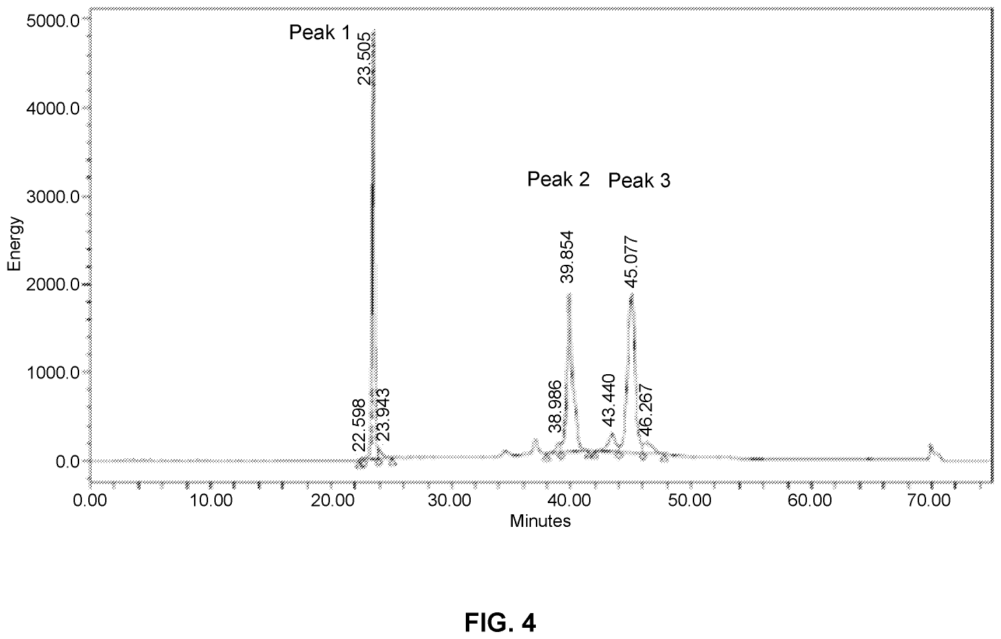

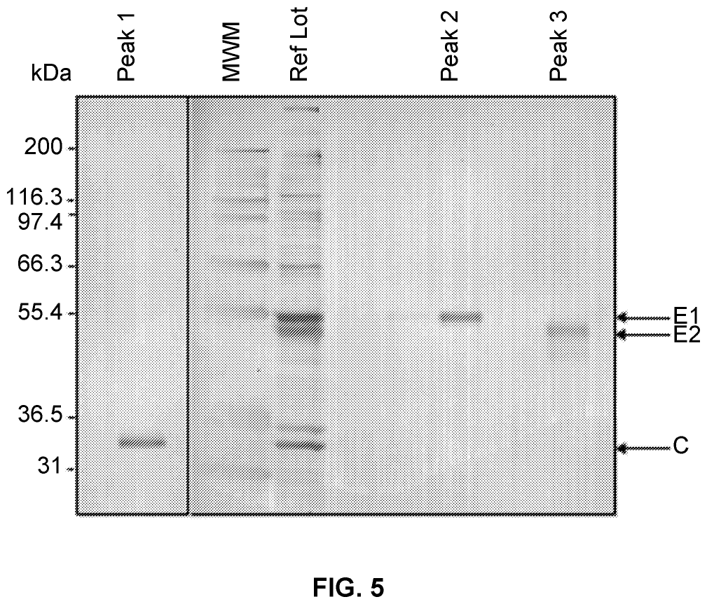

An RP-HPLC assay was developed to determine the percentage of structural proteins E1, E2, and Capsid in CHIKV VLP. Aliquots of 100 μL undiluted CHIKV VLP were loaded onto an Xbridge BEH300 C4 HPLC column (3.5 μm, 4.6×150 mm). The column was maintained at 60° C. The HPLC Mobile Phase A contained water with 0.1% TFA, and Mobile Phase B contained 30% acetonitrile, 70% isopropanol and 0.03% TFA. Proteins were eluted with a gradient of Mobile Phase B from 0% to 100% over 60 minutes with a flow rate of 1 mL/min. The eluted proteins were detected by using a fluorescence detector with excitation wavelength of 280 nm and emission wavelength of 350 nm. FIG. 4 shows the RP-HPLC profile of 100 μL injection of undiluted CHIKV VLP. The 3 major peaks obtained by RP-HPLC analysis were identified to be Capsid, E1, and E2 by SDSPAGE (FIG. 5). The RP-HPLC peaks with retention time of 23.6 (Peak 1), 40.0 (Peak 2), and 45.2 min (Peak 3) were collected.

It was confirmed that the three peaks were Capsid, E1, and E2, and they constituted >97% of the CHIKV proteins. The percentage of each of the Capsid, E1, and E2 proteins was calculated by dividing the corresponding protein peak area with the total peak area of C+E1+E2. The observed percentage of C, E1, and E2 for 100 μL of undiluted CHIK VLP was 35%, 29%, and 36%, respectively. The calculated C, E1, and E2 percentages were derived from the fluorescence signal of each protein component under denaturing condition. These values are close to a 1:1:1 ratio of Capsid, E1 and E2.

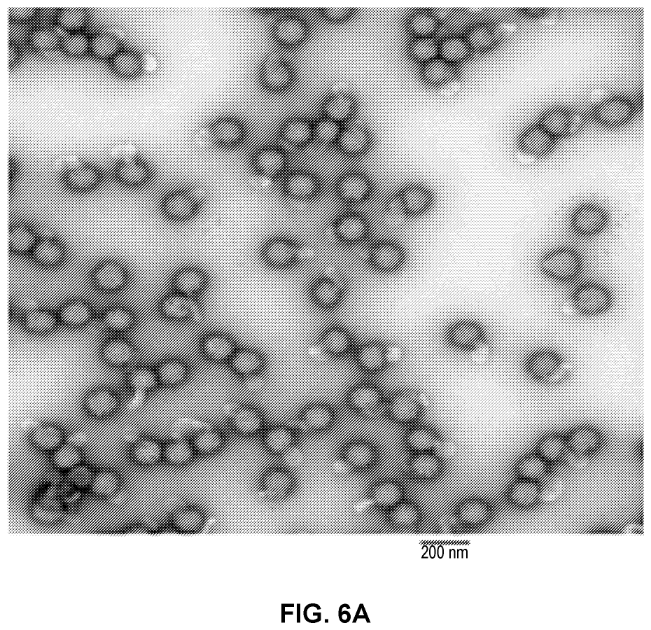

Negative-stain electron microscopy was used to analyze the morphology of the purified CHIKV VLPs. For these studies, samples were prepared by mixing undiluted CHIKV VLPs with a known concentration of 100 nm latex beads, and applying the mixture to nitrocellulose supported 400 mesh copper grids. The sample was allowed to attach to the grid, which was then stained with phosphotungstic acid solution (sodium salt). The grids were analyzed by electron microscopy using an FEI Tecnai T12 electron microscope operating at 120 KeV equipped with an FEI Eagle 4K×4K CCD camera. Images were taken at a nominal magnification of 15,000× (0.71 nm/pixel). The most prominent structure, representing >95% of the total particles, corresponds to an intact CHIKV VLP that has a morphology similar to chikungunya virus (FIG. 6A). The intact CHIKV VLPs were approximately spherical in shape with a brushy border and a solid center. The size of the CHIKV VLPs correlated well with the known size of alphavirus, which has virions in the range of 60-70 nm.

With cryoelectron microscopy, samples were viewed in a hydrated state in the absence of stains or fixatives that are required for traditional electron microscopy. Samples of CHIKV VLPs were prepared for cryoelectron microscopy by flash-freezing them in vitreous ice supported by holey carbon films on 400 mesh carbon and stored under liquid nitrogen until imaging. The vitreous ice grids were transferred to an electron microscope (FEI Tecnai T12, 120 KeV, FEI Eagle 4K×4K CCD camera) using a stage that maintains the grids at ≤−170° C. Images of each grid were acquired at nominal magnifications of 52,000× (0.21 nm/pixel) and 21,000× (0.50 nm/pixel). Examination of the CHIKV VLPs by cryoelectron microscopy analysis (FIG. 6B) demonstrated that the preparation included virus-like particles that were uniform in size and shape with a brushy border, and appear to possess a high level of symmetry. The diameter of the particles was estimated to be 40-60 nm depending upon the structure of the VLPs. Naked VLPs (VLPs without the envelope spikes) were smaller, i.e., ≈40 nm, and fully formed mature VLPs were ≈60 nm. The morphology of these particles was suitable for 2D averaging and potentially for 3D reconstruction. The E1 and E2 glycoproteins were organized into 240 heterodimers that in turn are assembled into 80 glycoprotein spikes.

The N-glycan profile of the CHIK VLP was determined by derivatizing the PNGase cleaved glycans with 2-aminobenzamide (2AB) and separating the derivatized glycans on a normal-phase (NP) HPLC with fluorescence detection. LC-MS analysis has identified oligomannose, hybrid, and complex N-linked glycan structures in CHIK VLP.

A sandwich-type ELISA that measures the concentration and/or confirmation of a neutralizing epitope on the CHIKV VLP structure was developed for use as a potency assay. This assay used two different monoclonal antibodies for capture and detection. These antibodies, capture and detection, were produced by immunization of mice with purified VLPs and screening of hybridomas against purified VLPs in an ELISA. Antibodies that recognized VLPs in the primary screening by ELISA were subsequently analyzed for their ability to neutralize CHIKV VLPs using pseudotyped lentiviral vectors in a reporter gene-based neutralization assay. Both capture and detection antibodies are neutralizing antibodies. The detecting antibody was conjugated to HRP. The antigenicity of a reference standard lot, and six CHIKV VLP samples was determined by ELISA. Absorbance was determined at 450 nm using a Spectramax M5 plate reader. The resulting optical densities were plotted as a function of the protein concentration and a standard curve was generated.

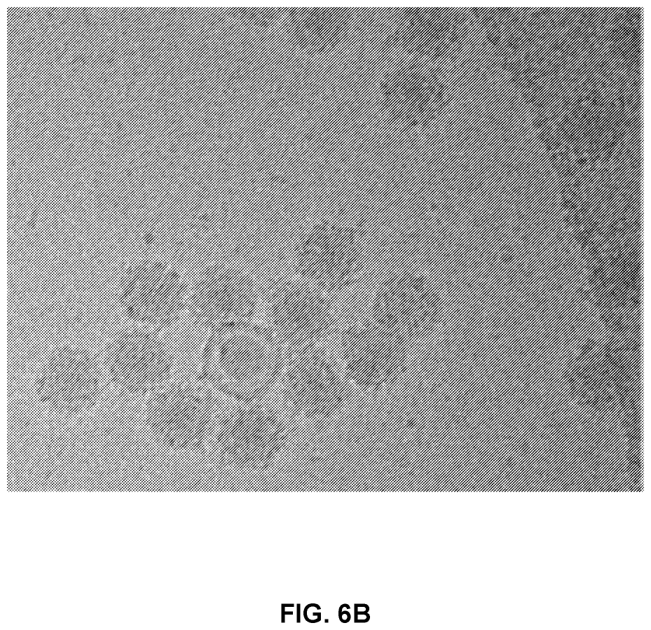

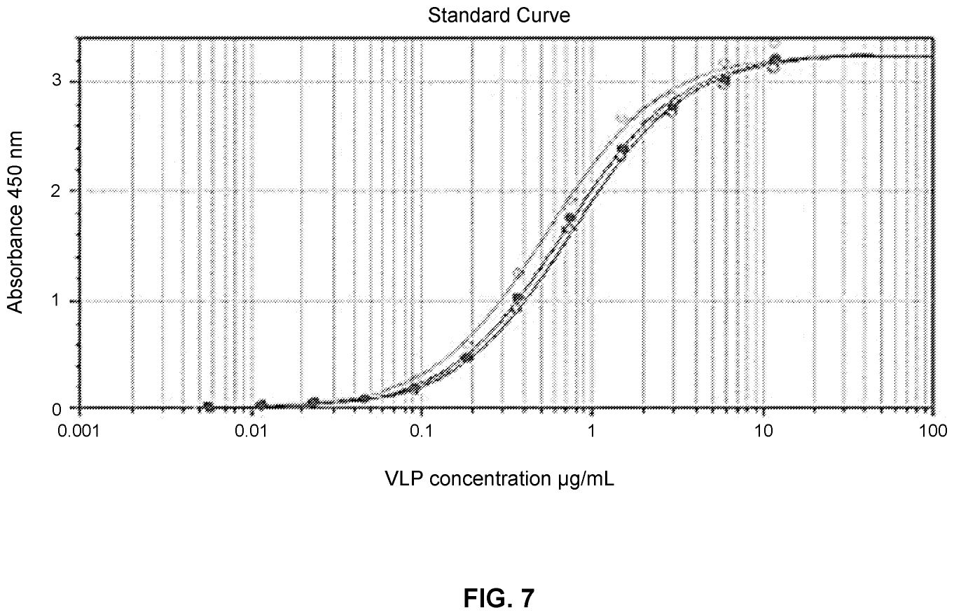

The results for the reference standard lot and two GMP lots are presented in FIG. 7. When the optical density was plotted as a function of the CHIKV VLP concentration, the dilution curves for all lots had similar slopes and the dilution at which a 50% response was observed. These data suggest the CHIKV VLPs produced by the current manufacturing process demonstrated good lot-to-lot consistency with respect to antigenicity.

Similarly, the dose-response curve generated for the reference standard lot was used to calculate a potency (CHIKV VLP immunoreactivity) value for each of four GMP lots of CHIKV VLP. For each lot, the potency (immunoreactivity) value was determined from the standard curve using single dilution that fell within the linear range of the standard curve. These results are presented in Table 3 along with the protein concentration of each lot and the ratio of the potency (CHIKV VLP immunoreactivity) by ELISA: Protein concentration by BCA™. The potency (immunoreactivity) of the reference standard lot was assigned a value equivalent to the protein concentration, measured by BCA™. The ratio of the ELISA value to protein concentration provides a measure of potency (immunoreactivity) per unit of mass.

TABLE 3
Potency (Immunoreactivity) of CHIKV VLP Drug Substance Lots

| | Potency | Protein | Ratio of Potency |
|---|---|---|---|
| | (Immunoreactivity) | concentration | (Immunoreactivity) to |
| Lot | by ELISA | by BCA ™ | Protein Concentration |
| No. | (μg/mL) | (μg/mL) | (ELISA:BCA ™) |
| 1 | 42.7 | 55 | 0.8 |
| 2 | 58.9 | 51 | 1.2 |
| 3 | 33.8 | 41 | 0.8 |
| 4 | 44.0 | 51 | 0.9 |
| 5 | 85.4 | 74 | 1.2 |
| 6 | 85.0 | 76 | 1.1 |

### Example 3: Efficacy of the CHIKV VLP in Cynomolgus Macaques

A non-human primate (NHP) animal model of CHIKV disease (cynomolgus macaques challenged with CHIKV strain LR2006-OPY1) was used to evaluate CHIKV VLP vaccine, e.g., immunogenicity and efficacy. In short, NHP were challenged with three doses of CHIKV LR2006-OPY1 (10⁶, 10⁷, 10⁸ plaque forming units (PFU)). All three doses induced clinical symptoms including viremia, arthralgia, fever, and anorexia within 10 days of challenge. Viremia was measured by both plaque assay (for the detection of infectious virus) and qRT-PCR (for the detection of viral RNA) in the plasma and tissue of infected animals daily for 7 days following challenge and on the day of euthanasia, 10 days post-challenge. While there were no outward signs of arthralgia in the challenged animals during the 10-day monitoring period, inflammation of the joints and surrounding tissues were apparent upon histological examination. A scoring system was developed to measure the degree of inflammation and degeneration observed (Table 4). There was no significant difference in the disease parameters measured between the three doses. Based on the similarities in viremia and joint effusion between the three doses, and the increased need for supportive care at the highest dose, the dose of 10⁷ PFU was selected for future studies.

TABLE 4
Scoring System for Joint and Synovium Pathology in CHIKV-Infected
Cynomolgus Macaques

| Observation | Grade | Description |
|---|---|---|
| Inflammation | 1 | Minimal inflammation = Few scattered individual inflammatory cells |
| | | or aggregates of less than 10 cells per aggregate |
| | 2 | Mild inflammation = Inflammation forms small aggregates of 10-50 |
| | | cells per aggregate |
| | 3 | Moderate inflammation = Inflammatory cells form aggregates of |
| | | more than 50 cells per aggregate |
| Fibrin | 1 | Add additional point for presence of fibrin/necrosis |
| Neutrophils | 1 | Add additional point for presence of neutrophils |

Cynomolgus macaques were immunized twice with CHIKV VLP (1.25, 6, or 20 μg) adjuvanted with alum (300 μg), VLP alone (20 μg), or alum alone, followed by challenge with CHIKV LR2006-OPY1 (10⁷ PFU).

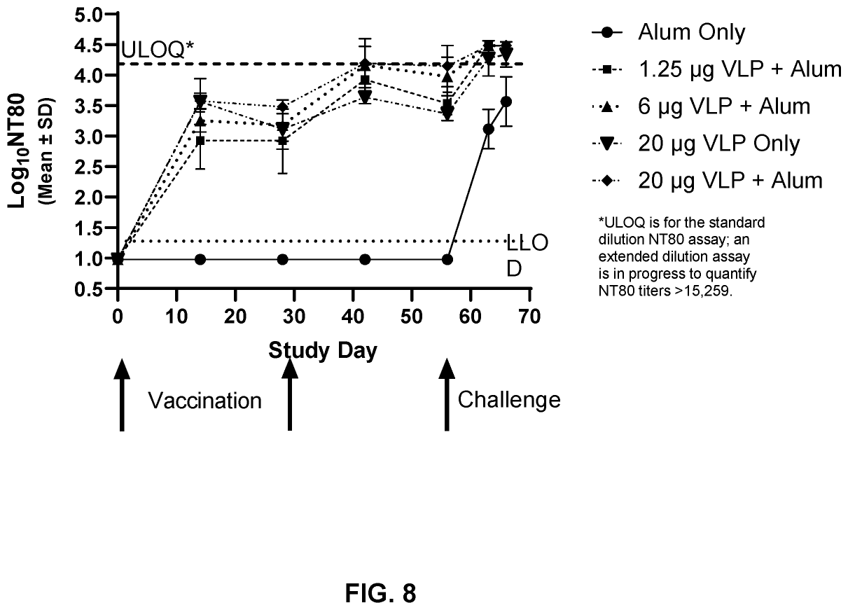

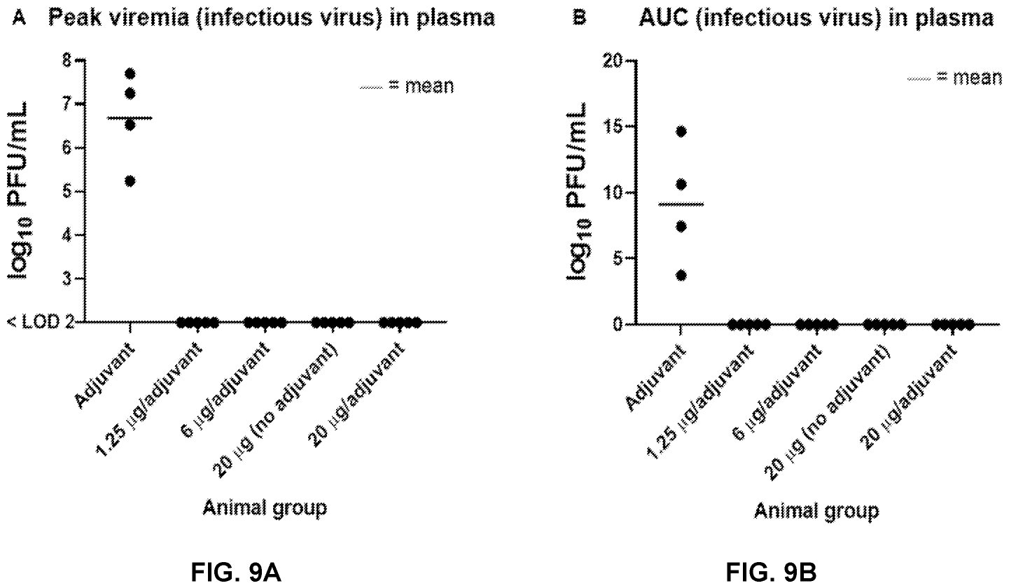

The results showed that all three doses of the VLP, including 20 μg without alum, induced an immune response, as measured by the infectivity inhibition assay (CHIKV-Luc) quantitated by the decrease in luciferase activity. Strong serum neutralizing antibody (SNA) response was observed after a single immunization (FIG. 8). The addition of alum increased the pre-challenge SNA titers compared to the VLP vaccine without alum (20 dose; see FIG. 8). Furthermore, vaccination protected non-human primates (NHPs) from viremia as measured by plaque assay on serum from challenged animals (FIGS. 9A-9B). No infectious virus was observed at any time point in vaccinated animals.

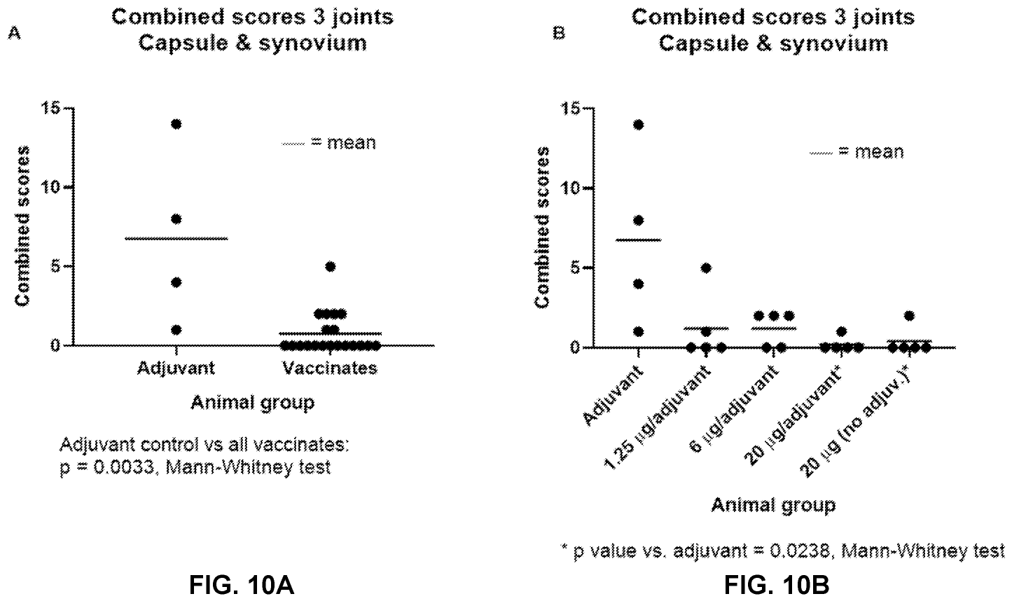

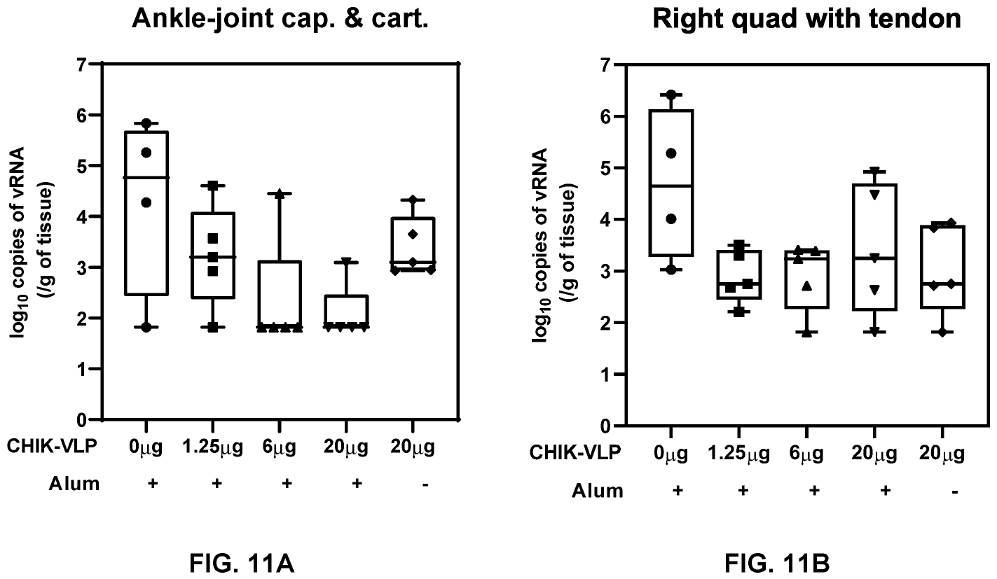

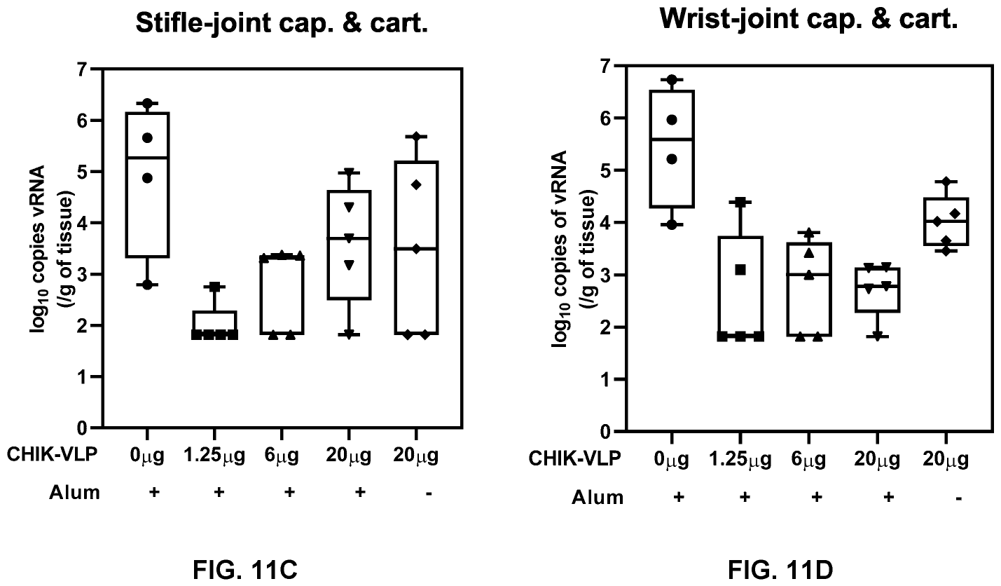

Joint pathology scores demonstrate that vaccination with the VLPs protects NHPs from joint infiltration in a dose-dependent manner (FIGS. 10A-10B). By RT-PCR analysis, 3 of the 4 control NHPs were shown to have measurable RNA in all of the tissues analyzed whereas the NHPs vaccinated with the VLPs had significantly less viral RNA (FIGS. 11A-11D and Table 5). The vRNA/g of tissue was significantly higher in animals that received alum only (0 μg CHIKV-VLP+alum) compared with vaccine recipients (1.25, 6, 20 CHIKV-VLP+alum, 20 μg CHIKV-VLP). In this NHP study, there was no difference observed between the different active CHIKV VLP vaccine groups. Taken together, these data suggest that vaccination-induced SNA protects NHPs from developing chikungunya disease following viral challenge.

TABLE 5
Individual SNA Titres Joint Pathology Scores and Tissue Log Copies
vRNA/g

| | | Joint Pathology Score | | | | | |
|---|---|---|---|---|---|---|---|
| | | Capsule/ | | | Joint Tissue Log Copies | | |
| Animal | | synovium | | Day 56 | VRNA/g | | |
| ID | Test Group | only | Combined¹ | Titre | Stifle | Ankle | Wrist |
| 47567 | Adjuvant | 1 | 1 | <19 | 2.792 | <LOQ | 3.958 |
| 47571 | only | 14 | 23 | <19 | 5.660 | 5.830 | 6.730 |
| 47580 | | 8 | 8 | <19 | 6.329 | 5.255 | 5.215 |
| 47590 | | 4 | 4 | <19 | 4.873 | 4.273 | 5.966 |
| 47562 | 1.25 μg | 0 | 0 | 2979.1 | <LOQ | 4.605 | 4.387 |
| 47563 | VLP with | 5 | 6 | 3356.1 | 2.752 | 3.197 | 3.097 |
| 47574 | adjuvant | 0 | 0 | 2163.4 | <LOQ | 3.567 | <LOQ |
| 47588 | | 0 | 0 | 3138.2 | <LOQ | 2.923 | <LOQ |
| 47589 | | 1 | 1 | 6891.4 | <LOQ | <LOQ | <LOQ |
| 47560 | 6 μg VLP | 0 | 0 | 5218.5 | <LOQ | <LOQ | <LOQ |
| 47577 | with | 2 | 2 | 12147.2 | 3.363 | <LOQ | <LOQ |
| 47587 | adjuvant | 2 | 2 | >15259 | 3.317 | 4.450 | 3.423 |
| 47596 | | 0 | 0 | 6283.4 | <LOQ | <LOQ | 3.004 |
| 47597 | | 2 | 2 | 6366 | 3.374 | <LOQ | 3.812 |
| 47570 | 20 μg (no | 0 | 0 | 1726.4 | <LOQ | 2.946 | 4.021 |
| 47573 | adjuvant) | 0 | 0 | 2157.2 | 5.681 | 3.095 | 4.170 |
| 47575 | | 0 | 0 | 2027.3 | 4.744 | 4.326 | 4.783 |
| 47591 | | 0 | 0 | 2730.7 | 3.494 | 2.926 | 3.653 |
| 47598 | | 2 | 2 | 3353.6 | <LOQ | 3.652 | 3.456 |
| 47561 | 20 μg VLP | 0 | 0 | >15259 | 4.299 | <LOQ | 3.127 |
| 47564 | with | 0 | 0 | 5442.8 | <LOQ | 3.092 | <LOQ |
| 47565 | adjuvant | 1 | 1 | 13454.3 | 3.692 | <LOQ | 3.145 |
| 47579 | | 0 | 0 | 7838.9 | 3.171 | <LOQ | 2.728 |
| 47586 | | 0 | 0 | >15259 | 4.971 | <LOQ | 2.781 |

¹Includes tendon for the ankle and wrist; includes tendon and meniscus for the stifle.

### Example 4: Safety and Immunogenicity of CHIKV VLP in Humans (Phase 2)

A phase 2 clinical trial was developed to examine the safety and immunogenicity of the CHIKV VLP vaccine in humans. 400 patients were selected that were between 18 and 60 years of age. The trial was randomized, double-blind, placebo-controlled, and the groups were studied in parallel. The patients were either CHIKV-naïve or CHIKV-exposed and from CHIK-endemic areas in the Caribbean (Puerto Rico, Dominican Republic, Haiti, Martinique, and Gaudeloupe). CHIKV-naïve patients were categorized as patients whose titer was <15 at week 0. Patients received 20 μg of CHIKV-VLP intramuscularly at weeks 0 and 4. The primary endpoint was seroconversion at week 8 with an 18-month follow-up (through two CHIKV seasons).

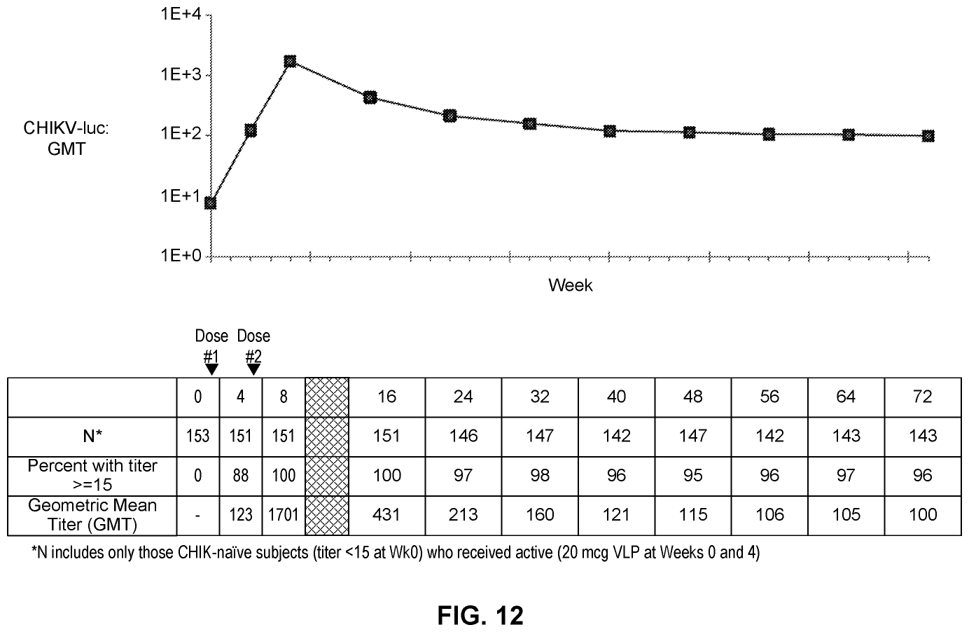

For this study, a baseline serum neutralizing antibody (SNA) was detected in about 30% of subjects in the treatment and the placebo groups. High response rates were observed in subjects who had no SNA at baseline. 20 μg CHIKV VLP induced an antibody response comparable to a response to natural antigen. 95% CI of titers in CHIK-VLP group at week 8 overlapped with range of baseline titers in CHIKV-exposed. At 8 weeks, the CHIKV-VLP treated patients had a 99.5% response rate compared to the 20.8% response rate for placebo, p-value<0.001. Positive immune response was defined as SFV-OPY1-GFP EC50 value greater than or equal to 30. At 8 weeks, the CHIKV-VLP treated patients had a 2004.5 (1680.1-2391.5) GMT compared to 42.8 (31.7-57.9) GMT, p-value<0.001. Patients maintained an immune response through 72 weeks after administration of the vaccine (FIG. 12 and Table 6). No apparent safety differences between CHIKV-naïve and CHIKV-exposed were observed.

TABLE 6
Titer of CHIKV-naïve patients that were administered the CHIKV VLP.

| | Weeks After Administration | | | | | | | | | | |
|---|---|---|---|---|---|---|---|---|---|---|---|
| | 0 | 4 | | | | | | | | | |
| | (first | (second | | | | | | | | | |
| | dose) | dose) | 8 | 16 | 24 | 32 | 40 | 48 | 56 | 64 | 72 |
| N | 153 | 151 | 151 | 151 | 146 | 147 | 142 | 147 | 142 | 143 | 143 |
| Percent | 0 | 88 | 100 | 100 | 97 | 98 | 96 | 95 | 96 | 97 | 96 |
| with titer | | | | | | | | | | | |
| >=15 | | | | | | | | | | | |
| Geometric | — | 123 | 1701 | 431 | 213 | 160 | 121 | 115 | 106 | 105 | 100 |
| Mean | | | | | | | | | | | |
| Titer | | | | | | | | | | | |

### Example 5: CHIKV VLP Vaccine Stability

The CHIKV VLP vaccine was prepared as a sterile aqueous buffered solution that was filled in a single dose 3 mL glass vial with 0.8 mL of vaccine at a concentration of 40+/−10 μg/mL. The formulation buffer included 218 mM sucrose, 10 mM potassium phosphate, 25 mM sodium citrate, and water for injection. The vial was stored in a non-frost-free freezer at −45° C. to −10° C. and was intended for intramuscular administration. Vials were intended for single use only and did not contain a preservative.

Results from a stability study conducted show that the vaccine was stable in a syringe for 8 hours at refrigerated and room temperature (data not shown).

Stability results through 6 months from a toxicology lot stored at −45° C. to −10° C. were also obtained. Samples were analyzed by appearance, pH, protein concentration, SDS-PAGE, western blot, ELISA, and dynamic light scattering and are analyzed at 3-month intervals up to 1 year, followed by every 6 months up to 2 years, then annually thereafter. In addition, container closure integrity is analyzed yearly. No evidence of product instability or degradation was observed through the 6-month time point.

### Example 6: CHIKV VLP Vaccine Induces Neutralizing Antibody Response in Humans

An additional Phase 2 clinical study was designed with 1- and 2-dose regimens using 6 to 40 μg doses of CHIKV VLP with and without alum. In this study, 415 patients between the ages of 18-45 were randomized across eight treatment groups. A Group 9 with plasmapheresis and leukapheresis was also included (data not shown here, see Example 8).

Group 1: an unadjuvanted 20 μg dose on a standard 2-dose schedule at Day 1 and Day 29.

Groups 2-7: various adjuvanted doses (6, 10, and 20 μg) of CHIKV VLP on a standard or short (Day 15 and Day 29) 2-dose schedule.

Group 8: a single adjuvanted 40 μg dose on Day 29.

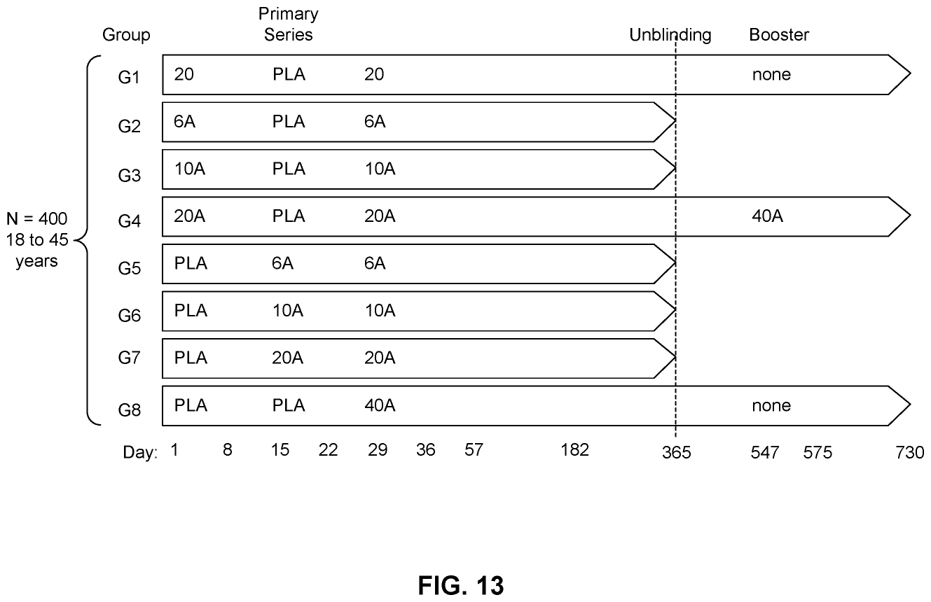

To maintain blinding across treatment groups, placebo was administered at day 1 for subjects on the short and single dose schedules and at day 15 for subjects on the standard and single dose schedules. All subjects received the CHIKV VLP at Day 29. A summary of the study design is shown in FIG. 13 and Table 7.

TABLE 7
CHIK VLP With and Without Alum Dose and Schedule Study Design

| | | Dose | | | | Final Visit | Booster |
|---|---|---|---|---|---|---|---|
| | | Level/μg | Initial Dose Regimen | | | (Discontinued | Dose |
| Group | N | VLP | Day 1 | Day 15 | Day 29 | Groups) | Day 547 |
| 1 | 53 | 20 | 20 μg/unadj. | Placebo | 20 μg/unadj. | — | None |
| 2 | 52 | 6 | 6 μg/adj. | Placebo | 6 μg/adj. | Day 365 | |
| 3 | 52 | 10 | 10 μg/adj. | Placebo | 10 μg/adj. | Day 365 | |
| 4 | 50 | 20 | 20 μg/adj. | Placebo | 20 μg/adj. | — | 40 μg/adj. |
| 5 | 53 | 6 | Placebo | 6 μg/adj. | 6 μg/adj. | Day 365 | |
| 6 | 53 | 10 | Placebo | 10 μg/adj. | 10 μg/adj. | Day 365 | |
| 7 | 51 | 20 | Placebo | 20 μg/adj. | 20 μg/adj. | Day 365 | |
| 8 | 51 | 40 | Placebo | Placebo | 40 μg/adj. | — | None |
| 9 | 20 | 20 | 20 μg/adj. | — | 20 μg/adj. | Day 182 | |
| | | | | | | apheresis arm | |

Unadj. = unadjuvanted;
Adj. = adjuvanted with 300 μg Alhydrogel ®

Immunogenicity analyses were performed on a modified intent-to-treat (mITT) population (n=412), which included all randomized subjects who were treated and had evaluable immunogenicity results for baseline (Day 1) and at least 1 on-study sample.

Serum neutralizing antibodies (SNAs) was assessed in all groups at Days 1, 8, 15, 22, 29, 36, 57, 182, and 365, using the CHIKV-Luc luciferase-based microneutralization assay. Titers were determined using a cut-off of 80% neutralization (NT80). Geometric mean titers (GMTs) were calculated at each time point, along with the percent of subjects exhibiting a NT80 at or above thresholds of 15, 40, 60, 80, 100, 160, and 640. Statistical comparison was performed between Group 1 and all other groups at all post-Day 1 time points by an analysis of variance for the GMTs and a Fisher's exact test for the categorical percentage of subjects reaching the various threshold definitions.

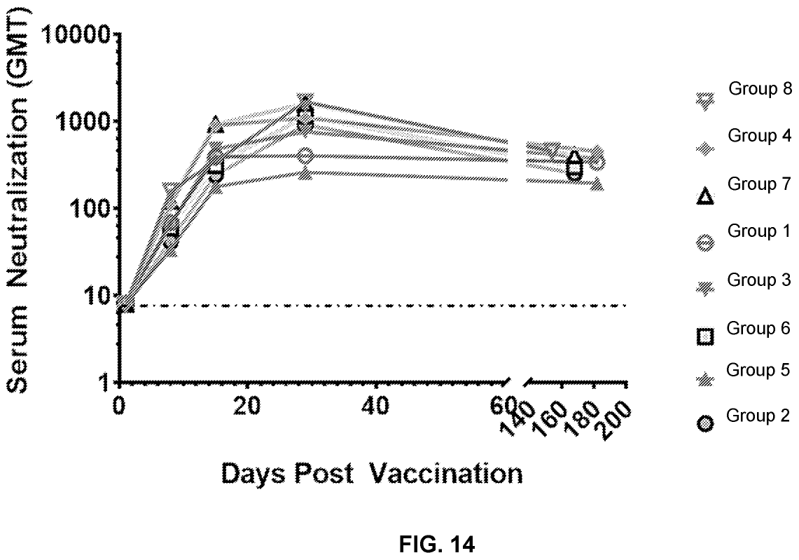

The treatments were well tolerated across the study, and no significant vaccine-related safety concerns were identified. The second interim analysis of patients post-vaccination shows that up to 98% of patients produced a neutralizing antibody response against CHIKV by Day 8 (within 7 days) (FIG. 14). Seroconversion was in 74% to 98% of subjects within 7 days after one dose. Seroconversion was observed in all subjects by 28 days after the last dose. This immune response was shown to be persistent through the six-month visit.

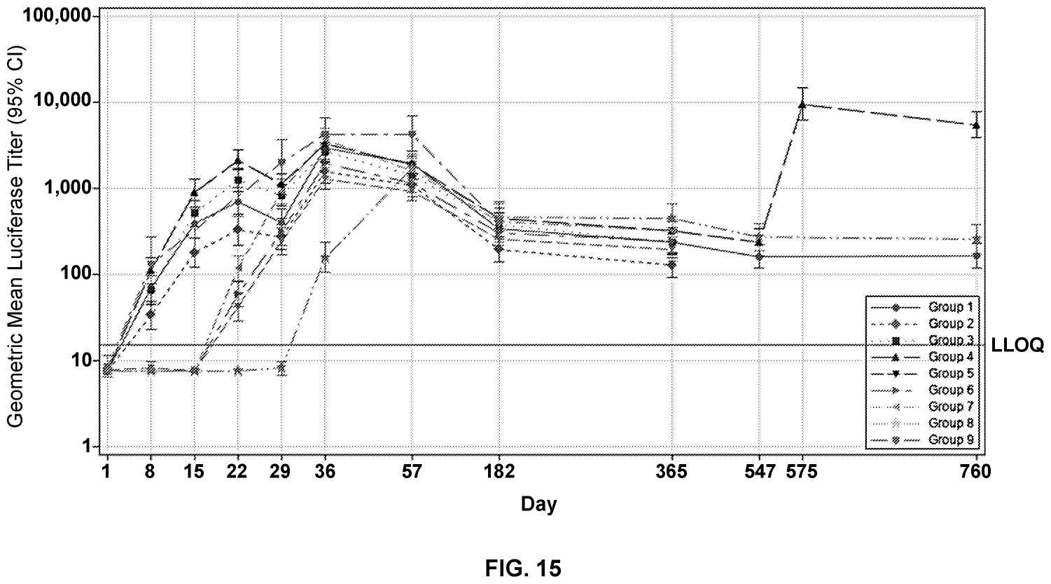

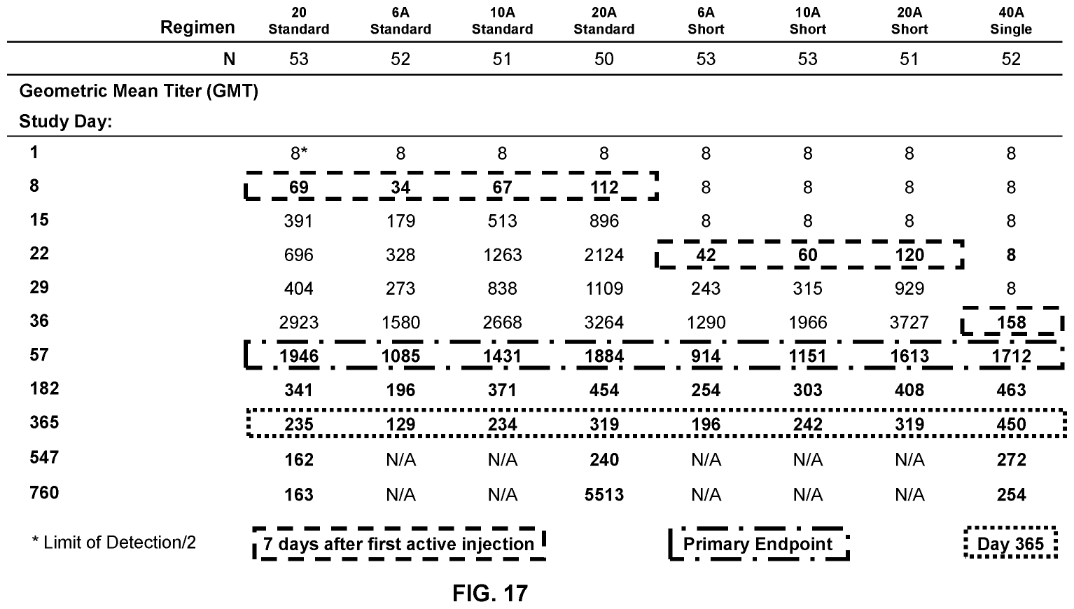

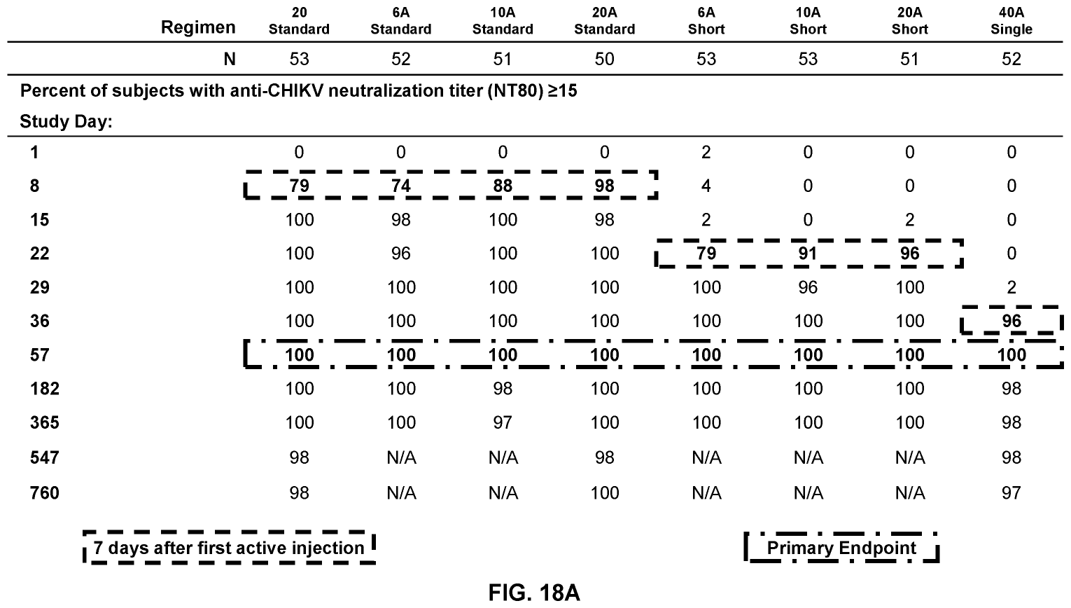

Serum neutralization was also analyzed at Day 365 post-vaccination. Except for one subject in Group 5, all subjects had CHIKV-Luc SNA titers below the lower limit of detection prior to the Day 1 injection (FIG. 15 and Table 8). An immune response was observed in all groups by 7 days after the first active injection (i.e. Day 8 for Groups 1-4, Day 22 for Group 5-7, and Day 36 for Group 8). GMTs at these early time points ranged from 34.0 in Group 2 to 157.9 in Group 8. In Groups 1-4, the first peak GMT occurred at Day 22 (21 days after the first active injection). Groups 2, 3, and 4 had peak GMTs of 327.5, 1286.7, and 2123.4, demonstrating a dose-response effect. In addition, Group 4's peak GMT was higher than Group 1's peak GMT of 696.5 (p<0.0001), demonstrating the adjuvanting effect of alum. In Groups 5-7, the GMTs at Day 22 and Day 29 also demonstrated a dose-response. These GMT results are further summarized in FIG. 17. The single 40 ug dose (Group 8) had the best duration of response

TABLE 8
CHIKV-Luc SNA Geometric Mean Titers by Group and Time point

| Study | | | | | | | | | |
|---|---|---|---|---|---|---|---|---|---|
| Day | Statistic | Group 1 | Group 2 | Group 3 | Group 4 | Group 5 | Group 6 | Group 7 | Group 8 |
| Day 1 | N | 53 | 51 | 51 | 50 | 53 | 53 | 50 | 52 |
| | GMT | 7.5 | 7.5 | 7.5 | 7.5 | 7.8 | 7.5 | 7.5 | 7.5 |
| Day 8 | N | 53 | 50 | 49 | 48 | 52 | 52 | 50 | 51 |
| | GMT | 69.1 | 34.0 | 66.9 | 112.3 | 8.3 | 7.5 | 7.5 | 7.5 |
| Day 15 | N | 53 | 50 | 49 | 48 | 52 | 53 | 50 | 52 |
| | GMT | 391.3 | 179.2 | 513.4 | 895.9 | 7.7 | 7.5 | 7.6 | 7.5 |
| Day 22 | N | 51 | 48 | 49 | 47 | 52 | 53 | 50 | 52 |
| | GMT | 696.4 | 327.5 | 1262.6 | 2123.6 | 42.2 | 60.1 | 119.7 | 7.5 |
| Day 29 | N | 50 | 50 | 48 | 50 | 50 | 51 | 49 | 51 |
| | GMT | 403.8 | 272.8 | 837.6 | 1108.6 | 243.2 | 314.9 | 928.9 | 8.2 |
| Day 36 | N | 50 | 49 | 48 | 49 | 51 | 50 | 48 | 50 |
| | GMT | 2923.2 | 1579.7 | 2667.9 | 3264.0 | 1290.4 | 1966.4 | 3727.3 | 157.9 |
| Day 57 | N | 49 | 48 | 47 | 49 | 51 | 51 | 49 | 50 |
| | GMT | 1946.0 | 1084.8 | 1431.3 | 1884.4 | 914.0 | 1151.0 | 1613.2 | 1711.8 |
| Day 182 | N | 48 | 44 | 42 | 46 | 45 | 49 | 49 | 49 |
| | GMT | 340.5 | 196.1 | 371.3 | 454.2 | 254.3 | 303.4 | 408.1 | 462.5 |
| Day 365 | N | 44 | 42 | 39 | 45 | 42 | 46 | 45 | 47 |
| | GMT | 235.1 | 129.1 | 233.8 | 318.8 | 195.8 | 241.7 | 319.4 | 449.5 |
| Day 547 | N | 43 | — | — | 44 | — | — | — | 41 |
| | GMT | 162.4 | — | — | 239.6 | — | — | — | 271.6 |
| Day 760 | N | 42 | — | — | 41 | — | — | — | 39 |
| | GMT | 162.7 | — | — | 5513.4 | — | — | — | 254.2 |

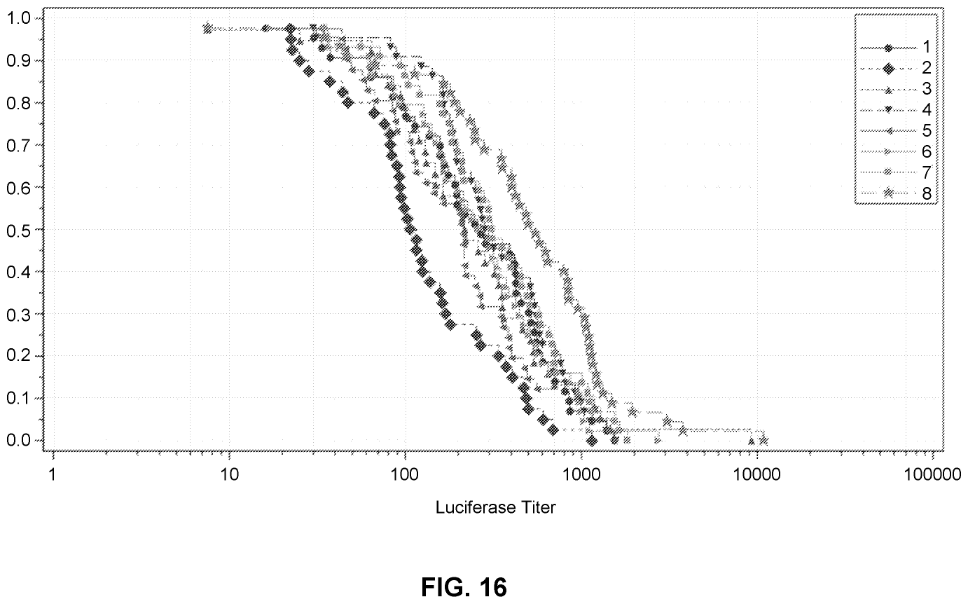

Following the second dose, the second peak GMT occurred at Day 36, again demonstrating a dose-response. Day 36 GMTs were also higher than all earlier GMTs in all 2-dose groups, demonstrating the strength of the anamnestic response to the second dose. At Day 57, which was 28 days after the last active injection for all groups, Group 1's GMT was 1946.0. The GMTs of the adjuvanted groups ranged from 914.0 in Group 5 to 1884.4 in Group 4, with Group 8 having the second highest GMT of 1711.8. GMTs declined in all groups from Day 57 to Day 182, at which point they ranged from 191.7 in Group 2 to 462.5 in Group 8. GMTs further declined at Day 365 in all groups except Group 8, which had a GMT of 466.2, higher than Group 1's GMT of 244.8 (p=0.0058). The reverse cumulative distribution of titers at day 365 for all groups are shown in FIG. 16. The highest titers were present in Group 8. These results show that Group 8 (single dose of 40 μg/adj.) sustained the highest titer over the 365 day period compared to Groups 1-7 and 9, which each were administered two separate doses.

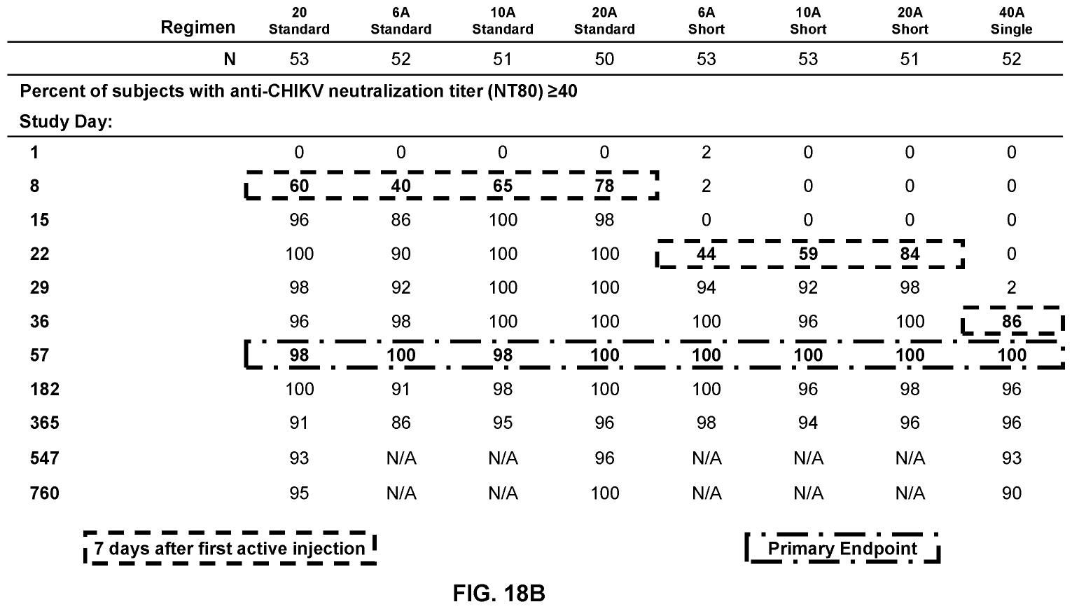

An analysis of seroresponse rate (SR) was performed, using a threshold NT80 of 40 or greater. This threshold was selected based on assay comparisons and epidemiology data. Seroresponse results are shown with patients having CHIK-Luc seropositivity≥40 (Table 9), ≥160 (Table 10), and ≥640 (Table 11). The calculated percentages of subjects in each Group with anti-CHIKV neutralization titer (NT80)≥15 and ≥40 is shown in FIG. 18A and FIG. 18B, respectively.

TABLE 9
Seroresponse Rates (NT80 ≥ 40) by Group and Timepoint

| Study | | | | | | | | | |
|---|---|---|---|---|---|---|---|---|---|
| Day | Statistic | Group 1 | Group 2 | Group 3 | Group 4 | Group 5 | Group 6 | Group 7 | Group 8 |
| Day 1 | N | 53 | 51 | 50 | 50 | 53 | 53 | 50 | 52 |
| | n (%) | 0 | 0 | 0 | 0 | 1 (1-9) | 0 | 0 | 0 |
| Day 8 | N | 53 | 50 | 49 | 49 | 52 | 52 | 50 | 51 |
| | n (%) | 32 (60.4) | 20 (40.0) | 32 (65.3) | 38 (77.6) | 1 (1-9) | 0 | 0 | 0 |
| Day 15 | N | 53 | 50 | 49 | 48 | 52 | 53 | 50 | 52 |
| | n (%) | 51 (96.2) | 43 (86.0) | 49 (100) | 47 (97.9) | 0 | 0 | 0 | 0 |
| Day 22 | N | 51 | 48 | 49 | 47 | 52 | 53 | 50 | 52 |
| | n (%) | 51 (100) | 43 (89.6) | 49 (100) | 47 (100) | 23 (44.2) | 31 (58.5) | 42 (84.0) | 0 |
| Day 29 | N | 50 | 50 | 48 | 50 | 50 | 51 | 49 | 51 |
| | n (%) | 49 (98.0) | 46 (92.0) | 48 (100) | 50 (100) | 47 (94.0) | 47 (92.2) | 48 (98.0) | 1 (2.0) |
| Day 36 | N | 50 | 49 | 48 | 49 | 51 | 50 | 48 | 50 |
| | n (%) | 48 (96.0) | 48 (98.0) | 48 (100) | 49 (100) | 51 (100) | 48 (96.0) | 48 (100) | 43 (86.0) |
| Day 57 | N | 49 | 48 | 47 | 49 | 51 | 51 | 49 | 50 |
| | n (%) | 48 (98.0) | 48 (100) | 46 (97.9) | 49 (100) | 51 (100) | 51 (100) | 49(100) | 50 (100) |
| Day 182 | N | 48 | 44 | 42 | 46 | 45 | 49 | 49 | 49 |
| | n (%) | 48 (100) | 40 (90.9) | 41 (97.6) | 46 (100) | 45(100) | 47 (95.9) | 48 (98.0) | 47 (95.9) |
| Day 365 | N | 44 | 42 | 39 | 45 | 42 | 46 | 45 | 47 |
| | n (%) | 40 (90.9) | 36 (85.7) | 37 (94.9) | 43 (95.6) | 41 (97.6) | 43 (93.5) | 43 (95.6) | 45 (95.7) |
| Day 547 | N | 43 | — | — | 44 | — | — | — | 41 |
| | n (%) | 40 (93.0) | | | 42 (95.5) | | | | 38 (92.7) |
| Day 760 | N | 42 | — | — | 41 | — | — | — | 39 |
| | n (%) | 40 (95.2) | | | 41 (100.0) | | | | 35 (89.7) |

TABLE 10
Percent of Subjects with CHIKV-Luc Seropositivity ≥160

| Study | Group | Group | Group | Group | Group | Group | Group | Group |
|---|---|---|---|---|---|---|---|---|
| Day | 1 | 2 | 3 | 4 | 5 | 6 | 7 | 8 |
| 1 | 0 | 0 | 0 | 0 | 0 | 0 | 0 | 0 |
| 8 | 34 | 12 | 25 | 39 | 2 | 0 | 0 | 0 |
| 15 | 77 | 46 | 76 | 92 | 0 | 0 | 0 | 0 |
| 22 | 84 | 75 | 96 | 100 | 15 | 17 | 40 | 0 |
| 29 | 78 | 72 | 96 | 96 | 58 | 82 | 94 | 2 |
| 36 | 96 | 94 | 98 | 100 | 100 | 96 | 98 | 44 |
| 57 | 98 | 94 | 98 | 100 | 98 | 98 | 98 | 100 |
| 182 | 75 | 61 | 86 | 89 | 64 | 78 | 86 | 82 |
| 365 | 66 | 36 | 59 | 87 | 60 | 67 | 80 | 87 |
| 547 | 54 | — | — | 73 | — | — | — | 76 |
| 760 | 50 | — | — | 100 | — | — | — | 72 |

TABLE 11
Percent of Subjects with CHIKV-Luc Seropositivity ≥640

| Study | Group | Group | Group | Group | Group | Group | Group | Group |
|---|---|---|---|---|---|---|---|---|
| Day | 1 | 2 | 3 | 4 | 5 | 6 | 7 | 8 |
| 1 | 0 | 0 | 0 | 0 | 0 | 0 | 0 | 0 |
| 8 | 9 | 4 | 4 | 6 | 0 | 0 | 0 | 0 |
| 15 | 30 | 24 | 43 | 63 | 0 | 0 | 0 | 0 |
| 22 | 53 | 31 | 71 | 89 | 6 | 4 | 10 | 0 |
| 29 | 38 | 26 | 60 | 74 | 22 | 29 | 65 | 0 |
| 36 | 92 | 84 | 96 | 98 | 73 | 88 | 96 | 18 |
| 57 | 90 | 73 | 92 | 92 | 61 | 82 | 88 | 88 |
| 182 | 23 | 11 | 26 | 33 | 13 | 25 | 29 | 45 |
| 365 | 18 | 7 | 18 | 22 | 12 | 17 | 27 | 43 |
| 547 | 5 | — | — | 5 | — | — | — | 22 |
| 760 | 7 | — | — | 98 | — | — | — | 21 |

At 7 days after the first active injection, the SR ranged from 40.0% in Group 2 to 86.0% in Group 8. The Group 4 SR of 77.6% was numerically higher than the Group 1 SR of 60.4%, consistent with the adjuvant effect shown by the GMT results, but missing statistical significance (p=0.0872). Group 1 and Group 4 reached an SR of 100% by Day 22. Group 8 did not have an analogous time point of 21 days after the first injection for comparison. Group 8 had the highest seroconversion at 7 days after one dose.

At Day 57, the SR was 100% in all groups except Group 1 (98.0%) and Group 3 (97.9%). By Day 182, the SR declined in some groups, ranging from 90.9% in Group 2 to 100% in Groups 1, 4 and 5. The SR in Group 8 was 95.9%. From Day 182 to Day 365, the SR declined in all groups, ranging from 85.0% in Group 2 to 97.6% in Group 5. The SR in Group 8 was 95.6%, slightly lower than at Day 182.

Additional analysis of SR was performed, using the more stringent threshold of 640. The SR 7 days after the first injection ranged from 3.8% in Group 6 to 18% in Group 8. At Day 57, the SR ranged from 60.8% in Group 5 to 91.8% in Group 4. The SR in Group 8 was 88.0%. By Day 182, the SR had declined in all groups, ranging from 11.4% in Group 2 to 44.9% in Group 8. By Day 365, the SR had again declined in all groups, but only minimally in Group 8, to 44.4%. Group 8 has the highest GMT at day 365 and the smallest decrease from Day 182.

Taken together, these data show that the immune response persisted to Day 365. Vaccinated groups had high (85%) Day 365 seropositivity at a threshold of 40.

### Example 7: Dosage Form for Human Clinical Study (Phase 3)

CHIKV VLPs were produced by transfecting HEK293 cells with the expression plasmid that encodes for the CHIKV structural protein genes as described in Example 1. In short, the expressed proteins self-assembled into VLPs that were then harvested and purified from the cell culture medium. The VLPs were purified by ultrafiltration, diafiltration, and anion exchange chromatography then sterile filtered. The VLPs were mixed with an appropriate amount of formulation buffer and then adsorbed on sterile aluminum hydroxide adjuvant (Alhydrogel® 2%).

CHIKV VLPs with aluminum hydroxide adjuvant was formulated as a sterile suspension for intramuscular administration available in 0.8 mL single dose pre-filled syringes. Each 0.8 mL dose contains approximately 40 μg of CHIKV-VLP, 300 μg of Aluminum (as aluminum hydroxide adjuvant), 59.7 mg of sucrose, 0.4 mg of potassium phosphate monobasic, 0.9 mg of potassium phosphate dibasic, 5.9 mg of sodium citrate dihydrate, and water for injection. The vaccine does not contain a preservative or antibiotics.

For this study, the vaccine will be administered intramuscularly as a single 0.8 mL dose. The vaccine should be administered intramuscularly in the deltoid region of the upper arm.

Effectiveness in subjects 12 through 64 years of age will be evaluated in a randomized, multicenter, placebo-controlled clinical study comparing the SNA responses following 1 dose of CHIKV-VLP+Alum vaccine or placebo. The study will be conducted in the U.S. and stratified by age (12 through 17 years of age, 18 through 45 years of age, and 46 through 64 years of age). This study will enroll about 3150 participants, who were randomized to receive a dose of CHIKV-VLP+Alum vaccine (n=2700) or placebo (n=450).

For all age groups, effectiveness will be inferred from the measurement of CHIKV NT80 serum neutralizing antibodies (SNA). The effectiveness of the vaccine in subjects will be assessed by comparing the SNA responses to immunization with CHIKV-VLP+Alum to those following immunization with placebo. The primary effectiveness endpoint can be SNA seroresponse 22 days after vaccination. In some aspects, seroresponse can be defined as a post-vaccination SNA titer≥40. Secondary endpoints included the SNA Geometric Mean Titer (GMT).

### Example 8: Human IgG from CHIK VLP-Vaccinated Subjects Protects Cynomolgus Macaques from Challenge

IgG was purified from pooled plasma collected plasma was collected between days 42-56 from human subjects (Group 9 from the Phase 2 clinical trial as described in Example 6) who had been vaccinated with 2 doses of 20 μg CHIK VLP with alum on Days 1 and 29. The purified IgG was administered to cynomolgus macaques at doses of 100, 15 and 5 mg/kg with 6 NHPs per dose group. A control group of 6 NHPs were administered a non-specific purified IgG. NHPs were challenged with CHIKV LR2006-OPY1 (10⁷ PFU) 24 hours after IgG administration. At time of challenge the average circulating SNA titers were 1:644, 1:101 and 1:38, in the 100, 15 and 5 mg/kg dose groups, respectively.

The results showed that purified IgG from CHK VLP vaccinated subjects protected NHPs from viremia as measured by plaque assay on serum from challenged animals as no infectious virus was observed at any time in these NHPs. All controls animals had detectable infectious virus. Joint pathology scores demonstrate that administration of IgG causes a reduction in joint pathology scores compared with control animals. RT-PCR analysis of joint tissue shows that administration of IgG reduces the amount of vRNA/g of tissue.

### Example 9: Human IgG from CHIK VLP-Vaccinated Subjects Tested in Cynomolgus Macaques Chikungunya Challenge

IgG will be purified from pooled plasma collected from human subjects who have been vaccinated with one dose of 40 μg CHIK VLP with alum. Purified IgG will be administered to cynomolgus macaques at various doses. A control group will be administered a non-specific purified IgG. NHPs will be challenged with CHIKV LR2006-OPY1 subcutaneously (e.g., 10⁵ PFU), 24 hours after IgG administration. NHPs will be evaluated for viremia, e.g., by plaque assay and/or qPCR of serum/plasma samples.

The Summary and Abstract sections may set forth one or more but not all exemplary embodiments of the present disclosure as contemplated by the inventor(s), and thus, are not intended to limit the present invention and the appended claims in any way.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined in accordance with the following claims and their equivalents.

What is claimed is:

1. A composition or vaccine comprising: (a) a virus-like particle (VLP) which comprises a capsid protein and an envelope protein derived from Chikungunya virus (CHIKV); (b) an aluminum hydroxide adjuvant; (c) a sugar; (d) a buffer and/or pH modifier; and (e) a carrier.

2. A composition or vaccine comprising a virus-like particle (VLP) which comprises a capsid protein and an envelope protein derived from Chikungunya virus (CHIKV), wherein the composition or vaccine (i) induces a neutralizing antibody response against CHIKV in a subject within 7 days after administration of a single dose of the composition or vaccine to the subject and/or (ii) induces a neutralizing antibody response against CHIKV which persists for at least 28 days, at least 6 months, at least 1 year, or at least 2 years after a single dose is administered to the subject.

3. The composition or vaccine of claim 2, further comprising an adjuvant.

4. The composition or vaccine of claim 3, wherein the adjuvant is an aluminum hydroxide adjuvant.

5. The composition or vaccine of any of claim 1, 3, or 4, wherein the adjuvant is aluminum hydroxide gel.

6. The composition or vaccine of any of the previous claims, wherein the capsid protein is a CHIKV C protein.

7. The composition or vaccine of any of the previous claims, wherein the envelope protein is a CHIKV protein selected from the group consisting of E3, E2, 6K, E1, and any combination thereof.

8. The composition or vaccine of any of the previous claims, wherein the VLP comprises CHIKV envelope proteins E2 and E1.

9. The composition or vaccine of any of the previous claims, wherein the VLP comprises CHIKV C, E2, and E1 proteins.

10. The composition or vaccine of claim 9, wherein the CHIKV C, E2, and E1 proteins are at least 95% of the VLP proteins in the composition or vaccine.

11. The composition or vaccine of any of the previous claims, wherein the VLP does not comprise CHIKV E3 and/or 6K.

12. The composition or vaccine of any of the previous claims, wherein the capsid and envelope proteins are derived CHIKV strain 37997.

13. The composition or vaccine of any of the previous claims which is a suspension suitable for intramuscular injection.

14. The composition or vaccine of any of the previous claims, wherein a/the single dose of the composition or vaccine comprises about 6 μg to 200 μg VLP.

15. The composition or vaccine of claim 14, wherein the single dose of the composition or vaccine comprises about 20 μg to 100 μg VLP.

16. The composition or vaccine of any of the previous claims, wherein a/the single dose of the composition or vaccine comprises about 25 μg/mL to 75 μg/mL VLP.

17. The composition or vaccine of any one of claims 14-16, wherein the single dose comprises about 40 μg or about 50 μg/mL VLP.

18. The composition or vaccine of any one of claims 5-17, wherein a/the single dose of the composition or vaccine comprises aluminum hydroxide gel in an amount of about 30 μg to 3000 μg.

19. The composition or vaccine of claim 18, wherein the single dose of the composition or vaccine comprises aluminum hydroxide gel in an amount of about 100 μg to 500 μg.

20. The composition or vaccine of claim 18 or 19, wherein the single dose comprises 250 μg/mL to 625 μg/mL aluminum hydroxide gel.

21. The composition or vaccine of any one of claims 18-20, wherein the single dose comprises about 300 μg or about 375 μg/mL aluminum hydroxide gel.

22. The composition or vaccine of any of the previous claims, wherein a/the single dose of the composition or vaccine comprises a volume of about 0.5 mL to 2 mL.

23. The composition or vaccine of claim 22, wherein the single dose of the composition or vaccine comprises a volume of about 0.5 mL to 1 mL.

24. The composition or vaccine of any of claims 2-23, further comprising a sugar.

25. The composition or vaccine of claim 24, wherein the sugar is selected from the group consisting of sucrose, lactose, and any combination thereof.

26. The composition or vaccine of claim 25, where the sugar is sucrose.

27. The composition or vaccine of any of claims 2-26, further comprising a buffer and/or pH modifier.

28. The composition or vaccine of claim 27, where the buffer and/or pH modifier is selected from the group consisting of potassium phosphate monobasic, potassium phosphate dibasic, sodium citrate dehydrate, and any combination thereof.

29. The composition or vaccine of any of the previous claims, wherein the composition or vaccine does not comprise a preservative or antibiotic.

30. The composition or vaccine of any of the previous claims, wherein a/the single dose of the composition or vaccine comprises: (a) about 6 μg to 60 μg of the VLP and (b) about 200 μg to 500 μg of a/the aluminum hydroxide, wherein the single dose volume is between about 0.5 mL to 1.0 mL.

31. The composition or vaccine of claim 30, wherein the single dose of the composition or vaccine comprises: (a) about 6 μg to 60 μg of the VLP; (b) about 200 μg to 500 μg of the aluminum hydroxide; (c) about 20 mg to 80 mg of sucrose; (d) about 0.2 mg to 1.0 mg of potassium phosphate monobasic; (e) about 0.2 mg to 1.5 mg of potassium phosphate diabasic; (f) about 2 mg to 10 mg sodium citrate dehydrate; and (g) water, wherein the single dose volume is between about 0.5 mL to 1.0 mL.

32. The composition or vaccine of any of the previous claims, wherein a/the single dose of the composition or vaccine is a volume of about 0.8 mL.

33. The composition or vaccine of any of the previous claims, wherein the VLP induces antibodies against homologous or heterologous strains of CHIKV.

34. The composition or vaccine of any of the previous claims, wherein the VLP induces antibodies against one or more CHIKV lineages selected from the group consisting of East/Central/South African (ECSA), West African and Asian.

35. The composition or vaccine of claims 1 and 5-34 which induces a neutralizing antibody response against CHIKV in a subject within 7 days after administration of a/the single dose of the composition or vaccine to the subject.

36. The composition or vaccine of any of claims 2-35, wherein the neutralizing antibody response against CHIKV is sustained for at least 28 days after a/the single dose is administered.

37. The composition or vaccine of claim 36, wherein the neutralizing antibody response against CHIKV is sustained for at least six months after the single dose is administered.

38. The composition or vaccine of claim 37, wherein the neutralizing antibody response against CHIKV is sustained for at least one year, at least 18 months, or at least 2 years after the single dose is administered.

39. The composition or vaccine of any of the previous claims, wherein the serum neutralizing antibody titer is ≥30 or ≥40 within 7 days after administration of a/the single dose of the composition or vaccine.

40. The composition or vaccine of any of the previous claims, wherein the composition or vaccine induces a neutralizing antibody response against CHIKV in a subject within 7 days after administration of the single dose of the composition.

41. The composition or vaccine of any of the previous claims, wherein the composition or vaccine induces a neutralizing antibody response against CHIKV which persists for at least 28 days, at least 6 months, at least 1 year, or at least 2 years after a single dose is administered to the subject.

42. The composition or vaccine of any of the previous claims, wherein the composition or vaccine (i) induces a neutralizing antibody response against CHIKV in a subject within 7 days after administration of a single dose of the composition or vaccine to the subject and (ii) induces a neutralizing antibody response against CHIKV which persists for at least 28 days, at least 6 months, at least 1 year, or at least 2 years after a single dose is administered to the subject.

43. A pre-filled syringe comprising a/the single dose of the composition or vaccine of any one of claims 1-42.

44. A method of inducing an immune response and/or protective immunity against Chikungunya virus (CHIKV) in a subject, comprising administering to the subject the composition or vaccine of any one of claims 1-42.

45. A method of inducing a neutralizing antibody response to Chikungunya virus (CHIKV) in a subject, comprising administering to the subject the composition or vaccine of any one of claims 1-42.

46. A method for treating, preventing, or reducing the risk of a Chikungunya infection in a subject, comprising administering to the subject an effective amount of the composition or vaccine of any one of claims 1-42.

47. A method of vaccinating a subject against a CHIKV infection, comprising administering to the subject an effective amount of the composition or vaccine of any one of claims 1-42.

48. A method of eliciting an immune response and/or protective immunity against Chikungunya virus (CHIKV) in a subject comprising: administering to the subject a composition or vaccine comprising (a) virus-like particles (VLPs) which comprise a capsid protein and an envelope protein derived from CHIKV and (b) an adjuvant, wherein a single dose of the composition or vaccine comprises about 6 μg to 200 μg VLPs, and wherein the administration of the single dose of the composition or vaccine (i) elicits production of CHIKV-specific neutralizing antibodies in the subject within 7 days of administration and/or (ii) induces a neutralizing antibody response against CHIKV which persists for at least 28 days, at least 6 months, at least 1 year, or at least 2 years after a single dose is administered to the subject.

49. A method of inducing protective immunity against Chikungunya virus (CHIKV) in a population comprising: administering to the population a composition or vaccine comprising (a) virus-like particles (VLPs) which comprise a capsid protein and an envelope protein derived from CHIKV and (b) an adjuvant, wherein a single dose of the composition or vaccine comprises about 6 μg to 200 μg VLPs, and wherein at least 60% of the population (i) produces a neutralizing antibody response against CHIKV within 7 days after administration of the single dose of the composition or vaccine and/or (ii) produces neutralizing antibodies against CHIKV which persists for at least 28 days, at least 6 months, at least 1 year, or at least 2 years after a single dose is administered to the subject.

50. The method of claim 49, wherein at least 50%, at least 60%, at least 70%, at least 80%, at least 90%, at least 95%, at least 96%, at least 97%, or at least 98% of the population develops a serum neutralizing antibody titer≥30 or ≥40 within 7 days after administration of a single dose of the composition or vaccine.

51. The method of claim 49 or 50, wherein at least 40%, at least 50%, or at least 60% of the population maintains a serum neutralizing antibody titer≥30 or ≥40 for at least 28 days, at least 6 months, at least one year, or at least two years after administration of a single dose of the composition or vaccine.

52. The method of any one of claims 44-51, wherein a/the single dose comprises 20 μg to 200 μg VLP.

53. The method of any of claims 44-52, wherein a/the single dose comprises about 25 μg/mL to 75 μg/mL VLP.

54. The method of claim 53, wherein the single dose comprises about 40 μg or about 50 μg/mL VLP.

55. The method of any one of claims 44-54, wherein the composition or vaccine comprises 30 μg-3000 μg aluminum hydroxide gel

56. The method of claim 55, wherein the aluminum hydroxide gel is in an amount of about 250 μg/mL to 625 μg/mL.

57. The method of claim any one of claims 44-56, wherein the composition or vaccine comprises about 300 μg or 375 μg/mL aluminum hydroxide gel.

58. The method of any one of claims 44-57, wherein the composition or vaccine comprises: (a) about 6 to 40 μg of VLP; (b) about 200 to 500 μg of aluminum hydroxide; (c) a sugar; (d) a buffer and/or a pH modifier; and (e) and water.

59. The method of claim 58, wherein the composition or vaccine comprises: (a) about 6 to 40 μg of VLP; (b) about 200 to 500 μg of aluminum hydroxide; (c) about 20 to 80 mg of sucrose; (d) about 0.2 to 1.0 mg of potassium phosphate monobasic; (e) about 0.2 to 1.5 mg of potassium phosphate diabasic; (f) about 2 to 10 mg sodium citrate dehydrate; and (g) and water.

60. The method of any one of claims 44-59, wherein a/the first dose is administered in an amount of about 0.5 mL to 1 mL.

61. The method of claim 60, wherein the first does is administered in an amount of about 0.8 mL.

62. The method of any one of claims 44-61, wherein the neutralizing antibody response vaccinates the subject against multiple serotypes of CHIKV.

63. The method of any one of claims 44-62, herein the neutralizing antibody response against CHIKV is sustained for at least 28 days, at least 6 months, at least 1 year, or at least 2 years after the single dose is administered.

64. The method of any one of claims 44-63, wherein a second dose of the composition or vaccine is administered to the subject at least 28 days, at least 6 months, or at least 1 year after the single dose.

65. The method of claim 64, wherein the neutralizing antibody response against CHIKV is sustained for at least six months after administration of the second dose.

66. The method of claim 65, wherein the neutralizing antibody response against CHIKV is sustained for at least one year after administration of the second dose.

67. The method of any one of claims 44-66, wherein at least 60%, at least 70%, at least 80%, at least 90%, at least 95%, or at least 98% of the subjects produced a neutralizing antibody response to CHIKV 7 days after administration of the composition or vaccine.

68. The method of any one of claims 44-67, wherein the seroresponse rate is at least 60%, at least 70%, at least 80%, at least 90%, or at least 95% 7 days, 28 days, one year, or two years after administration of the composition or vaccine.

69. The method of any of one of claims 44-68, wherein the serum neutralizing antibody titer is ≥30 or ≥40.

70. The method of any one of claims 44-69, wherein the composition or vaccine is administered intramuscularly.

71. The method of any one of claims 44-70, wherein the composition or vaccine is administered in an effective amount.

72. The method of any one of claims 44-71, wherein the method elicits production of CHIKV-specific neutralizing antibodies in the subject within 7 days of administration and induces the neutralizing antibody response against CHIKV which persists for at least 28 days, at least 6 months, at least 1 year, or at least 2 years after a single dose is administered to the subject.

73. The composition, vaccine or method of any of the previous claims, wherein a/the subject is a human.

74. The composition, vaccine or method of any of the previous claims, wherein a/the subject had no exposure to a CHIKV antigen or a CHIKV vaccine prior to administration of the composition or vaccine.

75. The composition, vaccine or method of any of the previous claims, wherein a/the subject has no detectable CHIKV antibody titer prior to administration of the composition or vaccine.

76. The composition, vaccine or method of any of the previous claims, wherein a/the subject has a detectable CHIKV antibody titer≤5 prior to administration of the composition or vaccine.
